SEQUENCE LISTING (199 sequences)

SEQ ID NO: 1 - protein, 32 aa, synthetic construct
  REGION 1..32: note = Synthetic: VelA
VAQLEEKVKTLRAENYELKSEVQRLEEQVAQL

SEQ ID NO: 2 - protein, 32 aa, synthetic construct
  REGION 1..32: note = Synthetic: VelB
VDELQAEVDQLEDENYALKTKVAQLRKKVEKL

SEQ ID NO: 3 - protein, 38 aa, synthetic construct
  REGION 1..38: note = Synthetic: VelA with amino-terminal QGASTT
QGASTTVAQLEEKVKTLRAENYELKSEVQRLEEQVAQL

SEQ ID NO: 4 - protein, 38 aa, synthetic construct
  REGION 1..38: note = Synthetic: VelB with amino-terminal QGASTS
QGASTSVDELQAEVDQLEDENYALKTKVAQLRKKVEKL

SEQ ID NO: 5 - protein, 32 aa, synthetic construct
  REGION 1..32: note = Synthetic: VelA V7N
NAQLEEKVKTLRAENYELKSEVQRLEEQVAQL

SEQ ID NO: 6 - protein, 32 aa, synthetic construct
  REGION 1..32: note = Synthetic: VelA V14I
VAQLEEKIKTLRAENYELKSEVQRLEEQVAQL

SEQ ID NO: 7 - protein, 32 aa, synthetic construct
  REGION 1..32: note = Synthetic: VelA V14A
VAQLEEKAKTLRAENYELKSEVQRLEEQVAQL

SEQ ID NO: 8 - protein, 32 aa, synthetic construct
  REGION 1..32: note = Synthetic: VelA L17A
VAQLEEKVKTARAENYELKSEVQRLEEQVAQL

SEQ ID NO: 9 - protein, 32 aa, synthetic construct
  REGION 1..32: note = Synthetic: VelA Y22C
VAQLEEKVKTLRAENCELKSEVQRLEEQVAQL

SEQ ID NO: 10 - protein, 32 aa, synthetic construct
  REGION 1..32: note = Synthetic: VelA Y22S
VAQLEEKVKTLRAENSELKSEVQRLEEQVAQL

SEQ ID NO: 11 - protein, 32 aa, synthetic construct
  REGION 1..32: note = Synthetic: VelA L24A
VAQLEEKVKTLRAENYEAKSEVQRLEEQVAQL

SEQ ID NO: 12 - protein, 32 aa, synthetic construct
  REGION 1..32: note = Synthetic: VelA V28I
VAQLEEKVKTLRAENYELKSEIQRLEEQVAQL

SEQ ID NO: 13 - protein, 32 aa, synthetic construct
  REGION 1..32: note = Synthetic: VelA V28A
VAQLEEKVKTLRAENYELKSEAQRLEEQVAQL

SEQ ID NO: 14 - protein, 32 aa, synthetic construct
  REGION 1..32: note = Synthetic: VelA V28N
VAQLEEKVKTLRAENYELKSENQRLEEQVAQL

SEQ ID NO: 15 - protein, 32 aa, synthetic construct
  REGION 1..32: note = Synthetic: VelA L31A
VAQLEEKVKTLRAENYELKSEVQRAEEQVAQL

SEQ ID NO: 16 - protein, 32 aa, synthetic construct
  REGION 1..32: note = Synthetic: VelA V35I
VAQLEEKVKTLRAENYELKSEVQRLEEQIAQL

SEQ ID NO: 17 - protein, 32 aa, synthetic construct
  REGION 1..32: note = Synthetic: VelA V35A
VAQLEEKVKTLRAENYELKSEVQRLEEQAAQL

SEQ ID NO: 18 - protein, 32 aa, synthetic construct
  REGION 1..32: note = Synthetic: VelA V35N
VAQLEEKVKTLRAENYELKSEVQRLEEQNAQL

SEQ ID NO: 19 - protein, 32 aa, synthetic construct
  REGION 1..32: note = Synthetic: VelA E11Q
VAQLQEKVKTLRAENYELKSEVQRLEEQVAQL

SEQ ID NO: 20 - protein, 32 aa, synthetic construct
  REGION 1..32: note = Synthetic: VelA E11N
VAQLNEKVKTLRAENYELKSEVQRLEEQVAQL

SEQ ID NO: 21 - protein, 32 aa, synthetic construct
  REGION 1..32: note = Synthetic: VelA E11K
VAQLKEKVKTLRAENYELKSEVQRLEEQVAQL

SEQ ID NO: 22 - protein, 32 aa, synthetic construct
  REGION 1..32: note = Synthetic: VelA E12Q
VAQLEQKVKTLRAENYELKSEVQRLEEQVAQL

SEQ ID NO: 23 - protein, 32 aa, synthetic construct
  REGION 1..32: note = Synthetic: VelA E12N
VAQLENKVKTLRAENYELKSEVQRLEEQVAQL

SEQ ID NO: 24 - protein, 32 aa, synthetic construct
  REGION 1..32: note = Synthetic: VelA E12K
VAQLEKKVKTLRAENYELKSEVQRLEEQVAQL

SEQ ID NO: 25 - protein, 32 aa, synthetic construct
  REGION 1..32: note = Synthetic: VelA T16A
VAQLEEKVKALRAENYELKSEVQRLEEQVAQL

SEQ ID NO: 26 - protein, 32 aa, synthetic construct
  REGION 1..32: note = Synthetic: VelA E20Q
VAQLEEKVKTLRAQNYELKSEVQRLEEQVAQL

SEQ ID NO: 27 - protein, 32 aa, synthetic construct
  REGION 1..32: note = Synthetic: VelAE20N
VAQLEEKVKTLRANNYELKSEVQRLEEQVAQL

SEQ ID NO: 28 - protein, 32 aa, synthetic construct
  REGION 1..32: note = Synthetic: VelA E20K
VAQLEEKVKTLRAKNYELKSEVQRLEEQVAQL

SEQ ID NO: 29 - protein, 32 aa, synthetic construct
  REGION 1..32: note = Synthetic: VelA E23Q
VAQLEEKVKTLRAENYQLKSEVQRLEEQVAQL

SEQ ID NO: 30 - protein, 32 aa, synthetic construct
  REGION 1..32: note = Synthetic: VelA E32Q
VAQLEEKVKTLRAENYELKSEVQRLQEQVAQL

SEQ ID NO: 31 - protein, 32 aa, synthetic construct
  REGION 1..32: note = Synthetic: VelA E32N
VAQLEEKVKTLRAENYELKSEVQRLNEQVAQL

SEQ ID NO: 32 - protein, 32 aa, synthetic construct
  REGION 1..32: note = Synthetic: VelA E32K
VAQLEEKVKTLRAENYELKSEVQRLKEQVAQL

SEQ ID NO: 33 - protein, 32 aa, synthetic construct
  REGION 1..32: note = Synthetic: VelA E33Q
VAQLEEKVKTLRAENYELKSEVQRLEQQVAQL

SEQ ID NO: 34 - protein, 32 aa, synthetic construct
  REGION 1..32: note = Synthetic: VelA E33N
VAQLEEKVKTLRAENYELKSEVQRLENQVAQL

SEQ ID NO: 35 - protein, 32 aa, synthetic construct
  REGION 1..32: note = Synthetic: VelA E33K
VAQLEEKVKTLRAENYELKSEVQRLEKQVAQL

SEQ ID NO: 36 - protein, 32 aa, synthetic construct
  REGION 1..32: note = Synthetic: VelA A8H
VHQLEEKVKTLRAENYELKSEVQRLEEQVAQL

SEQ ID NO: 37 - protein, 32 aa, synthetic construct
  REGION 1..32: note = Synthetic: VelA A8K
VKQLEEKVKTLRAENYELKSEVQRLEEQVAQL

SEQ ID NO: 38 - protein, 32 aa, synthetic construct
  REGION 1..32: note = Synthetic: VelA Q9H
VAHLEEKVKTLRAENYELKSEVQRLEEQVAQL

SEQ ID NO: 39 - protein, 32 aa, synthetic construct
  REGION 1..32: note = Synthetic: VelA Q9K
VAKLEEKVKTLRAENYELKSEVQRLEEQVAQL

SEQ ID NO: 40 - protein, 32 aa, synthetic construct
  REGION 1..32: note = Synthetic: VelA E11H
VAQLHEKVKTLRAENYELKSEVQRLEEQVAQL

SEQ ID NO: 41 - protein, 32 aa, synthetic construct
  REGION 1..32: note = Synthetic: VelA E12H
VAQLEHKVKTLRAENYELKSEVQRLEEQVAQL

SEQ ID NO: 42 - protein, 32 aa, synthetic construct
  REGION 1..32: note = Synthetic: VelA K15H
VAQLEEKVHTLRAENYELKSEVQRLEEQVAQL

SEQ ID NO: 43 - protein, 32 aa, synthetic construct
  REGION 1..32: note = Synthetic: VelA K15D
VAQLEEKVDTLRAENYELKSEVQRLEEQVAQL

SEQ ID NO: 44 - protein, 32 aa, synthetic construct
  REGION 1..32: note = Synthetic: VelA A19H
VAQLEEKVKTLRHENYELKSEVQRLEEQVAQL

SEQ ID NO: 45 - protein, 32 aa, synthetic construct
  REGION 1..32: note = Synthetic: VelA K25H
VAQLEEKVKTLRAENYELHSEVQRLEEQVAQL

SEQ ID NO: 46 - protein, 32 aa, synthetic construct
  REGION 1..32: note = Synthetic: VelA E33H
VAQLEEKVKTLRAENYELKSEVQRLEHQVAQL

SEQ ID NO: 47 - protein, 32 aa, synthetic construct
  REGION 1..32: note = Synthetic: VelA A36H
VAQLEEKVKTLRAENYELKSEVQRLEEQVHQL

SEQ ID NO: 48 - protein, 32 aa, synthetic construct
  REGION 1..32: note = Synthetic: VelA K25R
VAQLEEKVKTLRAENYELRSEVQRLEEQVAQL

SEQ ID NO: 49 - protein, 32 aa, synthetic construct
  REGION 1..32: note = Synthetic: VelAL24A/L31A
VAQLEEKVKTLRAENYEAKSEVQRAEEQVAQL

SEQ ID NO: 50 - protein, 32 aa, synthetic construct
  REGION 1..32: note = Synthetic: VelAL17A/L31A
VAQLEEKVKTARAENYELKSEVQRAEEQVAQL

SEQ ID NO: 51 - protein, 32 aa, synthetic construct
  REGION 1..32: note = Synthetic: VelAL17A/L24A/L31A
VAQLEEKVKTARAENYEAKSEVQRAEEQVAQL

SEQ ID NO: 52 - protein, 32 aa, synthetic construct
  REGION 1..32: note = Synthetic: VelAL31A/V35A
VAQLEEKVKTLRAENYELKSEVQRAEEQAAQL

SEQ ID NO: 53 - protein, 32 aa, synthetic construct
  REGION 1..32: note = Synthetic: VelAV28A/L31A
VAQLEEKVKTLRAENYELKSEAQRAEEQVAQL

SEQ ID NO: 54 - protein, 32 aa, synthetic construct
  REGION 1..32: note = Synthetic: VelAV28A/L31A/V35A
VAQLEEKVKTLRAENYELKSEAQRAEEQAAQL

SEQ ID NO: 55 - protein, 32 aa, synthetic construct
  REGION 1..32: note = Synthetic: VelAL24A/V28A/L31A
VAQLEEKVKTLRAENYEAKSEAQRAEEQVAQL

SEQ ID NO: 56 - protein, 32 aa, synthetic construct
  REGION 1..32: note = Synthetic: VelAL31A/E33K
VAQLEEKVKTLRAENYELKSEVQRAEKQVAQL

SEQ ID NO: 57 - protein, 32 aa, synthetic construct
  REGION 1..32: note = Synthetic: VelAL24I
VAQLEEKVKTLRAENYEIKSEVQRLEEQVAQL

SEQ ID NO: 58 - protein, 32 aa, synthetic construct
  REGION 1..32: note = Synthetic: VelAL31I
VAQLEEKVKTLRAENYELKSEVQRIEEQVAQL

SEQ ID NO: 59 - protein, 32 aa, synthetic construct
  REGION 1..32: note = Synthetic: VelB V7N
NDELQAEVDQLEDENYALKTKVAQLRKKVEKL

SEQ ID NO: 60 - protein, 32 aa, synthetic construct
  REGION 1..32: note = Synthetic: VelB V14A
VDELQAEADQLEDENYALKTKVAQLRKKVEKL

SEQ ID NO: 61 - protein, 32 aa, synthetic construct
  REGION 1..32: note = Synthetic: VelB V14I
VDELQAEIDQLEDENYALKTKVAQLRKKVEKL

SEQ ID NO: 62 - protein, 32 aa, synthetic construct
  REGION 1..32: note = Synthetic: VelB V14N
VDELQAENDQLEDENYALKTKVAQLRKKVEKL

SEQ ID NO: 63 - protein, 32 aa, synthetic construct
  REGION 1..32: note = Synthetic: VelB L17I
VDELQAEVDQIEDENYALKTKVAQLRKKVEKL

SEQ ID NO: 64 - protein, 32 aa, synthetic construct
  REGION 1..32: note = Synthetic: VelB Y22C
VDELQAEVDQLEDENCALKTKVAQLRKKVEKL

SEQ ID NO: 65 - protein, 32 aa, synthetic construct
  REGION 1..32: note = Synthetic: VelB Y22S
VDELQAEVDQLEDENSALKTKVAQLRKKVEKL

SEQ ID NO: 66 - protein, 32 aa, synthetic construct
  REGION 1..32: note = Synthetic: VelB L24I
VDELQAEVDQLEDENYAIKTKVAQLRKKVEKL

SEQ ID NO: 67 - protein, 32 aa, synthetic construct
  REGION 1..32: note = Synthetic: VelB V28A
VDELQAEVDQLEDENYALKTKAAQLRKKVEKL

SEQ ID NO: 68 - protein, 32 aa, synthetic construct
  REGION 1..32: note = Synthetic: VelB V28I
VDELQAEVDQLEDENYALKTKIAQLRKKVEKL

SEQ ID NO: 69 - protein, 32 aa, synthetic construct
  REGION 1..32: note = Synthetic: VelB V28N
VDELQAEVDQLEDENYALKTKNAQLRKKVEKL

SEQ ID NO: 70 - protein, 32 aa, synthetic construct
  REGION 1..32: note = Synthetic: VelB L31I
VDELQAEVDQLEDENYALKTKVAQIRKKVEKL

SEQ ID NO: 71 - protein, 32 aa, synthetic construct
  REGION 1..32: note = Synthetic: VelB V35A
VDELQAEVDQLEDENYALKTKVAQLRKKAEKL

SEQ ID NO: 72 - protein, 32 aa, synthetic construct
  REGION 1..32: note = Synthetic: VelB V35I
VDELQAEVDQLEDENYALKTKVAQLRKKIEKL

SEQ ID NO: 73 - protein, 32 aa, synthetic construct
  REGION 1..32: note = Synthetic: VelBV35N
VDELQAEVDQLEDENYALKTKVAQLRKKNEKL

SEQ ID NO: 74 - protein, 32 aa, synthetic construct
  REGION 1..32: note = Synthetic: VelB D8Q
VQELQAEVDQLEDENYALKTKVAQLRKKVEKL

SEQ ID NO: 75 - protein, 32 aa, synthetic construct
  REGION 1..32: note = Synthetic: VelB D8N
VNELQAEVDQLEDENYALKTKVAQLRKKVEKL

SEQ ID NO: 76 - protein, 32 aa, synthetic construct
  REGION 1..32: note = Synthetic: VelB E9N
VDNLQAEVDQLEDENYALKTKVAQLRKKVEKL

SEQ ID NO: 77 - protein, 32 aa, synthetic construct
  REGION 1..32: note = Synthetic: VelB E9Q
VDQLQAEVDQLEDENYALKTKVAQLRKKVEKL

SEQ ID NO: 78 - protein, 32 aa, synthetic construct
  REGION 1..32: note = Synthetic: VelB E13R
VDELQARVDQLEDENYALKTKVAQLRKKVEKL

SEQ ID NO: 79 - protein, 32 aa, synthetic construct
  REGION 1..32: note = Synthetic: VelB E13N
VDELQANVDQLEDENYALKTKVAQLRKKVEKL

SEQ ID NO: 80 - protein, 32 aa, synthetic construct
  REGION 1..32: note = Synthetic: VelB E13Q
VDELQAQVDQLEDENYALKTKVAQLRKKVEKL

SEQ ID NO: 81 - protein, 32 aa, synthetic construct
  REGION 1..32: note = Synthetic: VelB D15E
VDELQAEVEQLEDENYALKTKVAQLRKKVEKL

SEQ ID NO: 82 - protein, 32 aa, synthetic construct
  REGION 1..32: note = Synthetic: VelB D15S
VDELQAEVSQLEDENYALKTKVAQLRKKVEKL

SEQ ID NO: 83 - protein, 32 aa, synthetic construct
  REGION 1..32: note = Synthetic: VelB D15N
VDELQAEVNQLEDENYALKTKVAQLRKKVEKL

SEQ ID NO: 84 - protein, 32 aa, synthetic construct
  REGION 1..32: note = Synthetic: VelB D19S
VDELQAEVDQLESENYALKTKVAQLRKKVEKL

SEQ ID NO: 85 - protein, 32 aa, synthetic construct
  REGION 1..32: note = Synthetic: VelB D19N
VDELQAEVDQLENENYALKTKVAQLRKKVEKL

SEQ ID NO: 86 - protein, 32 aa, synthetic construct
  REGION 1..32: note = Synthetic: VelB E20K
VDELQAEVDQLEDKNYALKTKVAQLRKKVEKL

SEQ ID NO: 87 - protein, 32 aa, synthetic construct
  REGION 1..32: note = Synthetic: VelB E20N
VDELQAEVDQLEDNNYALKTKVAQLRKKVEKL

SEQ ID NO: 88 - protein, 32 aa, synthetic construct
  REGION 1..32: note = Synthetic: VelB E20Q
VDELQAEVDQLEDQNYALKTKVAQLRKKVEKL

SEQ ID NO: 89 - protein, 32 aa, synthetic construct
  REGION 1..32: note = Synthetic: VelB R32K
VDELQAEVDQLEDENYALKTKVAQLKKKVEKL

SEQ ID NO: 90 - protein, 32 aa, synthetic construct
  REGION 1..32: note = Synthetic: VelB K33Q
VDELQAEVDQLEDENYALKTKVAQLRQKVEKL

SEQ ID NO: 91 - protein, 32 aa, synthetic construct
  REGION 1..32: note = Synthetic: VelB K33E
VDELQAEVDQLEDENYALKTKVAQLREKVEKL

SEQ ID NO: 92 - protein, 32 aa, synthetic construct
  REGION 1..32: note = Synthetic: VelB K37Q
VDELQAEVDQLEDENYALKTKVAQLRKKVEQL

SEQ ID NO: 93 - protein, 32 aa, synthetic construct
  REGION 1..32: note = Synthetic: VelB K37E
VDELQAEVDQLEDENYALKTKVAQLRKKVEEL

SEQ ID NO: 94 - protein, 32 aa, synthetic construct
  REGION 1..32: note = Synthetic: VelB D8K
VKELQAEVDQLEDENYALKTKVAQLRKKVEKL

SEQ ID NO: 95 - protein, 32 aa, synthetic construct
  REGION 1..32: note = Synthetic: VelB D8H
VHELQAEVDQLEDENYALKTKVAQLRKKVEKL

SEQ ID NO: 96 - protein, 32 aa, synthetic construct
  REGION 1..32: note = Synthetic: VelB E9K
VDKLQAEVDQLEDENYALKTKVAQLRKKVEKL

SEQ ID NO: 97 - protein, 32 aa, synthetic construct
  REGION 1..32: note = Synthetic: VelB E9H
VDHLQAEVDQLEDENYALKTKVAQLRKKVEKL

SEQ ID NO: 98 - protein, 32 aa, synthetic construct
  REGION 1..32: note = Synthetic: VelB Q11K
VDELKAEVDQLEDENYALKTKVAQLRKKVEKL

SEQ ID NO: 99 - protein, 32 aa, synthetic construct
  REGION 1..32: note = Synthetic: VelB Q11H
VDELHAEVDQLEDENYALKTKVAQLRKKVEKL

SEQ ID NO: 100 - protein, 32 aa, synthetic construct
  REGION 1..32: note = Synthetic: VelB A12K
VDELQKEVDQLEDENYALKTKVAQLRKKVEKL

SEQ ID NO: 101 - protein, 32 aa, synthetic construct
  REGION 1..32: note = Synthetic: VelB A12H
VDELQHEVDQLEDENYALKTKVAQLRKKVEKL

SEQ ID NO: 102 - protein, 32 aa, synthetic construct
  REGION 1..32: note = Synthetic: VelB D15K
VDELQAEVKQLEDENYALKTKVAQLRKKVEKL

SEQ ID NO: 103 - protein, 32 aa, synthetic construct
  REGION 1..32: note = Synthetic: VelB D15H
VDELQAEVHQLEDENYALKTKVAQLRKKVEKL

SEQ ID NO: 104 - protein, 32 aa, synthetic construct
  REGION 1..32: note = Synthetic: VelB D19K
VDELQAEVDQLEKENYALKTKVAQLRKKVEKL

SEQ ID NO: 105 - protein, 32 aa, synthetic construct
  REGION 1..32: note = Synthetic: VelB D19H
VDELQAEVDQLEHENYALKTKVAQLRKKVEKL

SEQ ID NO: 106 - protein, 32 aa, synthetic construct
  REGION 1..32: note = Synthetic: VelB K25H
VDELQAEVDQLEDENYALHTKVAQLRKKVEKL

SEQ ID NO: 107 - protein, 32 aa, synthetic construct
  REGION 1..32: note = Synthetic: VelB K34H
VDELQAEVDQLEDENYALKTKVAQLRKHVEKL

SEQ ID NO: 108 - protein, 32 aa, synthetic construct
  REGION 1..32: note = Synthetic: VelB E36H
VDELQAEVDQLEDENYALKTKVAQLRKKVHKL

SEQ ID NO: 109 - protein, 32 aa, synthetic construct
  REGION 1..32: note = Synthetic: VelB K25R
VDELQAEVDQLEDENYALRTKVAQLRKKVEKL

SEQ ID NO: 110 - protein, 32 aa, synthetic construct
  REGION 1..32: note = Synthetic: VelBL24I/L31I
VDELQAEVDQLEDENYAIKTKVAQIRKKVEKL

SEQ ID NO: 111 - protein, 32 aa, synthetic construct
  REGION 1..32: note = Synthetic: VelBL17I/L31I
VDELQAEVDQIEDENYALKTKVAQIRKKVEKL

SEQ ID NO: 112 - protein, 32 aa, synthetic construct
  REGION 1..32: note = Synthetic: VelBL17I/L24I/L31I
VDELQAEVDQIEDENYAIKTKVAQIRKKVEKL

SEQ ID NO: 113 - protein, 32 aa, synthetic construct
  REGION 1..32: note = Synthetic: VelBL31I/V35I
VDELQAEVDQLEDENYALKTKVAQIRKKIEKL

SEQ ID NO: 114 - protein, 32 aa, synthetic construct
  REGION 1..32: note = Synthetic: VelBV28I/L31I
VDELQAEVDQLEDENYALKTKIAQIRKKVEKL

SEQ ID NO: 115 - protein, 32 aa, synthetic construct
  REGION 1..32: note = Synthetic: VelBV28I/L31I/V35I
VDELQAEVDQLEDENYALKTKIAQIRKKIEKL

SEQ ID NO: 116 - protein, 32 aa, synthetic construct
  REGION 1..32: note = Synthetic: VelBD8K/E36H
VKELQAEVDQLEDENYALKTKVAQLRKKVHKL

SEQ ID NO: 117 - protein, 32 aa, synthetic construct
  REGION 1..32: note = Synthetic: VelBD8K/E36H/L31I
VKELQAEVDQLEDENYALKTKVAQIRKKVHKL

SEQ ID NO: 118 - protein, 32 aa, synthetic construct
  REGION 1..32: note = Synthetic: VelBD8K/D15K/L31I
VKELQAEVKQLEDENYALKTKVAQIRKKVEKL

SEQ ID NO: 119 - protein, 32 aa, synthetic construct
  REGION 1..32: note = Synthetic: VelBD15K/E36H/L31I
VDELQAEVKQLEDENYALKTKVAQIRKKVHKL

SEQ ID NO: 120 - protein, 32 aa, synthetic construct
  REGION 1..32: note = Synthetic: VelBL24A
VDELQAEVDQLEDENYAAKTKVAQLRKKVEKL

SEQ ID NO: 121 - protein, 32 aa, synthetic construct
  REGION 1..32: note = Synthetic: VelBL31A
VDELQAEVDQLEDENYALKTKVAQARKKVEKL

SEQ ID NO: 122 - protein, 8 aa, synthetic construct
  REGION 1..8: note = Synthetic: IPV cleavage site
IPVSLRSG

SEQ ID NO: 123 - protein, 6 aa, synthetic construct
  REGION 1..6: note = Synthetic: M2 cleavage site
GPLGVR

SEQ ID NO: 124 - protein, 8 aa, synthetic construct
  REGION 1..8: note = Synthetic: MMP cleavage site 1
LSGRSDNY

SEQ ID NO: 125 - protein, 5 aa, synthetic construct
  REGION 1..5: note = Synthetic: MMP cleavage site 2
PLGVR

SEQ ID NO: 126 - protein, 5 aa, synthetic construct
  REGION 1..5: note = Synthetic: Linker 1
GSGGS

SEQ ID NO: 127 - protein, 4 aa, synthetic construct
  REGION 1..4: note = Synthetic: Linker 2
GGGS

SEQ ID NO: 128 - protein, 12 aa, synthetic construct
  REGION 1..12: note = Synthetic: Linker 3
SGGGGGGGGGGS

SEQ ID NO: 129 - protein, 4 aa, synthetic construct
  REGION 1..4: note = Synthetic: Linker 4
GGAA

SEQ ID NO: 130 - protein, 5 aa, synthetic construct
  REGION 1..5: note = Synthetic: Linker 5
GGGGS

SEQ ID NO: 131 - protein, 5 aa, synthetic construct
  REGION 1..5: note = Synthetic: Linker 6
LAAAA

SEQ ID NO: 132 - protein, 4 aa, synthetic construct
  REGION 1..4: note = Synthetic: Linker 7
GGSG

SEQ ID NO: 133 - protein, 5 aa, synthetic construct
  REGION 1..5: note = Synthetic: Linker 8
GGSGG

SEQ ID NO: 134 - protein, 5 aa, synthetic construct
  REGION 1..5: note = Synthetic: Linker 9
GSGSG

SEQ ID NO: 135 - protein, 5 aa, synthetic construct
  REGION 1..5: note = Synthetic: Linker 10
GSGGG

SEQ ID NO: 136 - protein, 5 aa, synthetic construct
  REGION 1..5: note = Synthetic: Linker 11
GGGSG

SEQ ID NO: 137 - protein, 5 aa, synthetic construct
  REGION 1..5: note = Synthetic: Linker 12
GSSSG

SEQ ID NO: 138 - protein, 6 aa, synthetic construct
  REGION 1..6: note = Synthetic: N-terminal sequence 1
QGASTT

SEQ ID NO: 139 - protein, 6 aa, synthetic construct
  REGION 1..6: note = Synthetic: N-terminal sequence 2
QGASTS

SEQ ID NO: 140 - protein, 10 aa, synthetic construct
  REGION 1..10: note = Synthetic: Linker-IPV cleavage site
GSIPVSLRSG

SEQ ID NO: 141 - protein, 32 aa, synthetic construct
  REGION 1..32: note = Synthetic: VelBL24I/V28I/L31I
VDELQAEVDQLEDENYAIKTKIAQIRKKVEKL

SEQ ID NO: 142 - protein, 32 aa, synthetic construct
  REGION 1..32: note = Synthetic: VelA L24Y
VAQLEEKVKTLRAENYEYKSEVQRLEEQVAQL

SEQ ID NO: 143 - protein, 32 aa, synthetic construct
  REGION 1..32: note = Synthetic: VelA L24W
VAQLEEKVKTLRAENYEWKSEVQRLEEQVAQL

SEQ ID NO: 144 - protein, 32 aa, synthetic construct
  REGION 1..32: note = Synthetic: VelA L24F
VAQLEEKVKTLRAENYEFKSEVQRLEEQVAQL

SEQ ID NO: 145 - protein, 32 aa, synthetic construct
  REGION 1..32: note = Synthetic: VelA L24S
VAQLEEKVKTLRAENYESKSEVQRLEEQVAQL

SEQ ID NO: 146 - protein, 32 aa, synthetic construct
  REGION 1..32: note = Synthetic: VelA L24G
VAQLEEKVKTLRAENYEGKSEVQRLEEQVAQL

SEQ ID NO: 147 - protein, 32 aa, synthetic construct
  REGION 1..32: note = Synthetic: VelA L24V
VAQLEEKVKTLRAENYEVKSEVQRLEEQVAQL

SEQ ID NO: 148 - protein, 32 aa, synthetic construct; identical to SEQ ID NO: 57
  REGION 1..32: note = Synthetic: VelA L24I

SEQ ID NO: 149 - protein, 32 aa, synthetic construct
  REGION 1..32: note = Synthetic: VelA L24K
VAQLEEKVKTLRAENYEKKSEVQRLEEQVAQL

SEQ ID NO: 150 - protein, 32 aa, synthetic construct; identical to SEQ ID NO: 147
  REGION 1..32: note = Synthetic: VelA L24V

SEQ ID NO: 151 - protein, 32 aa, synthetic construct; identical to SEQ ID NO: 12
  REGION 1..32: note = Synthetic: VelA V28I

SEQ ID NO: 152 - protein, 32 aa, synthetic construct
  REGION 1..32: note = Synthetic: VelA V28L
VAQLEEKVKTLRAENYELKSELQRLEEQVAQL

SEQ ID NO: 153 - protein, 32 aa, synthetic construct
  REGION 1..32: note = Synthetic: VelA V28K
VAQLEEKVKTLRAENYELKSEKQRLEEQVAQL

SEQ ID NO: 154 - protein, 32 aa, synthetic construct
  REGION 1..32: note = Synthetic: VelA L24A/V28I
VAQLEEKVKTLRAENYEAKSEIQRLEEQVAQL

SEQ ID NO: 155 - protein, 32 aa, synthetic construct
  REGION 1..32: note = Synthetic: VelA L24A/V28L
VAQLEEKVKTLRAENYEAKSELQRLEEQVAQL

SEQ ID NO: 156 - protein, 32 aa, synthetic construct
  REGION 1..32: note = Synthetic: VelA L24R/L31V
VAQLEEKVKTLRAENYERKSEVQRVEEQVAQL

SEQ ID NO: 157 - protein, 32 aa, synthetic construct
  REGION 1..32: note = Synthetic: VelA V28A/L31E
VAQLEEKVKTLRAENYELKSEAQREEEQVAQL

SEQ ID NO: 158 - protein, 32 aa, synthetic construct
  REGION 1..32: note = Synthetic: VelA L24R
VAQLEEKVKTLRAENYERKSEVQRLEEQVAQL

SEQ ID NO: 159 - protein, 32 aa, synthetic construct
  REGION 1..32: note = Synthetic: VelA L24K/L31V
VAQLEEKVKTLRAENYEKKSEVQRVEEQVAQL

SEQ ID NO: 160 - protein, 32 aa, synthetic construct
  REGION 1..32: note = Synthetic: VelA V28S
VAQLEEKVKTLRAENYELKSESQRLEEQVAQL

SEQ ID NO: 161 - protein, 32 aa, synthetic construct
  REGION 1..32: note = Synthetic: VelA L31T
VAQLEEKVKTLRAENYELKSEVQRTEEQVAQL

SEQ ID NO: 162 - protein, 38 aa, synthetic construct
  REGION 1..38: note = Synthetic: VelA with amino-terminal QGASTT T5A
QGASATVAQLEEKVKTLRAENYELKSEVQRLEEQVAQL

SEQ ID NO: 163 - protein, 36 aa, synthetic construct
  REGION 1..36: note = Synthetic: VelA with amino-terminal QGASTT DT5/DT6
QGASVAQLEEKVKTLRAENYELKSEVQRLEEQVAQL

SEQ ID NO: 164 - protein, 38 aa, synthetic construct
  REGION 1..38: note = Synthetic: VelA with amino-terminal QGASTT T6A
QGASTAVAQLEEKVKTLRAENYELKSEVQRLEEQVAQL

SEQ ID NO: 165 - protein, 38 aa, synthetic construct
  REGION 1..38: note = Synthetic: VelA with amino-terminal QGASTT S4A
QGAATTVAQLEEKVKTLRAENYELKSEVQRLEEQVAQL

SEQ ID NO: 166 - protein, 38 aa, synthetic construct
  REGION 1..38: note = Synthetic: VelA with amino-terminal QGASTT T6S
QGASTSVAQLEEKVKTLRAENYELKSEVQRLEEQVAQL

SEQ ID NO: 167 - protein, 38 aa, synthetic construct
  REGION 1..38: note = Synthetic: VelA with amino-terminal QGASTT T6S/L24A
QGASTSVAQLEEKVKTLRAENYEAKSEIQRLEEQVAQL

SEQ ID NO: 168 - protein, 38 aa, synthetic construct
  REGION 1..38: note = Synthetic: VelA with amino-terminal QGASTT T6S/L24A/V28L
QGASTSVAQLEEKVKTLRAENYEAKSELQRLEEQVAQL

SEQ ID NO: 169 - protein, 32 aa, synthetic construct
  REGION 1..32: note = Synthetic: VelB L24G
VDELQAEVDQLEDENYAGKTKVAQLRKKVEKL

SEQ ID NO: 170 - protein, 32 aa, synthetic construct; identical to SEQ ID NO: 120
  REGION 1..32: note = Synthetic: VelB L24A

SEQ ID NO: 171 - protein, 32 aa, synthetic construct
  REGION 1..32: note = Synthetic: VelB L24Y
VDELQAEVDQLEDENYAYKTKVAQLRKKVEKL

SEQ ID NO: 172 - protein, 32 aa, synthetic construct
  REGION 1..32: note = Synthetic: VelB L24S
VDELQAEVDQLEDENYASKTKVAQLRKKVEKL

SEQ ID NO: 173 - protein, 32 aa, synthetic construct
  REGION 1..32: note = Synthetic: VelB L24F
VDELQAEVDQLEDENYAFKTKVAQLRKKVEKL

SEQ ID NO: 174 - protein, 32 aa, synthetic construct
  REGION 1..32: note = Synthetic: VelB L24W
VDELQAEVDQLEDENYAWKTKVAQLRKKVEKL

SEQ ID NO: 175 - protein, 32 aa, synthetic construct
  REGION 1..32: note = Synthetic: VelB L24V
VDELQAEVDQLEDENYAVKTKVAQLRKKVEKL

SEQ ID NO: 176 - protein, 32 aa, synthetic construct
  REGION 1..32: note = Synthetic: VelB L24K
VDELQAEVDQLEDENYAKKTKVAQLRKKVEKL

SEQ ID NO: 177 - protein, 32 aa, synthetic construct
  REGION 1..32: note = Synthetic: VelB V28L
VDELQAEVDQLEDENYALKTKLAQLRKKVEKL

SEQ ID NO: 178 - protein, 32 aa, synthetic construct
  REGION 1..32: note = Synthetic: VelB V28K
VDELQAEVDQLEDENYALKTKKAQLRKKVEKL

SEQ ID NO: 179 - protein, 32 aa, synthetic construct
  REGION 1..32: note = Synthetic: VelB L24I/V28I
VDELQAEVDQLEDENYAIKTKIAQLRKKVEKL

SEQ ID NO: 180 - protein, 32 aa, synthetic construct
  REGION 1..32: note = Synthetic: VelB L24V/V28I
VDELQAEVDQLEDENYAVKTKIAQLRKKVEKL

SEQ ID NO: 181 - protein, 32 aa, synthetic construct
  REGION 1..32: note = Synthetic: VelB L24I/V28L
VDELQAEVDQLEDENYAIKTKLAQLRKKVEKL

SEQ ID NO: 182 - protein, 32 aa, synthetic construct
  REGION 1..32: note = Synthetic: VelB L24S/L31Q
VDELQAEVDQLEDENYASKTKVAQQRKKVEKL

SEQ ID NO: 183 - protein, 32 aa, synthetic construct
  REGION 1..32: note = Synthetic: VelB L24H
VDELQAEVDQLEDENYAHKTKVAQLRKKVEKL

SEQ ID NO: 184 - protein, 32 aa, synthetic construct
  REGION 1..32: note = Synthetic: VelB V28S/L31W
VDELQAEVDQLEDENYALKTKSAQWRKKVEKL

SEQ ID NO: 185 - protein, 32 aa, synthetic construct
  REGION 1..32: note = Synthetic: VelB L31P
VDELQAEVDQLEDENYALKTKVAQPRKKVEKL

SEQ ID NO: 186 - protein, 44 aa, synthetic construct
  REGION 1..44: note = Synthetic: Vel light chain for CD studies
QGASTTVAQLEEKVKTLRAENYELKSEVQRLEEQVAQLGSIPVS

SEQ ID NO: 187 - protein, 44 aa, synthetic construct
  REGION 1..44: note = Synthetic: Vel heavy chain for CD studies
QGASTSVDELQAEVDQLEDENYALKTKVAQLRKKVEKLGSIPVS

SEQ ID NO: 188 - protein, 44 aa, synthetic construct
  REGION 1..44: note = Synthetic: Coil 10 light chain for CD studies
QGASTTVAQLEEKVKTLRAENYEAKSEVQRLEEQVAQLGSIPVS

SEQ ID NO: 189 - protein, 44 aa, synthetic construct
  REGION 1..44: note = Synthetic: Coil 10 heavy chain for CD studies
QGASTSVDELQAEVDQLEDENYAIKTKVAQLRKKVEKLGSIPVS

SEQ ID NO: 190 - protein, 44 aa, synthetic construct
  REGION 1..44: note = Synthetic: Coil 148 light chain for CD studies
QGASTTVAQLEEKVKTLRAENYEAKSELQRLEEQVAQLGSIPVS

SEQ ID NO: 191 - protein, 44 aa, synthetic construct
  REGION 1..44: note = Synthetic: Coil 148 heavy chain for CD studies
QGASTSVDELQAEVDQLEDENYAIKTKLAQLRKKVEKLGSIPVS

SEQ ID NO: 192 - protein, 8 aa, synthetic construct
  REGION 1..8: note = Synthetic: N-terminal residues of VelA with amino-terminal QGASTT
QGASTTVA

SEQ ID NO: 193 - protein, 8 aa, synthetic construct
  REGION 1..8: note = Synthetic: N-terminal modification 1
QGASATVA

SEQ ID NO: 194 - protein, 6 aa, synthetic construct
  REGION 1..6: note = Synthetic: N-terminal modification 2
QGASVA

SEQ ID NO: 195 - protein, 8 aa, synthetic construct
  REGION 1..8: note = Synthetic: N-terminal modification 3
QGASTAVA

SEQ ID NO: 196 - protein, 8 aa, synthetic construct
  REGION 1..8: note = Synthetic: N-terminal modification 4
QGAATTVA

SEQ ID NO: 197 - protein, 8 aa, synthetic construct
  REGION 1..8: note = Synthetic: N-terminal modification 5
QGASTSVA

SEQ ID NO: 198 - protein, 48 aa, synthetic construct
  REGION 1..48: note = Synthetic: VelA masking domain with amino-terminal QGASTT and linker IPV cleavage site
QGASTTVAQLEEKVKTLRAENYELKSEVQRLEEQVAQLGSIPVSLRSG

SEQ ID NO: 199 - protein, 48 aa, synthetic construct
  REGION 1..48: note = Synthetic: VelB masking domain with amino-terminal QGASTS and linker IPV cleavage site
QGASTSVDELQAEVDQLEDENYALKTKVAQLRKKVEKLGSIPVSLRSG

(19) United States
(12) Patent Application Publication
(10) Pub. No.: US 2024/0376200 A1
(43) Pub. Date: Nov. 14, 2024

(54) Improved Antibody Masking Domains

(71) Applicant: Seagen Inc., Bothell, WA (US)

(72) Inventor: Matthew Levengood, Bothell, WA (US)

(73) Assignee: Seagen Inc., Bothell, WA (US)

(21) Appl. No.: 18/691,970

(22) PCT Filed: Sep. 23, 2022
(86) PCT No.: PCT/US2022/076906

§ 371 Date: Mar. 14, 2024

Related U.S. Application Data
(60) Provisional application No. 63/248,122, filed on Sep. 24, 2021.

(51) Int. Cl. C07K 16/28; A61K 39/00; A61K 47/68; C07K 16/22; C07K 16/30

(52) U.S. Cl. CPC C07K 16/2803; A61K 47/68031; C07K 16/22; C07K 16/2809; C07K 16/2863; C07K 16/2866; C07K 16/2884; C07K 16/2887; C07K 16/2896; C07K 16/30; C07K 16/3084; A61K 2039/505; C07K 2319/30; C07K 2319/50

(57) ABSTRACT

Improved masking domains and masked antibodies comprising improved masking domains are provided. In some embodiments, masked antibodies comprising the improved masking domains demonstrate reduced aggregation. In various embodiments, masked antibodies comprising the improved masking domains may be used in therapeutic treatment.

### CROSS REFERENCE TO RELATED APPLICATIONS

This application claims the benefit of priority of U.S. Provisional Application No. 63/248,122, filed Sep. 24, 2021, which is incorporated by reference herein in its entirety for any purpose.

### FIELD OF THE INVENTION

The present invention relates to the field of masked antibodies comprising coiled-coil masking domains. In particular, the present invention relates to masked antibodies with reduced aggregation.

### BACKGROUND

Current antibody-based therapeutics may have less than optimal selectivity for the intended target. Although monoclonal antibodies are typically specific for binding to their intended targets, most target molecules are not specific to the disease site and may be present in cells or tissues other than the disease site.

Several approaches have been described for overcoming these off-target effects by engineering antibodies to have a cleavable linker attached to an inhibitory or masking domain that inhibits antibody binding (see, e.g., WO2003/068934, WO2004/009638, WO 2009/025846, WO2101/081173 and WO2014103973). The linker can be designed to be cleaved by enzymes that are specific to certain tissues or pathologies, thus enabling the antibody to be preferentially activated in desired locations. Masking moieties can act by binding directly to the binding site of an antibody or can act indirectly via steric hindrance. Various masking moieties, linkers, protease sites and formats of assembly have been proposed. The extent of masking may vary between different formats as may the compatibility of masking moieties with expression, purification, conjugation, or pharmacokinetics of antibodies.

The present invention relates to masked antibodies with reduced aggregation. In some embodiments, the masked antibodies comprise a first coiled-coil domain linked to a heavy chain variable region of the antibody and a second coiled-coil domain linked to a light chain variable region of the antibody. The presence of these potentially hydrophobic coiled-coil polypeptide sequences can lead to aggregation. In some embodiments, the improved masking domains described herein demonstrate reduced aggregation of masked antibodies comprising the improved masking domains.

### SUMMARY

The present disclosure relates masked antibodies that comprise an improved removable masking agent (e.g., a coiled coil masking agent) that prevents binding of the antibodies to their intended targets until the masking agent is cleaved off or otherwise removed. In other words, the masking agent masks the antigen binding portion of the antibody so that it cannot interact with its targets. In certain therapeutic uses, the masking agent can be removed (e.g., cleaved) by one or more molecules (e.g., proteases) that are present in an in vivo environment after administration of the masked antibody to a patient. In other uses, for example, non-therapeutic uses, a masking agent could be removed by adding one or more proteases to the medium in which the antibody is being used. Removal of the masking agent restores the ability of the antibodies to bind to their targets, thus enabling specific targeting of the antibodies.

In some embodiments, masked antibodies comprising the improved masking domains provided herein demonstrate reduced aggregation compared to masked antibodies comprising masking domains lacking the improvements.

In some embodiments, a masked antibody is provided. The following non-limiting embodiments are provided.
Embodiment 1. A masked antibody comprising a first masking domain comprising a first coiled-coil domain and a second masking domain comprising a second coiled-coil domain, wherein the first masking domain or second masking domain is linked to a heavy chain of an antibody and the other of the first masking domain or second masking domain is linked to a light chain of the antibody, wherein the first coiled-coil domain comprises the sequence
   V₇D₈E₉L₁₀Q₁₁A₁₂E₁₃V₁₄D₁₅Q₁₆L₁₇E₁₈D₁₉E₂₀N₂₁Y₂₂A₂₃L₂₄K₂₅T₂₆K₂₇V₂₈A₂₉Q₃₀L₃₁R₃₂K₃₃ K₃₄V₃₅ E₃₆K₃₇L₃₈ (SEQ ID NO: 2), and the second coiled-coil domain comprises the sequence
   V₇A₈Q₉L₁₀E₁₁E₁₂K₁₃V₁₄K₁₅T₁₆L₁₇R₁₈A₁₉E₂₀N₂₁Y₂₂E₂₃L₂₄K₂₅S₂₆E₂₇V₂₈Q₂₉R₃₀L₃₁E₃₂E₃₃Q₃₄V₃₅A₃₆Q₃₇L₃₈ (SEQ ID NO: 1); wherein the first coiled-coil domain and/or the second coiled-coil domain comprises at least one amino acid substitution in SEQ ID NO: 2 or SEQ ID NO: 1 that reduces aggregation of the masked antibody in an aqueous formulation compared to the masked antibody without the at least one amino acid substitution in the same aqueous formulation.
Embodiment 2. The masked antibody of embodiment 1, wherein the at least one amino acid substitution reduces aggregation of the masked antibody in an aqueous formulation at pH6-8.5, pH 6.5, 6.6, 6.7, 6.8, 6.9, 7, 7.1, 7.2, 7.3, 7.4, 7.5, 7.6, 7.7, and/or 7.8.
Embodiment 3. The masked antibody of embodiment 1 or embodiment 2, wherein at least one amino acid substitution replaces an acidic amino acid with a non-acidic amino acid.
Embodiment 4. The masked antibody of embodiment 3, wherein at least one acidic amino acid is aspartic acid or glutamic acid.
Embodiment 5. The masked antibody of embodiment 3 or embodiment 4, wherein the non-acidic amino acid is any amino acid other than aspartic acid or glutamic acid.
Embodiment 6. The masked antibody of embodiment 5, wherein at least one acidic amino acid is replaced with a non-acidic amino acid selected from asparagine, glutamine, lysine, histidine, arginine, serine, phenylalanine, tyrosine, tryptophan, threonine, leucine, isoleucine, and methionine.
Embodiment 7. The masked antibody of embodiment 6, wherein at least one acidic amino acid is replaced with a non-acidic amino acid selected from asparagine, glutamine, lysine, histidine, arginine, and serine.
Embodiment 8. The masked antibody of any one of embodiments 3-7, wherein one, two, three, or four acidic amino acids in the first coiled-coil domain and/or one, two, three, or four acidic amino acids in the second coiled-coil domain are replaced with non-acidic amino acids.
Embodiment 9. The masked antibody of any one of embodiments 3-8, wherein at least one acidic amino acid selected from D8, E9, E13, D15, D19, and E36 in the first coiled-coil domain is replaced with a non-acidic amino acid.
Embodiment 10. The masked antibody of any one of embodiments 3-9, wherein at least one acidic amino acid selected from E11, E12, E20, E23, E32, and E33 in the second coiled-coil domain is replaced with a non-acidic amino acid.
Embodiment 11. The masked antibody of any one of embodiments 1-10, wherein the first coiled-coil domain comprises amino acid substitutions at D8 and E36.
Embodiment 12. The masked antibody of embodiment 11, wherein the first coiled-coil domain comprises amino acid substitutions D8K and E36H.
Embodiment 13. The masked antibody of any one of embodiments 1-12, wherein the first coiled-coil domain comprises a first substitution of a first hydrophobic amino acid with a less bulky hydrophobic amino acid or a more bulky hydrophobic amino acid, and the second coiled-coil domain comprises a second substitution of a second hydrophobic amino acid with a less bulky hydrophobic amino acid or a more bulky hydrophobic amino acid, wherein one coiled-coil domain comprises the less bulky hydrophobic amino acid substitution and one coiled-coil domain comprises the more bulky hydrophobic amino acid substitution, wherein the first hydrophobic amino acid and the second hydrophobic amino acid are at the same amino acid position in SEQ ID NOs: 2 and 1, respectively.
Embodiment 14. The masked antibody of embodiment 13, wherein the first hydrophobic amino acid and the second hydrophobic amino acid are both valine or leucine.
Embodiment 15. The masked antibody of embodiment 13 or embodiment 14, wherein the less bulky hydrophobic amino acid is alanine, glycine, or serine.
Embodiment 16. The masked antibody of any one of embodiments 13-15, wherein the more bulky hydrophobic amino acid is isoleucine, phenylalanine, tyrosine, tryptophan, or methionine.
Embodiment 17. The masked antibody of any one of embodiments 13-16, comprising wherein the first coiled-coil and the second coiled-coil comprise a substitution at position 24 and/or at position 28.
Embodiment 18. The masked antibody of any one of embodiments 13-17, wherein one, two, three, or four pairs of hydrophobic amino acids are substituted.
Embodiment 19. The masked antibody of any one of embodiments 13-18, wherein the first coiled-coil domain and the second coiled-coil domain comprise at least one pair of substitutions at at least one pair of positions selected from V14/V14, L17/L17, L24/L24, V28/V28, L31/L31, and V35/V35, wherein the first position is the position in the first coiled-coil domain and the second position is the position in the second coiled-coil domain.
Embodiment 20. The masked antibody of embodiment 19, wherein the first coiled-coil domain and the second coiled-coil domain comprise at least one pair of substitutions selected from V14A/V14I, V14I/V14A, L17A/L17I, L17I/L17A, L24A/L24I, L24I/L24A, L24G/L24Y, L24Y/L24G, L24A/L24Y, L24Y/L24A, L24Y/L24W, L24W/L24Y, L24Y/L24F, L24F/L24Y, L24S/L24F, L24F/L24S, L24Y/L24Y, L24S/L24S, L24G/L24W, L24W/L24G, L24G/L24F, L24F/L24G, L24A/L24W, L24W/L24A, L24W/L24F, L24F/L24W, L24A/L24F, L24F/L24A, L24W/L24S, L24S/L24W, L24Y/L24S, L24S/L24Y, L24F/L24F, L24I/L24V, L24V/L24I, L24K/L24I, L24I/L24K, V28A/V28I, V28I/V28A, V28N/V28N, V28L/V28I, V28I/V28L, V28K/V28I, V28I/V28K, V28L/V28L, L31A/L31I, L31I/L31A, V35A/V35I, V35I/V35A, and V35N/V35N.
Embodiment 21. The masked antibody of embodiment 20, wherein the first coiled-coil domain and the second coiled-coil domain comprise one pair of substitutions selected from: L24A/L24I, L24I/L24A, L24V/L24A, L24A/L24V, V28A/V28I, V28I/V28A, V28L/V28L, L31A/L31I, or L31I/L31A.
Embodiment 22. The masked antibody of embodiment 20, wherein the first coiled-coil domain and the second coiled-coil domain comprise two pairs of substitutions, wherein the first pair of substitutions is selected from L24A/L24I and L24I/L24A, and the second pair of substitutions is selected from L31A/L311 and L31I/L31A;
   or wherein the first pair of substitutions is selected from L17A/L17I and L17I/L17A, and the second pair of substitutions is selected from L31A/L311 and L31I/L31A;
   or wherein the first pair of substitutions is selected from L17A/L171 and L17I/L17A, and the second pair of substitutions is selected from L24A/L24I and L24I/L24A;
   or wherein the first pair of substitutions is selected from V28A/V281 and V28I/V28A, and the second pair of substitutions is selected from L31A/L311 and L31I/L31A;
   or wherein the first pair of substitutions is selected from L24V/L24A and L24A/L24V, and the second pair of substitutions is V28L/V28L;
   or wherein the first pair of substitutions is selected from L24I/L24A and L24A/L24I, and the second pair of substitutions is V28L/V28L.
Embodiment 23. The masked antibody of embodiment 20, wherein the first coiled-coil domain and the second coiled-coil domain comprise three pairs of substitutions, wherein the first pair of substitutions is selected from L17A/L171 and L17I/L17A, the second pair of substitutions is selected from L24A/L24I and L24I/L24A, and the third pair of substitutions is selected from L31A/L311 and L31I/L31A.
Embodiment 24. The masked antibody of any one of embodiments 1-23, wherein:
   the first coiled-coil domain comprises substitutions L24A and L31A, and the second coiled-coil domain comprises substitutions L24I and L31I; or
   the first coiled-coil domain comprises substitutions L24I and L31I, and the second coiled-coil domain comprises substitutions L24A and L31A; or
   the first coiled-coil domain comprises substitutions L171 and L31I, and the second coiled-coil domain comprises substitutions L17A and L31A; or
   the first coiled-coil domain comprises substitutions L17I, L24I, and L31I, and the second coiled-coil domain comprises substitutions L17A, L24A, and L31A; or
   the first coiled-coil domain comprises substitutions V281 and L31I, and the second coiled-coil domain comprises substitutions V28A and L31A; or
   the first coiled-coil domain comprises substitution L31I, and the second coiled-coil domain comprises substitutions L31A and E33K; or
   the first coiled-coil domain comprises substitutions D8K and E36H, and the second coiled-coil domain comprises no substitutions; or
   the first coiled-coil domain comprises substitutions D8K, E36H, and L31I, and the second coiled-coil domain comprises substitution L31A; or
   the first coiled-coil domain comprises substitutions D8K, D15K, and L31I, and the second coiled-coil domain comprises substitution L31A; or
   the first coiled-coil domain comprises substitutions D15K, E36H, and L31I, and the second coiled-coil domain comprises substitution L31A.
Embodiment 25. The masked antibody of any one of embodiments 1-23, wherein:
   the first coiled-coil domain comprises a substitution L24V, and the second coiled-coil domain comprises a substitution L24A; or
   the first coiled-coil domain comprises a substitution L24A, and the second coiled-coil domain comprises a substitution L24V; or
   the first coiled-coil domain comprises substitutions L24V and V28I, and the second coiled-coil domain comprises a substitution L24A; or
   the first coiled-coil domain comprises a substitution L24A, and the second coiled-coil domain comprises substitutions L24V and V28I; or
   the first coiled-coil domain comprises substitutions L241 and V28L, and the second coiled-coil domain comprises substitutions L24A and V28L; or
   the first coiled-coil domain comprises substitutions L24A and V28L, and the second coiled-coil domain comprises substitutions L241 and V28L; or
   the first coiled-coil domain comprises substitutions L24V and V28I, and the second coiled-coil domain comprises substitutions T6S and L24A; or
   the first coiled-coil domain comprises substitutions T6S and L24A, and the second coiled-coil domain comprises substitutions L24V and V28I; or
   the first coiled-coil domain comprises substitutions L241 and V28L, and the second coiled-coil domain comprises substitutions T6S, L24A and V28L; or
   the first coiled-coil domain comprises substitutions T6S, L24A and V28L, and the second coiled-coil domain comprises substitutions L241 and V28L.
Embodiment 26. The masked antibody of any one of embodiments 1-25, wherein the first coiled-coil domain comprises a sequence selected from SEQ ID NOs: 2, 59-121, 141, and 169-185.
Embodiment 27. The masked antibody of any one of embodiments 1-26, wherein the second coiled-coil domain comprises a sequence selected from SEQ ID NOs: 1, 5-58, 142-168.
Embodiment 28. The masked antibody of any one of embodiments 1-27, wherein the first coiled-coil domain and second coiled-coil domain comprise the sequences of SEQ ID NOs: 66 and 11, respectively; or SEQ ID NOs: 70 and 15, respectively; or SEQ ID NOs: 72 and 17, respectively; or SEQ ID NOs: 94 and 1, respectively; or SEQ ID NOs: 108 and 1, respectively; or SEQ ID NOs: 108 and 11, respectively; or SEQ ID NOs: 110 and 49, respectively; or SEQ ID NOs: 111 and 50, respectively; or SEQ ID NOs: 112 and 51, respectively; or SEQ ID NOs: 113 and 52, respectively; or SEQ ID NOs: 114 and 53, respectively; or SEQ ID NOs: 70 and 56, respectively; or SEQ ID NOs: 116 and 1, respectively; or SEQ ID NOs: 117 and 15, respectively; or SEQ ID NOs: 118 and 15, respectively; or SEQ ID NOs: 119 and 15, respectively; or SEQ ID NOs: 120 and 57, respectively; or SEQ ID NOs: 121 and 57, respectively; or SEQ ID NOs: 63 and 8, respectively.
Embodiment 29. The masked antibody of any one of embodiments 1-27, wherein the first coiled-coil domain and second coiled-coil domain comprise the sequences of SEQ ID NOs: 181 and 168, respectively; or SEQ ID NOs: 180 and 167, respectively; or SEQ ID NOs: 181 and 155, respectively; or SEQ ID NOs: 180 and 11, respectively.
Embodiment 30. The masked antibody of any one of embodiments 1-29, wherein each masking domain comprises an amino-terminal sequence selected from SEQ ID NOs: 138 and 139.
Embodiment 31. The masked antibody of embodiment 30, wherein the first masking domain comprises the amino-terminal sequence of SEQ ID NO: 139 and the second masking domain comprises the amino-terminal sequence of SEQ ID NO: 138.
Embodiment 32. The masked antibody of any one of embodiments 1-30, wherein each masking domain comprises an amino-terminal sequence of SEQ ID NO: 139.
Embodiment 33. A masked antibody comprising a first masking domain comprising a first coiled-coil domain and a second masking domain comprising a second coiled-coil domain, wherein the first masking domain or second masking domain is linked to a heavy chain of an antibody and the other of the first masking domain or second masking domain is linked to a light chain of the antibody, wherein the first coiled-coil domain and/or the second coiled-coil domain comprises at least one amino acid substitution that reduces aggregation of the masked antibody in an aqueous formulation compared to the masked antibody without the at least one amino acid substitution in the same aqueous formulation.
Embodiment 34. The masked antibody of embodiment 33, wherein the at least one amino acid substitution reduces aggregation of the masked antibody in an aqueous formulation at pH6-8.5, pH 6.5, 6.6, 6.7, 6.8, 6.9, 7, 7.1, 7.2, 7.3, 7.4, 7.5, 7.6, 7.7, and/or 7.8.
Embodiment 35. The masked antibody of embodiment 33 or embodiment 34, wherein at least one amino acid substitution reduces homodimerization of the first masking domain and/or the second masking domain.
Embodiment 36. The masking domain of any one of embodiments 33-35, wherein at least one amino acid substitution increases heterodimerization of the first masking domain and the second masking domain.
Embodiment 37. The masked antibody of any one of embodiments 33-36, wherein at least one amino acid substitution replaces an acidic amino acid in the first coiled coil domain or the second coiled coil domain with a non-acidic amino acid.
Embodiment 38. The masked antibody of embodiment 37, wherein at least one acidic amino acid is aspartic acid or glutamic acid.
Embodiment 39. The masked antibody of embodiment 37 or embodiment 38, wherein the non-acidic amino acid is any amino acid other than aspartic acid or glutamic acid.
Embodiment 40. The masked antibody of embodiment 39, wherein at least one acidic amino acid is replaced with a non-acidic amino acid selected from asparagine, glutamine, lysine, histidine, arginine, serine, phenylalanine, tyrosine, tryptophan, threonine, leucine, isoleucine, and methionine.
Embodiment 41. The masked antibody of embodiment 40, wherein at least one acidic amino acid is replaced with a non-acidic amino acid selected from asparagine, glutamine, lysine, histidine, arginine, and serine.
Embodiment 42. The masked antibody of any one of embodiments 37-41, wherein one, two, three, or four acidic amino acids in the first coiled-coil domain and/or one, two, three, or four acidic amino acids in the second coiled-coil domain are replaced with non-acidic amino acids.
Embodiment 43. The masked antibody of any one of embodiments 33-42, wherein the first coiled-coil domain comprises a first substitution of a first hydrophobic amino acid with a less bulky hydrophobic amino acid or a more bulky hydrophobic amino acid, and the second coiled-coil domain comprises a second substitution of a second hydrophobic amino acid with a less bulky hydrophobic amino acid or a more bulky hydrophobic amino acid, wherein one coiled-coil domain comprises the less bulky hydrophobic amino acid substitution and one coiled-coil domain comprises the more bulky hydrophobic amino acid substitution, wherein the first hydrophobic amino acid and the second hydrophobic amino acid are at corresponding amino acid positions in the first coiled-coil domain and second coiled-coil domain, respectively.
Embodiment 44. The masked antibody of embodiment 43, wherein the first hydrophobic amino acid and the second hydrophobic amino acid are both valine or leucine.
Embodiment 45. The masked antibody of embodiment 43 or embodiment 44, wherein the less bulky hydrophobic amino acid is alanine, glycine, or serine.
Embodiment 46. The masked antibody of any one of embodiments 43-45, wherein the more bulky hydrophobic amino acid is isoleucine, phenylalanine, tyrosine, tryptophan, or methionine.
Embodiment 47. The masked antibody of any one of embodiments 43-46, wherein one, two, three, or four pairs of hydrophobic amino acids are substituted.
Embodiment 48. The masked antibody of any one of embodiments 1-47, wherein each masking domain comprises a protease-cleavable linker and is linked to the heavy chain or light chain via the protease-cleavable linker.
Embodiment 49. The masked antibody of embodiment 48, wherein the protease-cleavable linker comprises a matrix metalloprotease (MMP) cleavage site, a urokinase plasminogen activator cleavage site, a matriptase cleavage site, a legumain cleavage site, a Disintegrin and Metalloprotease (ADAM) cleavage site, or a caspase cleavage site.
Embodiment 50. The masked antibody of embodiment 49, wherein the protease-cleavable linker comprises a MMP cleavage site.
Embodiment 51. The masked antibody of embodiment 50, wherein the MMP cleavage site is selected from an MMP2 cleavage site, an MMP7 cleavage site, an MMP9 cleavage site and an MMP13 cleavage site.
Embodiment 52. The masked antibody of embodiment 50 or embodiment 51, wherein the MMP cleavage site comprises the sequence IPVSLRSG (SEQ ID NO: 122) or GPLGVR (SEQ ID NO: 123).
Embodiment 53. The masked antibody of any one of embodiments 50-52, wherein each masking domain comprises a protease-cleavable linker of SEQ ID NO: 140.
Embodiment 54. The masked antibody of any one of c embodiments 1-53, wherein the first masking domain is linked to the amino-terminus of the heavy chain and the second masking domain is linked to the amino-terminus of the light chain.
Embodiment 55. The masked antibody of any one of embodiments 1-53, wherein the first masking domain is linked to the amino-terminus of the light chain and the second masking domain is linked to the amino-terminus of the heavy chain.
Embodiment 56. The masked antibody of any one of embodiments 1-55, wherein the antibody binds a therapeutic antigen.
Embodiment 57. The masked antibody of embodiment 56, wherein the antibody is useful for treating cancer, an autoimmune disorder, or an infection.
Embodiment 58. The masked antibody of any one of embodiments 1-56, wherein the antibody binds a tumor-associated antigen.
Embodiment 59. The masked antibody of embodiment 58, wherein the tumor-associated antigen is selected from CD47, CD3, CD19, CD20, CD22, CD30, CD33, CD34, CD40, CD44, CD52, CD70, CD79a, CD123, Her-2, EphA2, lymphocyte associated antigen 1, VEGF or VEGFR, CTLA-4, LIV-1, nectin-4, CD74, SLTRK-6, EGFR, CD73, PD-L1, CD163, CCR4, CD147, EpCam, Trop-2, CD25, C5aR, Ly6D, alpha v integrin, B7H3, B7H4, Her-3, folate receptor alpha, GD-2, CEACAM5, CEACAM6, c-MET, CD266, MUC1, CD10, MSLN, sialyl Tn, Lewis Y, CD63, CD81, CD98, CD166, tissue factor (CD142), CD55, CD59, CD46, CD164, TGF beta receptor 1 (TGFBR1), TGFBR2, TGFBR3, FasL, MerTk, Axl, Clec12A, CD352, FAP, CXCR3, and CD5.
Embodiment 60. The masked antibody of any one of embodiments 1-59, wherein the masked antibody has reduced binding affinity for the antibody's target antigen compared to the binding affinity of the antibody for its target antigen without the first masking domain and second masking domain.
Embodiment 61. The masked antibody of embodiment 60, wherein the binding affinity of the masked antibody is reduced at least about 100-fold compared to the binding affinity of the antibody without the first masking domain and second masking domain.
Embodiment 62. The masked antibody of embodiment 61, wherein the binding affinity of the masked antibody is reduced between about 200-fold and about 1500-fold compared to the binding affinity of the antibody without the first masking domain and second masking domain.
Embodiment 63. The masked antibody of any one of embodiments 1-62, wherein the aqueous formulation comprises 10-100 mM of a buffering agent and 10-100 mM salt.
Embodiment 64. The masked antibody of embodiment 63, wherein the buffering agent comprises potassium phosphate and/or sodium phosphate and the salt comprises NaCl and/or KCl.
Embodiment 65. The masked antibody of embodiment 64, wherein the aqueous formulation is PBS.
Embodiment 66. The masked antibody of any one of embodiments 1-65, wherein the aqueous formulation comprises 1-20 mg/mL, 1-15 mg/mL, 1-10 mg/mL, 1-5 mg/mL, or 1-3 mg/mL masked antibody.
Embodiment 67. An immunoconjugate comprising the masked antibody of any one of embodiments 1-66 and a cytotoxic agent.
Embodiment 68. The immunoconjugate of embodiment 67, wherein the cytotoxic agent is an antitubulin agent, a DNA minor groove binding agent, a DNA replication inhibitor, a DNA alkylator, a topoisomerase inhibitor, a NAMPT inhibitor, or a chemotherapy sensitizer.
Embodiment 69. The immunoconjugate of embodiment 67 or embodiment 68, wherein the cytotoxic agent is an anthracycline, an auristatin, a camptothecin, a duocarmycin, an etoposide, an enediyine antibiotic, a lexitropsin, a taxane, a maytansinoid, a pyrrolobenzodiazepine, a combretastatin, a cryptophysin, or a vinca alkaloid.
Embodiment 70. The immunoconjugate of any one of embodiments 67-69, wherein the cytotoxic agent is auristatin E, AFP, AEB, AEVB, MMAF, MMAE, paclitaxel, docetaxel, doxorubicin, morpholino-doxorubicin, cyanomorpholino-doxorubicin, melphalan, methotrexate, mitomycin C, a CC-1065 analogue, CBI, calicheamicin, maytansine, an analog of dolastatin 10, rhizoxin, or palytoxin, epothilone A, epothilone B, nocodazole, colchicine, colcimid, estramustine, cemadotin, discodermolide, eleutherobin, a tubulysin, a plocabulin, or maytansine.
Embodiment 71. The immunoconjugate of embodiment 70, wherein the cytotoxic agent is an auristatin.
Embodiment 72. The immunoconjugate of embodiment 71, wherein the cytotoxic agent is MMAE or MMAF.
Embodiment 73. A nucleic acid sequence encoding the masked antibody of any one of embodiments 1-66.
Embodiment 74. An expression vector comprising the nucleic acid of embodiment 73.
Embodiment 75. A host cell comprising the nucleic acid of embodiment 73 or the expression vector of embodiment 70.
Embodiment 76. A host cell that expresses the masked antibody of any one of embodiments 1-66.
Embodiment 77. A method of producing the masked antibody of any one of embodiments 1-66 comprising culturing the host cell of embodiment 75 or embodiment 76 under conditions suitable for expressing the masked antibody.
Embodiment 78. The method of embodiment 77, further comprising isolating the masked antibody.
Embodiment 79. A pharmaceutical composition comprising the masked antibody of any one of embodiments 1-66 or the immunoconjugate of any one of embodiments 67-72 and a pharmaceutically acceptable carrier.
Embodiment 80. The masked antibody of any one of embodiments 1-66, the immunoconjugate of any one of embodiments 67-72, or the pharmaceutical composition of embodiment 79 for use in therapy.
Embodiment 81. The masked antibody of any one of embodiments 1-66, the immunoconjugate of any one of embodiments 67-72, or the pharmaceutical composition of embodiment 79 for use in treating cancer, an autoimmune disease, or an infection.
Embodiment 82. Use of the masked antibody of any one of embodiments 1-66, the immunoconjugate of any one of embodiments 67-72, or the pharmaceutical composition of embodiment 79 for the preparation of a medicament for use in therapy.
Embodiment 83. Use of the masked antibody of any one of embodiments 1-66, the immunoconjugate of any one of embodiments 67-72, or the pharmaceutical composition of embodiment 79 for the preparation of a medicament for use in treating cancer, an autoimmune disease, or an infection.
Embodiment 84. A method comprising administering to a subject in need thereof the masked antibody of any one of embodiments 1-66, the immunoconjugate of any one of embodiments 67-72, or the pharmaceutical composition of embodiment 79.
Embodiment 85. A method of treating cancer, an autoimmune disease, or an infection comprising administering to a subject in need thereof the masked antibody of any one of embodiments 1-66, the immunoconjugate of any one of embodiments 67-72, or the pharmaceutical composition of embodiment 79.
Embodiment 86. The method of embodiment 85, which is a method of treating cancer.
Embodiment 87. The method of embodiment 86, wherein the cancer is a solid cancer, a soft tissue cancer, or a hematopoietic cancer.
Embodiment 88. The method of embodiment 86 or embodiment 87, wherein the cancer is selected from lung cancer, pancreatic cancer, breast cancer, liver cancer, ovarian cancer, testicular cancer, kidney cancer, bladder cancer, spinal cancer, brain cancer, cervical cancer, endometrial cancer, colorectal cancer, anal cancer, endometrial cancer, esophageal cancer, gallbladder cancer, gastrointestinal cancer, gastric cancer, sarcoma, head and neck cancer, melanoma, skin cancer, prostate cancer, pituitary cancer, stomach cancer, uterine cancer, vaginal cancer, thyroid cancer, a sarcoma, soft tissue sarcoma, osteosarcoma, a lymphoma, diffuse large B-cell lymphomas (DLBCL), follicular lymphoma, myelodysplastic syndrome (MDS), Hodgkin's disease, a malignant lymphoma, non-Hodgkin's lymphoma, Burkitt's lymphoma, multiple myeloma, Richter's Syndrome, a leukemia, acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia (T-PLL), large granular lymphocytic leukemia, adult T-cell leukemia, and acute monocytic leukemia (AMOL).

The summary of the disclosure described above is non-limiting, and other features and advantages of the disclosed antibodies and methods of making and using them will be apparent from the following drawings, the detailed description, the examples and the claims.

### BRIEF DESCRIPTION OF THE DRAWINGS

FIG. 1 provides analytical size-exclusion chromatography (SEC) data for the percentage high molecular weight (%) of Vel-IPV-Ab1 antibody at 0 hours or after a 96-hour incubation at room temperature.

FIG. 2 shows pH-dependence of Vel-IPV-Ab1 aggregation as measured by percentage aggregate after a 96-hour incubation at room temperature.

FIG. 3 shows aggregation of Vel-IPV-Ab1 at different NaCl concentration in sodium acetate buffer at pH 4.5 after a 96-hour incubation at room temperature.

FIG. 4 shows aggregation of Vel-IPV-Ab1 at different NaCl concentration in HEPES buffer at pH 7.5 after a 96-hour incubation at room temperature.

FIGS. 5A-5C show normalized analytical SEC overlay of control, unmasked Ab1 antibody plus matrix metalloprotease 2 (MMP-2) (A), masked Vel-IPV-Ab1 without demasking (B), and masked Vel-IPV-Ab1 demasked with MMP-2 treatment (C). HMW refers to the presence of high molecular weight species.

FIGS. 6A-6B show HMW peaks and percentage HMW species for unconjugated Vel-IPV-Ab1 (A) or a Vel-IPV-Ab1 antibody-drug conjugate (ADC).

FIG. 7 shows heavy chain (VelB; SEQ ID NO: 199) and light chain (VelA; SEQ ID NO: 198) sequences of Vel-IPV coiled-coil masking domain. The sequences of the coiled-coil domain of VelA (SEQ ID NO: 1) or of the coiled-coil domain VelB (SEQ ID NO: 2) are also shown. The heptad repeats of the Vel coiled-coil sequence are noted from the a-g positions, as are the sequence of the helix cap and the protease-sensitive linker sequence (SEQ ID NO: 140) that adjoins the mask to the antibody of interest.

FIG. 8 provides analytical SEC data for the percentage high molecular weight (%) Vel-IPV-Ab1 Coil 10 bearing light chain L24A and heavy chain L24I mutations in the coiled-coil domain at high concentration. Experiments were performed at 0 hours or after a 7 day incubation in PBS at room temperature.

FIG. 9 shows saturation binding ELISA results for Ab1 (unmasked parent), Vel-IPV-Ab1 (Vel), and several Vel variants (mutations described in Table 2) against recombinant Ab1 target.

FIG. 10 shows saturation binding ELISA results for Ab1 (unmasked), Vel-IPV-Ab1 (Vel), and Vel-IPV-Ab1 Coil 10 and Coil 14 (mutations described in Table 2) against recombinant Ab1 target. Antibodies were expressed in either CHO or HEK cells.

FIG. 11 shows saturation binding ELISA results for Ab1 (unmasked), Vel-IPV-Ab1 (Vel), and Vel-IPV-Ab1 Coils 91, 92, 95, and 96 (mutations described in Table 5) against recombinant Ab1 target.

FIG. 12 shows saturation binding ELISA results for Ab1 (unmasked), Vel-IPV-Ab1 (Vel), and Vel-IPV-Ab1 Coils 88-90 (mutations described in Table 5) against recombinant Abl target.

FIG. 13 shows saturation binding ELISA results for Ab1 (unmasked), Vel-IPV-Ab1 (Vel), and Vel-IPV-Ab1 Coils 97-99 (mutations described in Table 5) against recombinant Ab1 target.

FIG. 14 shows saturation binding FACS results with Ab1 target-expressing HEK-293F cells for Ab1, Vel-IPV-Ab1, and Vel-IPV-Ab1 Coil 10 and Coil 14 (described in Table 2).

FIG. 15 shows cleavage of masks for different Vel-IPV-Ab1 variants as measured by LC-MS.

FIG. 16 shows in vivo efficacy in the HPAF-II xenograft model for Vel-IPV-Ab1 MDpr-gluc-PEG12-MMAE (8) ADCs.

FIG. 17 shows in vivo efficacy in the BxPC3 xenograft model for Vel-IPV-Ab1 MDpr-gluc-PEG12-MMAE (8) ADCs.

FIG. 18 shows saturation binding ELISA results for hB6H12.3, Vel-IPV-hB6H12.3, and several Vel variants against recombinant human CD47. The L17I/L17A (L17), L24I/L24A (L24), L31I/L31A (L31), and VelA/VelB switch masked hB6H12.3 antibodies are described in Table 8. Unmasked=unmasked antibody; Vel=wild-type Vel mask.

FIG. 19 shows saturation binding ELISA results for hB6H12.3, Vel-IPV-hB6H12.3, and several additional Vel variants against recombinant human CD47. L24/L31 is antibody with a Vel mask variant with heavy chain mutations of L24I/L311 and light chain mutations of L24A/L31A as described in Table 8. D8K and E36H refer to heavy chain mutations in Vel.

FIG. 20 shows saturation binding flow cytometry results for hB6H12.3, Vel-IPV-hB6H12.3, and several Vel variants on CD47 (+) Raji cells.

FIG. 21 shows saturation binding ELISA results for rituximab, Vel-IPV-rituximab, and several Vel variants against recombinant human CD20. Rituximab masked with Vel variants are described in Table 10. Vel-rituximab=rituximab masked with wildtype Vel.

FIG. 22 shows saturation binding ELISA for trastuzumab, Vel-IPV-trastuzumab, and several Vel variants against recombinant human HER2. Trastuzumab masked with Vel variants are described in Table 9. Vel-trastuzumab=trastuzumab masked with wildtype Vel.

FIG. 23 shows in vivo efficacy in the Detroit563 xenograft model for hB6H12.3 with different Vel masks. q4dx4=dosing every four days for four doses.

FIG. 24 shows circular dichroism experiments with VelA (light chain) peptide and L24A and L31A VelA mutants.

FIG. 25 shows circular dichroism studies to determine the relative heterodimeric affinities of the VelA and VelB peptides.

FIG. 26A-26B show in vivo plasma stability of Vel-IPV-Ab1, Vel-scr-Ab1, and Coil 10 masks of these (mutations as in Table 2). FIG. 26A shows the extent of cleavage of the heavy chain peptide at 48 hours post-dose. FIG. 26B shows the Western blot at 1 hour, 6 hour, 24 hours, 48 hours, and 96 hours. Cleavage of the Coil 10 mask was observed irrespective of cleavage sequence used. (IPV=MMP-cleavable; scr=MMP non-cleavable).

FIG. 27 shows saturation binding FACS results with Ab1 target-expressing HEK-293F cells for Ab1, Vel-IPV-Ab1, and Vel-IPV-Ab1 Coil 10 (described in Table 2).

FIG. 28 shows saturation binding ELISA results for hB6H12.3, Vel-IPV-hB6H12.3, and Vel variant Coil 10 (described in Table 2) against recombinant human CD47. Vel-IPV-anti-CD47=hB6H12.3 masked with wildtype Vel.

FIG. 29 shows saturation binding ELISA for trastuzumab, Vel-IPV-trastuzumab, and Vel variant Coil 10 (described in Table 2) against recombinant human HER2. Vel-IPV-trastuzumab=trastuzumab masked with wildtype Vel.

FIG. 30 shows saturation binding ELISA results for rituximab, Vel-IPV-rituximab, and Vel variant Coil 10 (described in Table 2) against recombinant human CD20. Vel-IPV-rituximab=rituximab masked with wildtype Vel.

FIG. 31 shows saturation binding ELISA results for Ab1, Vel-IPV-Ab1, and several Vel-IPV-Ab1 variants (Coils 103-151) against recombinant integrin αvβ6 antigen. Variants are as described in Table 15.

FIG. 32 shows stability of Vel-IPV-Ab1 and several Vel-IPV-Ab1 variants in vivo, as assessed by Western blot analysis of cleaved heavy chain at 24 or 48 hours post-intravenous injection in nude mice. Variants are as described in Table 17.

FIG. 33 shows saturation binding ELISA for trastuzumab, Vel-IPV-trastuzumab, and several Vel variants against recombinant human HER2. Variants are described in Table 20. Vel-IPV-trastuzumab=trastuzumab masked with wildtype Vel.

FIG. 34 shows saturation binding ELISA results for hB6H12.3, Vel-IPV-hB6H12.3, and several additional Vel variants against recombinant human CD47. Vel-IPV-hB6H12.3=hB6H12.3 masked with wildtype Vel. Variants are as described in Table 21.

FIG. 35 shows stability of Vel-IPV-trastuzumab and several Vel variants in vivo, as assessed by Western blot analysis of cleaved heavy chain at 24 or 48 hours post-intravenous injection in nude mice. Vel-IPV-trastuzumab=trastuzumab masked with wildtype Vel. Variants are as described in Table 20.

FIG. 36 shows stability of Vel-IPV-hB6H12.3 and several Vel variants in vivo, as assessed by Western blot analysis of cleaved heavy chain at 24 or 48 hours post-intravenous injection in nude mice. Vel-IPV-hB6H12.3=hB6H12.3 masked with wildtype Vel. Variants are as described in Table 21.

FIG. 37 shows saturation binding ELISA results for hB6H12.3, Vel-IPV-hB6H12.3, and several additional Vel variants against recombinant human CD47. Vel-IPV-hB6H12.3=hB6H12.3 masked with wildtype Vel. Variants are as described in Table 24.

FIG. 38 shows saturation binding ELISA results for hB6H12.3, Vel-IPV-hB6H12.3, and several additional Vel variants against recombinant human CD47. Vel-IPV-hB6H12.3=hB6H12.3 masked with wildtype Vel. Variants are as described in Table 25.

FIG. 39 shows saturation binding flow cytometry analysis against L540cy cells that express CD47 antigen for hB6H12.3, Vel-IPV-hB6H12.3, and several additional Vel variants. Vel-IPV-hB6H12.3=hB6H12.3 masked with wildtype Vel. Variants are as described in Table 25.

FIG. 40 shows saturation binding flow cytometry analysis against Ramos cells that express human CD47 antigen for hB6H12.3, Vel-IPV-hB6H12.3, and Vel variants. Vel-IPV-hB6H12.3=hB6H12.3 masked with wildtype Vel. Variants are as described in Table 26.

FIG. 41 shows hemoglobin (HGB) levels following a single dose of anti-CD37 IgG1 antibody (1 mg/kg) in cynomolgus macaques. Vel-IPV-hB6H12.3=hB6H12.3 masked with wildtype Vel. Coil 163 is as described in Table 26.

FIG. 42 shows circular dichroism studies to determine the relative heterodimeric affinities of the Vel (light chain of SEQ ID NO: 186, heavy chain of SEQ ID NO: 187), Coil 10 (light chain of SEQ ID NO: 188, heavy chain of SEQ ID NO: 189), and Coil 148 (light chain of SEQ ID NO: 190, heavy chain of SEQ ID NO: 191) peptides.

FIG. 43 shows circular dichroism experiments with VelA (light chain) peptide, Coil 10 (L24A), and Coil 148 (L24A/V28L) VelA mutants.

FIG. 44 shows circular dichroism experiments with VelB (heavy chain) peptide, Coil 10 (L24I) and Coil 148 (L24I/V28L) VelB mutants.

### DETAILED DESCRIPTION

The invention provides improved masking domains that may be used in masked antibodies, wherein the masked antibodies comprising the improved masking domains demonstrate reduced aggregation. In the masked antibody, the variable regions are masked by linkage of the variable region chains to coiled-coil forming polypeptides. The coiled-coil forming polypeptides associate with one another to form coiled coils (i.e., the respective peptides each form coils and these coils are coiled around each other) and, in some embodiments, sterically inhibit binding of the antibody binding site to its target. These coiled-coil polypeptides may be linked to the heavy chain and light chain variable regions of the antibody. Masking of antibodies by this format can reduce binding affinities (and cytotoxic activities in the case of ADCs) by over one hundred-fold or by over a thousand-fold, and in some embodiments, can reduce off-target effects. In some instances, however, masked antibodies may aggregate in solution, which may be undesirable in a pharmaceutical formulation. In some embodiments, the improved masking domains described herein reduce the aggregation of masked antibodies.

In certain exemplary embodiments, antibodies are provided that comprise a removable mask (e.g., a mask comprising a coiled coil domain) that blocks binding of the antibody to its antigenic target. In certain embodiments, an improved masking coiled-coil domain is attached to the amino-terminus of one or more of the heavy and/or light chains of the antibody via a matrix metalloproteinase (MMP)-cleavable linker sequence. In a tumor microenvironment, for example, altered proteolysis leads to unregulated tumor growth, tissue remodeling, inflammation, tissue invasion, and metastasis. See, e.g., Kessenbrock (2011) Cell 141:52 MMPs represent the most prominent family of proteinases associated with tumorigenesis, and MMPs mediate many of the changes in the microenvironment during tumor progression. Id. Upon exposure of the antibody of the present invention to an MMP, the MMP linker sequence is cleaved, thus allowing removal of the coiled coil mask and enabling the antibody to bind its target antigen in a tumor microenvironment-specific manner.

In other embodiments, such as for use in vitro, such as in medical diagnostics, chemical processing, or industrial uses, masked antibodies may be useful so that antibody activity can be controlled by addition of an exogenous protease to the solution at an appropriate point to cleave off the coiled-coils of the mask and allow the antibodies to bind to their targets. Regardless of the application, however, addition of coiled-coil masks to antibodies could increase the risk of aggregation when the antibodies are stored in concentrated form. The improved masking domains described herein may address this concern by reducing aggregation of solutions comprising the antibodies.

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

Compositions or methods “comprising” one or more recited elements or steps may include other elements or steps not specifically recited. For example, a composition that comprises antibody may contain the antibody alone or in combination with other ingredients.

Compositions or methods “consisting essentially of” one or more steps may include elements or steps not specifically recited so long as any additional element or step does not materially alter the essential nature of the composition or method as recited in the claim. For example, other steps may be included so long as they do not materially alter the overall preparation process, such as wash steps or buffer changes.

Unless otherwise apparent from the context, when a value is expressed as “about” X or “approximately” X, the stated value of X will be understood to be accurate to ±10%.

Solvates in the context of the invention are those forms of the compounds of the invention that form a complex in the solid or liquid state through coordination with solvent molecules. Hydrates are one specific form of solvates, in which the coordination takes place with water. In certain exemplary embodiments, solvates in the context of the present invention are hydrates.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present invention, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

The term “antibody” denotes immunoglobulin proteins produced by the body in response to the presence of an antigen and that bind to the antigen, as well as antigen-binding fragments and engineered variants thereof. Hence, the term “antibody” includes, for example, intact monoclonal antibodies (e.g., antibodies produced using hybridoma technology) and it also encompasses antigen-binding antibody fragments, such as a F(ab′)₂, a Fv fragment, a diabody, a single-chain antibody, an scFv fragment, or an scFv-Fc. Genetically engineered intact antibodies and fragments such as chimeric antibodies, humanized antibodies, single-chain Fv fragments, single-chain antibodies, diabodies, minibodies, linear antibodies, bispecific or bivalent, multivalent or multi-specific (e.g., bispecific) hybrid antibodies, and the like. Thus, the term “antibody” is used expansively to include any protein that comprises an antigen-binding site of an antibody and is capable of specifically binding to its antigen. In some embodiments, an antibody comprises two amino-termini (for example, comprises two polypeptide chains), such as a heavy chain (or fragment thereof) amino-terminus and a light chain (or fragment thereof) amino-terminus.

The term “antibody” includes a “naked” antibody that is not bound (i.e., covalently or non-covalently bound) to a masking compound of the invention. The term antibody also embraces a “masked” antibody, which comprises an antibody that is covalently or non-covalently bound to one or more masking compounds such as, e.g., coiled coil peptides, as described further herein. The term antibody includes a “conjugated” antibody or an “antibody-drug conjugate (ADC)” in which an antibody is covalently or non-covalently bound to a pharmaceutical agent, e.g., to a cytostatic or cytotoxic drug. In certain embodiments, an antibody is a naked antibody or antigen-binding fragment that optionally is conjugated to a pharmaceutical agent, e.g., to a cytostatic or cytotoxic drug. In other embodiments, an antibody is a masked antibody or antigen-binding fragment that optionally is conjugated to a pharmaceutical agent, e.g., to a cytostatic or cytotoxic drug.

Antibodies typically comprise a heavy chain variable region and a light chain variable region, each comprising three complementary determining regions (CDRs) with surrounding framework (FR) regions, for a total of six CDRs. An antibody light or heavy chain variable region (also referred to herein as a “light chain variable domain” (“VL domain”) or “heavy chain variable domain” (“VH domain”), respectively) comprises “framework” regions interrupted by three “complementarity determining regions” or “CDRs.” The framework regions serve to align the CDRs for specific binding to an epitope of an antigen. Thus, the term “CDR” refers to the amino acid residues of an antibody that are primarily responsible for antigen binding. From amino-terminus to carboxyl-terminus, both VL and VH domains comprise the following framework (FR) and CDR regions: FRI, CDR1, FR2, CDR2, FR3, CDR3, FR4.

Naturally occurring antibodies are usually tetrameric and consist of two identical pairs of heavy and light chains. In each pair, the light and heavy chain variable regions (VL and VH) are together primarily responsible for binding to an antigen, and the constant regions are primarily responsible for the antibody effector functions. Five classes of antibodies (IgG, IgA, IgM, IgD, and IgE) have been identified in higher vertebrates. IgG comprises the major class, and it normally exists as the second most abundant protein found in plasma. In humans, IgG consists of four subclasses, designated IgG1, IgG2, IgG3, and IgG4. Each immunoglobulin heavy chain possesses a constant region that comprises constant region protein domains (CH1, hinge, CH2, and CH3; IgG3 also contains a CH4 domain) that are substantially invariant for a given subclass in a species. Antibodies as defined herein, may include these natural forms as well as various antigen-binding fragments, as described above, antibodies with modified heavy chain constant regions, bispecific and multispecific antibodies, and masked antibodies.

The assignment of amino acids to each variable region domain is in accordance with the definitions of Kabat, Sequences of Proteins of Immunological Interest (National Institutes of Health, Bethesda, MD, 1987 and 1991). Kabat also provides a widely used numbering convention (Kabat numbering) in which corresponding residues between different heavy chain variable regions or between different light chain variable regions are assigned the same number. CDRs 1, 2 and 3 of a VL domain are also referred to herein, respectively, as CDR-L1, CDR-L2 and CDR-L3. CDRs 1, 2 and 3 of a VH domain are also referred to herein, respectively, as CDR-H1, CDR-H2 and CDR-H3. If so noted, the assignment of CDRs can be in accordance with IMGT® (Lefranc et al., Developmental & Comparative Immunology 27:55-77; 2003) in lieu of Kabat.

An “antigen-binding site” of an antibody is that portion of an antibody that is sufficient to bind to its antigen. The minimum such region is typically a fragment of a variable domain comprising six CDRs (or three CDRs in the case of a single-domain antibody). In some embodiments, an antigen-binding site of an antibody comprises both a heavy chain variable (VH) domain and a light chain variable (VL) domain that bind to a common epitope. Within the context of the present invention, an antibody may include one or more components in addition to an antigen-binding site, such as, for example, a second antigen-binding site of an antibody (which may bind to the same or a different epitope or to the same or a different antigen), a peptide linker, an immunoglobulin constant region, an immunoglobulin hinge, an amphipathic helix (see Pack and Pluckthun, Biochem. 31:1579-1584, 1992), a non-peptide linker, an oligonucleotide (see Chaudri et al, FEBS Letters 450:23-26, 1999), a cytostatic or cytotoxic drug, and the like, and may be a monomeric or multimeric protein. Examples of molecules comprising an antigen-binding site of an antibody are known in the art and include, for example, Fv, single-chain Fv (scFv), Fab, Fab′, F(ab′)2, F(ab) c, diabodies, minibodies, nanobodies, Fab-scFv fusions, bispecific (scFv)4-IgG, and bispecific (scFv)2-Fab. (See, e.g., Hu et al, Cancer Res. 56:3055-3061, 1996; Atwell et al., Molecular Immunology 33:1301-1312, 1996; Carter and Merchant, Curr. Op. Biotechnol. 8:449-454, 1997; Zuo et al., Protein Engineering 13:361-367, 2000; and Lu et al., J. Immunol. Methods 267:213-226, 2002.)

Numbering of the heavy chain constant region is via the EU index as set forth in Kabat (Kabat, Sequences of Proteins of Immunological Interest, National Institutes of Health, Bethesda, MD, 1987 and 1991).

Unless the context dictates otherwise, the term “monoclonal antibody” is not limited to antibodies produced through hybridoma technology. The term “monoclonal antibody” can include an antibody that is derived from a single clone, including any eukaryotic, prokaryotic or phage clone. In particular embodiments, the antibodies described herein are monoclonal antibodies.

The term “chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

The term “humanized VH domain” or “humanized VL domain” refers to an immunoglobulin VH or VL domain comprising some or all CDRs entirely or substantially from a non-human donor immunoglobulin (e.g., a mouse or rat) and variable domain framework sequences entirely or substantially from human immunoglobulin sequences. The non-human immunoglobulin providing the CDRs is called the “donor” and the human immunoglobulin providing the framework is called the “acceptor.” In some instances, humanized antibodies will retain some non-human residues within the human variable domain framework regions to enhance proper binding characteristics (e.g., mutations in the frameworks may be required to preserve binding affinity when an antibody is humanized).

A “humanized antibody” is an antibody comprising one or both of a humanized VH domain and a humanized VL domain. Immunoglobulin constant region(s) need not be present, but if they are, they are entirely or substantially from human immunoglobulin constant regions.

Although humanized antibodies often incorporate all six CDRs (preferably as defined by Kabat or IMGT®) from a mouse antibody, they can also be made with fewer than all six CDRs (e.g., at least 3, 4, or 5) from a mouse antibody (e.g., Pascalis et al., J. Immunol. 169:3076, 2002; Vajdos et al., Journal of Molecular Biology, 320:415-428, 2002; Iwahashi et al., Mol. Immunol. 36:1079-1091, 1999; Tamura et al, Journal of Immunology, 164:1432-1441, 2000).

A CDR in a humanized antibody is “substantially from” a corresponding CDR in a non-human antibody when at least 60%, at least 85%, at least 90%, at least 95% or 100% of corresponding residues (as defined by Kabat (or IMGT)) are identical between the respective CDRs. In particular variations of a humanized VH or VL domain in which CDRs are substantially from a non-human immunoglobulin, the CDRs of the humanized VH or VL domain have no more than six (e.g., no more than five, no more than four, no more than three, no more than two, or nor more than one) amino acid substitutions (preferably conservative substitutions) across all three CDRs relative to the corresponding non-human VH or VL CDRs. The variable region framework sequences of an antibody VH or VL domain or, if present, a sequence of an immunoglobulin constant region, are “substantially from” a human VH or VL framework sequence or human constant region, respectively, when at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% of corresponding residues (as defined by Kabat numbering for the variable region and EU numbering for the constant region), or about 100% of corresponding residues (as defined by Kabat numbering for the variable region and EU numbering for the constant region) are identical. Hence, all parts of a humanized antibody, except the CDRs, are typically entirely or substantially from corresponding parts of natural human immunoglobulin sequences.

Two amino acid sequences have “100% amino acid sequence identity” if the amino acid residues of the two amino acid sequences are the same when aligned for maximal correspondence. Sequence comparisons can be performed using standard software programs such as those included in the LASERGENE bioinformatics computing suite, which is produced by DNASTAR (Madison, Wisconsin). Other methods for comparing two nucleotide or amino acid sequences by determining optimal alignment are well-known to those of skill in the art. (See, e.g., Peruski and Peruski, The Internet and the New Biology: Tools for Genomic and Molecular Research (ASM Press, Inc. 1997); Wu et al. (eds.), “Information Superhighway and Computer Databases of Nucleic Acids and Proteins,” in Methods in Gene Biotechnology 123-151 (CRC Press, Inc. 1997); Bishop (ed.), Guide to Human Genome Computing (2nd ed., Academic Press, Inc. 1998).) Two amino acid sequences are considered to have “substantial sequence identity” if the two sequences have at least about 80%, at least about 85%, at about least 90%, or at least about 95% sequence identity relative to each other.

Percentage sequence identities are determined with antibody sequences maximally aligned by the Kabat numbering convention. After alignment, if a subject antibody region (e.g., the entire variable domain of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage.

Specific binding of an antibody to its target antigen typically refers an affinity of at least about 10⁶, about 10⁷, about 10⁸, about 10⁹, or about 10¹⁰ M⁻¹. Specific binding is detectably higher in magnitude and distinguishable from non-specific binding occurring to at least one non-specific target. Specific binding can be the result of formation of bonds between particular functional groups or particular spatial fit (e.g., lock and key type), whereas nonspecific binding is typically the result of van der Waals forces.

The term “epitope” refers to a site of an antigen to which an antibody binds. An epitope can be formed from contiguous amino acids or noncontiguous amino acids juxtaposed by tertiary folding of one or more proteins. Epitopes formed from contiguous amino acids are typically retained upon exposure to denaturing agents, e.g., solvents, whereas epitopes formed by tertiary folding are typically lost upon treatment with denaturing agents, e.g., solvents. An epitope typically includes at least about 3, and more usually, at least about 5, at least about 6, at least about 7, or about 8-10 amino acids in a unique spatial conformation. Methods of determining spatial conformation of epitopes include, for example, x-ray crystallography and two-dimensional nuclear magnetic resonance. See, e.g., Epitope Mapping Protocols, in Methods in Molecular Biology, Vol. 66, Glenn E. Morris, Ed. (1996).

Antibodies that recognize the same or overlapping epitopes can be identified in a simple immunoassay showing the ability of one antibody to compete with the binding of another antibody to a target antigen. The epitope of an antibody can also be defined by X-ray crystallography of the antibody bound to its antigen to identify contact residues.

Alternatively, two antibodies have the same epitope if all amino acid mutations in the antigen that reduce or eliminate binding of one antibody reduce or eliminate binding of the other (provided that such mutations do not produce a global alteration in antigen structure). Two antibodies have overlapping epitopes if some amino acid mutations that reduce or eliminate binding of one antibody reduce or eliminate binding of the other antibody.

Competition between antibodies can be determined by an assay in which a test antibody inhibits specific binding of a reference antibody to a common antigen (see, e.g., Junghans et al., Cancer Res. 50:1495, 1990). A test antibody competes with a reference antibody if an excess of a test antibody inhibits binding of the reference antibody.

Antibodies identified by competition assay (competing antibodies) include antibodies that bind to the same epitope as the reference antibody and antibodies that bind to an adjacent epitope sufficiently proximal to the epitope bound by the reference antibody for steric hindrance to occur. Antibodies identified by a competition assay also include those that indirectly compete with a reference antibody by causing a conformational change in the target protein thereby preventing binding of the reference antibody to a different epitope than that bound by the test antibody.

An antibody effector function refers to a function contributed by an Fc region of an Ig. Such functions can be, for example, antibody-dependent cellular cytotoxicity (ADCC), antibody-dependent cellular phagocytosis (ADCP), or complement-dependent cytotoxicity (CDC). Such function can be affected by, for example, binding of an Fc region to an Fc receptor on an immune cell with phagocytic or lytic activity or by binding of an Fc region to components of the complement system. Typically, the effect(s) mediated by the Fc-binding cells or complement components result in inhibition and/or depletion of the targeted cell. Fc regions of antibodies can recruit Fc receptor (FcR)-expressing cells and juxtapose them with antibody-coated target cells. Cells expressing surface FcR for IgGs including FcγRIII (CD16), FcγRII (CD32) and FcγRIII (CD64) can act as effector cells for the destruction of IgG-coated cells. Such effector cells include monocytes, macrophages, natural killer (NK) cells, neutrophils and eosinophils. Engagement of FcγR by IgG activates ADCC or ADCP. ADCC is mediated by CD16+ effector cells through the secretion of membrane pore-forming proteins and proteases, while phagocytosis is mediated by CD32+ and CD64+ effector cells (see Fundamental Immunology, 4^{th} ed., Paul ed., Lippincott-Raven, N.Y., 1997, Chapters 3, 17 and 30; Uchida et al., J. Exp. Med. 199: 1659-69, 2004; Akewanlop et al., Cancer Res. 61: 4061-65, 2001; Watanabe et al., Breast Cancer Res. Treat. 53:199-207, 1999).

In addition to ADCC and ADCP, Fc regions of cell-bound antibodies can also activate the complement classical pathway to elicit CDC. C1q of the complement system binds to the Fc regions of antibodies when they are complexed with antigens. Binding of C1q to cell-bound antibodies can initiate a cascade of events involving the proteolytic activation of C4 and C2 to generate the C3 convertase. Cleavage of C3 to C3b by C3 convertase enables the activation of terminal complement components including C5b, C6, C7, C8 and C9. Collectively, these proteins form membrane-attack complex pores on the antibody-coated cells. These pores disrupt the cell membrane integrity, killing the target cell (see Immunobiology, 6th ed., Janeway et al, Garland Science, N. Y., 2005, Chapter 2).

The term “antibody-dependent cellular cytotoxicity” or “ADCC” refers to a mechanism for inducing cell death that depends on the interaction of antibody-coated target cells with immune cells possessing lytic activity (also referred to as effector cells). Such effector cells include natural killer cells, monocytes/macrophages and neutrophils. The effector cells attach to an Fc region of Ig bound to target cells via their antigen-combining sites. Death of the antibody-coated target cell occurs as a result of effector cell activity.

The term “antibody-dependent cellular phagocytosis” or “ADCP” refers to the process by which antibody-coated cells are internalized, either in whole or in part, by phagocytic immune cells (e.g., by macrophages, neutrophils and/or dendritic cells) that bind to an Fc region of Ig.

The term “complement-dependent cytotoxicity” or “CDC” refers to a mechanism for inducing cell death in which an Fc region of a target-bound antibody activates a series of enzymatic reactions culminating in the formation of holes in the target cell membrane.

Typically, antigen-antibody complexes such as those on antibody-coated target cells bind and activate complement component C1q, which in turn activates the complement cascade leading to target cell death. Activation of complement may also result in deposition of complement components on the target cell surface that facilitate ADCC by binding complement receptors (e.g., CR3) on leukocytes.

A “therapeutic antigen” refers to an antigen that may be targeted by an antibody to achieve a beneficial therapeutic effect. In some embodiments, an antibody that binds to a therapeutic antigen may agonize, i.e., increase the activity of, the antigen. In some embodiments, an antibody that binds to a therapeutic antigen may antagonize, i.e., decrease the activity of, the antigen. In some embodiments, an antibody may bind a therapeutic antigen and achieve a beneficial effect by bringing another molecule or cell to the antigen (or to the cell that expresses the antigen). Nonlimiting examples of antibodies bringing another molecule or cell to the therapeutic antigen include, for example, an antibody-drug conjugate that brings a cytotoxic drug to a cell that expresses the therapeutic antigen; a bispecific antibody that brings a cytotoxic cell to the cell the expresses the therapeutic antigen (such as a bispecific antibody comprising an anti-CD3 binding domain, which recruits a cytotoxic T cell. For the avoidance of doubt, both antigens bound by a therapeutic bispecific antibody are considered therapeutic antigens.

An “antibody-drug conjugate” refers to an antibody conjugated to a cytotoxic agent or cytostatic agent. Typically, antibody-drug conjugates bind to a target antigen on a cell surface, followed by internalization of the antibody-drug conjugate into the cell and subsequent release of the drug into the cell.

Typically, antigen-antibody complexes such as those on antibody-coated target cells bind and activate complement component C1q, which in turn activates the complement cascade leading to target cell death. Activation of complement may also result in deposition of complement components on the target cell surface that facilitate ADCC by binding complement receptors (e.g., CR3) on leukocytes.

A “cytotoxic effect” refers to the depletion, elimination and/or killing of a target cell. A “cytotoxic agent” refers to a compound that has a cytotoxic effect on a cell, thereby mediating depletion, elimination and/or killing of a target cell. In certain embodiments, a cytotoxic agent is conjugated to an antibody or administered in combination with an antibody. Suitable cytotoxic agents are described further herein.

A “cytostatic effect” refers to the inhibition of cell proliferation. A “cytostatic agent” refers to a compound that has a cytostatic effect on a cell, thereby mediating inhibition of growth and/or expansion of a specific cell type and/or subset of cells. Suitable cytostatic agents are described further herein.

The terms “patient” and “subject” refer to organisms to be treated by the methods described herein and includes human and other mammalian subjects such as non-human primates, mammals (e.g., murines, simians, equines, bovines, porcines, canines, felines, and the like), rabbits, rats, mice, and the like and transgenic species thereof, that receive either prophylactic or therapeutic treatment. In certain exemplary embodiments, a subject is a human patient suffering from or at risk of developing cancer, e.g., a solid tumor, that optionally secretes one or more proteases capable of cleaving a masking domain (e.g., a coiled coil masking domain) of an antibody described herein.

As used herein, the terms, “treat,” “treatment” and “treating” includes any effect, e.g., lessening, reducing, modulating, ameliorating or eliminating, that results in the improvement of the condition, disease, disorder, and the like, or ameliorating a symptom thereof, such as for example, reduced number of cancer cells, reduced tumor size, reduced rate of cancer cell infiltration into peripheral organs, or reduced rate of tumor metastasis or tumor growth.

As used herein, the term “effective amount” refers to the amount of a compound (e.g., a masked antibody) sufficient to effect beneficial or desired results. An effective amount of an antibody is administered in an “effective regimen.” The term “effective regimen” refers to a combination of amount of the antibody being administered and dosage frequency adequate to accomplish prophylactic or therapeutic treatment of the disorder.

The term “pharmaceutically acceptable” means approved or approvable by a regulatory agency of the Federal or a state government or listed in the U.S. Pharmacopeia or other generally recognized pharmacopeia for use in animals, and more particularly in humans. The term “pharmaceutically compatible ingredient” refers to a pharmaceutically acceptable diluent, adjuvant, excipient, or vehicle with which an antibody is formulated.

The phrase “pharmaceutically acceptable salt,” refers to pharmaceutically acceptable organic or inorganic salts. Exemplary salts include sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1, l′-methylene bis-(2 hydroxy-3-naphthoate) salts. A pharmaceutically acceptable salt may further comprise an additional molecule such as, e.g., an acetate ion, a succinate ion or other counterion. A counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion.

### I. Masking Domains Comprising Coiled-Coils

In certain embodiments, an antibody is associated with a masking domain comprising coiled coil domains (also referred to as a “coiled coil masking domain”) that blocks binding of the antibody to its antigen target. In various embodiments, an antibody associated with a masking domain is referred to as a “masked antibody.”

A coiled coil is a structural motif in proteins and peptides in which two or more alpha-helices wind around each other to form a supercoil. There can be two, three or four helices in a coiled coil bundle and the helices can either run in the same (parallel) or in the opposite (antiparallel) directions.

Coiled coils typically comprise sequence elements of three and four residues whose hydrophobicity pattern and residue composition are compatible with the structure of amphipathic alpha-helices. The alternating three and four residue sequence elements constitute heptad repeats in which the amino acids are designated ‘a,’ ‘b,’ ‘c,’ ‘d,’ ‘e,’ ‘f’ and ‘g.’ Residues in positions ‘a’ and ‘d’ are generally hydrophobic and form a zig-zag pattern of knobs and holes that interlock with a similar pattern on another strand to form a tight-fitting hydrophobic core. Of the remaining residues, ‘b,’ ‘c’ and ‘f’ tend to be charged. Therefore, the formation of a heptad repeat depends on the physical properties of hydrophobicity and charge that are required at a particular position, not on a specific amino acid. In certain exemplary embodiments, coiled coils of the present invention are formed from two coiled coil-forming peptides.

In some embodiments, a masking domain is provided that comprises a first coiled-coil domain and a second coiled-coil domain, wherein the first coiled-coil domain and/or the second coiled-coil domain comprises at least one amino acid substitution that reduces aggregation of a masked antibody comprising the masking domain in an aqueous formulation, compared to the masked antibody without the at least one amino acid substitution in the same aqueous formulation. In some embodiments, the at least one amino acid substitution reduces aggregation of the masked antibody in an aqueous formulation at pH6-8.5 compared to the masked antibody without the at least one amino acid substitution in the same aqueous formulation. In some embodiments, the at least one amino acid substitution reduces homodimerization of the first coiled coil domain and/or the second coiled coil domain. That is, in various embodiments, the first and second coiled-coil domains are different, and at least one substitution reduces homodimerization of one or both of the coiled-coil domains. In some embodiments, the at least one amino acid substitution increases heterodimerization of the first and second coiled-coil domains. By reducing homodimerization and/or increasing heterodimerization, a masked antibody comprising the first and second coiled-coil domains, in some embodiments, will have reduced aggregation. In some embodiments, at least one amino acid substitution reduces affinity of the first coiled-coil domain for the second coiled-coil domain without substantially increasing homodimerization.

In some embodiments, a first coiled coil domain (i.e., a coiled coil-forming peptide) comprises the sequence V₇D₈E₉L₁₀Q₁₁A₁₂E₁₃V₁₄D₁₅Q₁₆L₁₇E₁₈D₁₉E₂₀N₂₁Y₂₂A₂₃L₂₄K₂₅T₂₆K₂₇V₂₈A₂₉Q₃₀L₃₁R₃₂K₃₃K₃₄V₃₅ E₃₆K₃₇L₃₈ (SEQ ID NO: 2), and a second coiled coil domain comprises the sequence V₇A₈Q₉L₁₀E₁₁E₁₂K₁₃V₁₄K₁₅T₁₆L₁₇R₁₈A₁₉E₂₀N₂₁Y₂₂E₂₃L₂₄K₂₅S₂₆E₂₇V₂₈Q₂₉R₃₀L₃₁E₃₂E₃₃Q₃₄V₃₅ A₃₆Q₃₇L₃₈ (SEQ ID NO: 1); wherein the first coiled coil domain and/or the second coiled-coil domain comprises at least one amino acid substitution in SEQ ID NO: 2 or SEQ ID NO: 1. In some embodiments, the at least one amino acid substitution reduces aggregation of a masked antibody comprising the first and second coiled coil domains in an aqueous formulation compared to the same masked antibody without the at least one amino acid substitution (i.e., compared to the same masked antibody comprising masking domains comprising SEQ ID NOs: 1 and 2 linked through the same linker to the same antibody chains). In some embodiments, the at least one amino acid substitution reduces aggregation of a masked antibody comprising the first and second coiled coil domains in an aqueous formulation at pH6-8 compared to the same masked antibody without the at least one amino acid substitution (i.e., compared to the same masked antibody comprising masking domains comprising SEQ ID NOs: 1 and 2 linked through the same linker to the same antibody chains).

In some embodiments, the amino acid substitution reduces aggregation of the masked antibody in an aqueous formulation at pH 6.5, 6.6, 6.7, 6.8, 6.9, 7, 7.1, 7.2, 7.3, 7.4, 7.5, 7.6, 7.7, and/or 7.8.

In some embodiments, the aqueous formulation comprises 10-100 mM potassium phosphate and/or sodium phosphate, 10-100 mM NaCl and/or KCl. In some embodiments, the formulation comprises 1-10 mM EDTA. In some embodiments, the aqueous formulation comprises 80 mM potassium phosphate and/or sodium phosphate, 50 mM NaCl and/or KCl, and 5 mM EDTA, adjusted to the desired pH. In some embodiments, the aqueous formulation is PBS.

In some embodiments, the at least one amino acid substitution reduces aggregation of a masked antibody comprising the first and second coiled coil domains in an aqueous formulation comprising salt compared to the same masked antibody without the at least one amino acid substitution (i.e., in some embodiments, compared to the same masked antibody comprising masking domains comprising SEQ ID NOs: 1 and 2 linked through the same linker to the same antibody chains). In some embodiments, the amino acid substitution reduces aggregation of the masked antibody in an aqueous formulation comprising 30 mM, 40 mM, 50 mM, 60 mM, 70 mM, 80 mM, 90 mM, 100 mM, 110 mM, 120 mM, 130 mM, 140 mM, or 150 mM salt. In some embodiments, the salt comprises NaCl, KCl, and/or MgCl₂.

In various embodiments, at least one amino acid substitution in the first coiled coil domain and/or the second coiled coil domain replaces an acidic amino acid with a non-acidic amino acid. Replacement of one or more acidic amino acids, in some instances, reduces aggregation of a masked antibody comprising the first and second coiled coil domains. In some such embodiments, the acidic amino acid that is replaced is aspartic acid or glutamic acid, and the non-acidic amino acid may be any amino acid other than aspartic acid or glutamic acid. In various embodiments, each non-acid amino acid is independently selected from asparagine, glutamine, lysine, histidine, arginine, serine, phenylalanine, tyrosine, tryptophan, threonine, leucine, isoleucine, and methionine. In various embodiments, each non-acid amino acid is independently selected from asparagine, glutamine, lysine, histidine, arginine, and serine. Any number of acidic amino acids may be replaced, including one, two, three, or four acidic amino acids in the first coiled-coil domain and/or one, two, three, or four acidic amino acids in the second coiled-coil domain.

In some embodiments, at least one acidic amino acid that is replaced in the first coiled coil domain is selected from D8, E9, E13, D15, D19, and E36 of SEQ ID NO: 2. In various embodiments, at least one acidic amino acid that is replaced in the second coiled coil domain is selected from E11, E12, E20, E23, E32, and E33 of SEQ ID NO: 1. In some embodiments, the first coiled-coil domain comprises amino acid substitutions at D8 and/or E36, such as D8K and/or E36H of SEQ ID NO: 2.

In various embodiments, the masked antibody comprises a first coiled-coil domain comprising a first substitution of a first hydrophobic amino acid with a less bulky hydrophobic amino acid or a more bulky hydrophobic amino acid, and a second coiled-coil domain comprising a second substitution of a second hydrophobic amino acid with a less bulky hydrophobic amino acid or a more bulky hydrophobic amino acid. Such substitutions may be combined with the acidic amino acid substitutions discussed herein. In some such embodiments, one coiled-coil domain comprises the less bulky hydrophobic amino acid substitution and one coiled-coil domain comprises the more bulky hydrophobic amino acid substitution. In some embodiments, the first hydrophobic amino acid and the second hydrophobic amino acid are at the same amino acid position in the coiled coil domains (i.e., are at the same amino acid position when aligned with SEQ ID NOs: 2 and 1, respectively). Without intending to be bound by any particular theory, by replacing one hydrophobic amino acid in one coiled coil domain with a less bulky amino acid and replacing one hydrophobic amino acid at the corresponding position in the other coiled coil domain with a more bulky amino acid, in some instances, heterodimerization may be favored over homodimerization.

In some embodiments, the first hydrophobic amino acid replaced (on the first coiled coil domain) and the second hydrophobic amino acid replaced (on the second coiled coil domain) are both valine or leucine. In some embodiments, the less bulky hydrophobic amino acid is alanine, glycine, or serine. In some embodiments, the more bulky hydrophobic amino acid is isoleucine, phenylalanine, tyrosine, tryptophan, or methionine.

In some embodiments, the first coiled-coil and the second coiled-coil comprise a substitution at position 24 and/or at position 28.

In some embodiments, one, two, three, or four pairs of hydrophobic amino acids (a pair being one hydrophobic amino acid at the same position on each coiled coil domain) are substituted. Nonlimiting exemplary pairs of hydrophobic amino acids that may be substituted in coiled-coil domains comprising SEQ ID NO: 2 and SEQ ID NO: 1 include V14/V14, L17/L17, L24/L24, V28/V28, L31/L31, and V35/V35, wherein the first position is the position in the first coiled-coil domain and the second position is the position in the second coiled-coil domain. Nonlimiting exemplary pairs of hydrophobic amino acid substitutions in coiled-coil domains comprising SEQ ID NOs: 2 and 1 include V14A/V14I, V14I/V14A, L17A/L17I, L17I/L17A, L24A/L241, L24I/L24A, L24G/L24Y, L24Y/L24G, L24A/L24Y, L24Y/L24A, L24Y/L24W, L24W/L24Y, L24Y/L24F, L24F/L24Y, L24S/L24F, L24F/L24S, L24Y/L24Y, L24S/L24S, L24G/L24W, L24W/L24G, L24G/L24F, L24F/L24G, L24A/L24W, L24W/L24A, L24W/L24F, L24F/L24W, L24A/L24F, L24F/L24A, L24W/L24S, L24S/L24W, L24Y/L24S, L24S/L24Y, L24F/L24F, L24I/L24V, L24V/L24I, L24K/L24I, L24I/L24K, V28A/V28I, V28I/V28A, V28N/V28N, V28L/V28I, V28I/V28L, V28K/V28I, V28I/V28K, V28L/V28L, L31A/L31I, L31I/L31A, V35A/V35I, V35I/V35A, and V35N/V35N. In some embodiments, a pair of hydrophobic amino acid substitutions is L24A/L24I, L24I/L24A, L24V/L24A, L24A/L24V, V28A/V28I, V28I/V28A, V28L/V28L, L31A/L31I, or L31I/L31A. In some embodiments, a pair of hydrophobic substitutions is L24A/L24I, L24I/L24A, L24V/L24A, L24A/L24V, V28A/V28I, V28I/V28A, V28L/V28L.

In some embodiments, the coiled-coil domains may comprise two pairs of hydrophobic substitutions, including, for example, in coiled-coil domains comprising SEQ ID NOs: 2 and 1, wherein the first pair of substitutions is selected from L24A/L241 and L24I/L24A, and the second pair of substitutions is selected from L31A/L311 and L311/L31A; or wherein the first pair of substitutions is selected from L17A/L17I and L17I/L17A, and the second pair of substitutions is selected from L31A/L311 and L311/L31A; or wherein the first pair of substitutions is selected from L17A/L171 and L17I/L17A, and the second pair of substitutions is selected from L24A/L24I and L24I/L24A; or wherein the first pair of substitutions is selected from V28A/V281 and V28I/V28A, and the second pair of substitutions is selected from L31A/L311 and L31I/L31A.

In some embodiments, the coiled-coil domains may comprise three pairs of hydrophobic substitutions, including, for example, in coiled-coil domains comprising SEQ ID NOs: 2 and 1, wherein the first pair of substitutions is selected from L17A/L171 and L17I/L17A, the second pair of substitutions is selected from L24A/L241 and L24I/L24A, and the third pair of substitutions is selected from L31A/L311 and L31I/L31A.

Nonlimiting exemplary coiled-coil domains that may be used in masked antibodies comprising coiled-coil domains comprising SEQ ID NOs: 2 and 1 comprise:
a. a first coiled-coil domain comprising substitutions L24A and L31A, and a second coiled-coil domain comprising substitutions L24I and L31I; or
b. a first coiled-coil domain comprising substitutions L241 and L31I, and a second coiled-coil domain comprising substitutions L24A and L31A; or
c. a first coiled-coil domain comprising substitutions L17I and L31I, and a second coiled-coil domain comprising substitutions L17A and L31A; or
d. a first coiled-coil domain comprising substitutions L171, L24I, and L31I, and a second coiled-coil domain comprising substitutions L17A, L24A, and L31A; or
e. a first coiled-coil domain comprising substitutions V281 and L31I, and a second coiled-coil domain comprising substitutions V28A and L31A; or
f. a first coiled-coil domain comprising substitution L31I, and a second coiled-coil domain comprising substitutions L31A and E33K; or
g. a first coiled-coil domain comprising substitutions D8K and E36H, and a second coiled-coil domain comprising no substitutions; or
h. a first coiled-coil domain comprising substitutions D8K, E36H, and L31I, and a second coiled-coil domain comprising substitution L31A; or
i. a first coiled-coil domain comprising substitutions D8K, D15K, and L31I, and a second coiled-coil domain comprising substitution L31A; or
j. a first coiled-coil domain comprising substitutions D15K, E36H, and L31I, and a second coiled-coil domain comprising substitution L31A.

Nonlimiting exemplary coiled-coil domains that may be used in masked antibodies comprising coiled-coil domains comprising SEQ ID NOs: 2 and 1 comprise:
a. a first coiled-coil domain comprising a substitution L24V, and a second coiled-coil domain comprising a substitution L24A; or
b. a first coiled-coil domain comprising a substitution L24A, and a second coiled-coil domain comprising a substitution L24V; or
c. a first coiled-coil domain comprising substitutions L24V and V281, and a second coiled-coil domain comprising a substitution L24A; or
d. a first coiled-coil domain comprising a substitution L24A, and a second coiled-coil domain comprising substitutions L24V and V28I; or
e. a first coiled-coil domain comprising substitutions L241 and V28L, and a second coiled-coil domain comprising substitutions L24A and V28L; or
f. a first coiled-coil domain comprising substitutions L24A and V28L, and a second coiled-coil domain comprising substitutions L241 and V28L; or
g. a first coiled-coil domain comprising substitutions L24V and V28I, and a second coiled-coil domain comprising substitutions T6S and L24A; or
h. a first coiled-coil domain comprising substitutions T6S and L24A, and a second coiled-coil domain comprising substitutions L24V and V28I; or
i. a first coiled-coil domain comprising substitutions L241 and V28L, and a second coiled-coil domain comprising substitutions T6S, L24A and V28L; or
j. a first coiled-coil domain comprising substitutions T6S, L24A and V28L, and a second coiled-coil domain comprising substitutions L241 and V28L.

Nonlimiting exemplary coiled-coil domains that may be used in masked antibodies comprise an amino acid sequence selected from SEQ ID NOs: 1, 2, 5-121, and 142-185. In some embodiments, a first coiled-coil domain comprises an amino acid sequence selected from SEQ ID NOs: 2, 59-121, 141, and 169-185. In some embodiments, a second coiled-coil domain comprises an amino acid sequence selected from SEQ ID NOs: 1, 5-58, and 142-168.

In some embodiments, the first and second coiled-coil domains comprise the sequences of SEQ ID NOs: 66 and 11, respectively; or SEQ ID NOs: 70 and 15, respectively; or SEQ ID NOs: 72 and 17, respectively; or SEQ ID NOs: 94 and 1, respectively; or SEQ ID NOs: 108 and 1, respectively; or SEQ ID NOs: 108 and 11, respectively; or SEQ ID NOs: 110 and 49, respectively; or SEQ ID NOs: 111 and 50, respectively; or SEQ ID NOs: 112 and 51, respectively; or SEQ ID NOs: 113 and 52, respectively; or SEQ ID NOs: 114 and 53, respectively; or SEQ ID NOs: 70 and 56, respectively; or SEQ ID NOs: 116 and 1, respectively; or SEQ ID NOs: 117 and 15, respectively; or SEQ ID NOs: 118 and 15, respectively; or SEQ ID NOs: 119 and 15, respectively; or SEQ ID NOs: 120 and 57, respectively; or SEQ ID NOs: 121 and 57, respectively; or SEQ ID NOs: 63 and 8, respectively.

In some embodiments, the first and second coiled-coil domains comprise the sequences of SEQ ID NOs: 181 and 168, respectively; or SEQ ID NOs: 180 and 167, respectively; or SEQ ID NOs: 181 and 155, respectively; or SEQ ID NOs: 180 and 11, respectively.

In any of the embodiments described herein, each masking domain of the masked antibody may comprise an amino-terminal sequence selected from SEQ ID NOs: 138 and 139. In some embodiments, the first masking domain comprises the amino-terminal sequence of SEQ ID NO: 139 and the second masking domain comprises the amino-terminal sequence of SEQ ID NO: 138. In some embodiments, each masking domain comprises an amino-terminal sequence of SEQ ID NO: 139.

In any of the embodiments described herein, each masking domain may comprise a protease-cleavable linker. In some such embodiments, the masking domain is linked to the heavy chain or light chain via the protease-cleavable linker.

Sequences shown for light chains may be used with heavy chains and vice versa. Thus, in some embodiments, what is referred to as the “first” masking domain may be linked to the heavy chain or the light chain, and the “second” masking domain may be linked to the other chain. Thus, in some embodiments, the first masking domain is linked to the amino-terminus of the heavy chain and the second masking domain is linked to the amino-terminus of the light chain, and in some embodiments, the first masking domain is linked to the amino-terminus of the light chain and the second masking domain is linked to the amino-terminus of the heavy chain. Optionally, multiple copies of the coiled coil domains are linked in tandem to the amino-termini of the heavy and light chains.

In some cases, antigen binding is reduced at least 100-fold by the presence of a masking domain (e.g., a coiled coil masking domain). In some embodiments, antigen binding is reduced by more than 200-fold or more than 1500-fold, by the presence of a masking domain (e.g., a coiled coil masking domain). In some embodiments, cytotoxicity of the conjugate is reduced at least 100-fold by the presence of a masking domain (e.g., a coiled coil masking domain). In some embodiments, cytotoxicity of the conjugate is reduced at least 200-fold, or at least 1500-fold by the presence of a masking domain (e.g., a coiled coil masking domain).

In some embodiments, additional amino acids other than those described herein are substituted in the coiled-coil domains. In some such embodiments, the additional substitution(s) do not significantly alter the properties of the coiled-coil domain comprising the substitution. In certain exemplary embodiments, additional amino acid substitutions are conservative substitutions. For purposes of classifying amino acid substitutions as conservative or nonconservative, the following amino acid substitutions are considered conservative substitutions: serine substituted by threonine, alanine, or asparagine; threonine substituted by proline or serine; asparagine substituted by aspartic acid, histidine, or serine; aspartic acid substituted by glutamic acid or asparagine; glutamic acid substituted by glutamine, lysine, or aspartic acid; glutamine substituted by arginine, lysine, or glutamic acid; histidine substituted by tyrosine or asparagine; arginine substituted by lysine or glutamine; methionine substituted by isoleucine, leucine or valine; isoleucine substituted by leucine, valine, or methionine; leucine substituted by valine, isoleucine, or methionine; phenylalanine substituted by tyrosine or tryptophan; tyrosine substituted by tryptophan, histidine, or phenylalanine; proline substituted by threonine; alanine substituted by serine; lysine substituted by glutamic acid, glutamine, or arginine; valine substituted by methionine, isoleucine, or leucine; and tryptophan substituted by phenylalanine or tyrosine.

### Linkers and Cleavage Sites

In certain embodiments of the invention, a masking domain comprises a linker, which is located between the coiled-coil domain and the antibody chain to which the coiled-coil domain is attached. The linkers can be any segments of amino acids conventionally used as linkers for joining peptide domains. Suitable linkers can vary in length, such as from 1-20, 2-15, 3-12, 4-10, 5, 6, 7, 8, 9 or 10 amino acid. Some such linkers include a segment of polyglycine. Some such linkers include one or more serine residues, often at positions flanking the glycine residues. Other linkers include one or more alanine residues. Glycine and glycine-serine polymers are relatively unstructured, and therefore may be able to serve as a neutral tether between components. Glycine accesses significantly more phi-psi space than even alanine, and is much less restricted than residues with longer side chains (see Scheraga, Rev. Computational Chem. 11173-142 (1992)). Some exemplary linkers are in the form S(G)nS, wherein n is from 5-20. Other exemplary linkers are (G) n, glycine-serine polymers (including, for example, (GS) n, (GSGGS)n [(GSGGS) is SEQ ID NO: 126) and (GGGS)n, [(GGGS) is SEQ ID NO: 127) where n is an integer of at least one), glycine-alanine polymers, alanine-serine polymers, and other flexible linkers known in the art. Some examples of linkers are Ser-(Gly)10-Ser (SEQ ID NO: 128), Gly-Gly-Ala-Ala (SEQ ID NO: 129), Gly-Gly-Gly-Gly-Ser (SEQ ID NO: 130), Leu-Ala-Ala-Ala-Ala (SEQ ID NO: 131), Gly-Gly-Ser-Gly (SEQ ID NO: 132), Gly-Gly-Ser-Gly-Gly (SEQ ID NO: 133), Gly-Ser-Gly-Ser-Gly (SEQ ID NO: 134), Gly-Ser-Gly-Gly-Gly (SEQ ID NO: 135), Gly-Gly-Gly-Ser-Gly (SEQ ID NO: 136), Gly-Ser-Ser-Ser-Gly (SEQ ID NO: 137), and the like.

The protease site is preferably recognized and cleaved by a protease expressed extracellularly so it contacts a masked antibody, releasing the masked antibody and allowing it to contact its target, such as a receptor extracellular domain or soluble ligand. Several matrix metalloproteinase sites (MMP1-28) are suitable. MMPs play a role in tissue remodeling and are implicated in neoplastic processes such as morphogenesis, angiogenesis and metastasis. Some exemplary protease sites are PLG-XXX, a well-known endogenous sequence for MMPs, PLG-VR (WO2014193973; SEQ ID NO: 125) and IPVSLRSG (SEQ ID NO: 122) (Turk et al., Nat. Biotechnol., 2001, 19, 661-667), LSGRSDNY (SEQ ID NO: 124) (Cytomx) and GPLGVR (SEQ ID NO: 122) (Chang et al., Clin. Cancer Res. 2012 Jan. 1; 18 (1): 238-47). Additional examples of MMPs are provided in US 2013/0309230, WO 2009/025846, WO 2010/081173, WO 2014/107599, WO 2015/048329, US20160160263, and Ratnikov et al., Proc. Natl. Acad. Sci. USA, 111: E4148-E4155 (2014).

TABLE 1
Protease cleavage sequences. The MMP-cleavage
site is indicated by * while the uPA/
matriptase/legumain cleavage sites
are indicated by **.

| Cleavage | |
|---|---|
| Site Name | Sequence |
| M2 | GPLG*VR** (SEQ ID NQ: 123) |
| IPV | IPVS*LR**SG (SEQ ID NQ: 122) |

In various embodiments, a masking domain comprises a coiled-coil domain, a linker, and a protease cleavage sequence. In some such embodiments, the linker and the protease cleavage site comprise the sequence GSIPVSLRSG (SEQ ID NO: 140). In various embodiments, a masked antibody comprises two different masking domains (i.e., comprising different coiled coil domains provided herein), each of which comprises the linker-protease site sequence GSIPVSLRSG (SEQ ID NO: 140).

### II. Pharmaceutical Compositions

For therapeutic use, a masked antibody is preferably combined with a pharmaceutically acceptable carrier. As used herein, “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the compositions and not deleterious to the recipient. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

Accordingly, masked antibodies of the present invention can comprise at least one of any suitable excipients, such as, but not limited to, diluent, binder, stabilizer, buffers, salts, lipophilic solvents, preservative, adjuvant or the like. Pharmaceutically acceptable excipients are preferred. Non-limiting examples of, and methods of preparing such sterile solutions are well known in the art, such as, but not limited to, those described in Gennaro, Ed., Remington's Pharmaceutical Sciences, 18th Edition, Mack Publishing Co. (Easton, Pa.) 1990. Pharmaceutically acceptable carriers can be routinely selected that are suitable for the mode of administration, solubility and/or stability of the antibody molecule, fragment or variant composition as well known in the art or as described herein.

In some embodiments, compositions of masked antibodies are aqueous compositions. In other embodiments, the compositions are lyophilized.

Pharmaceutical compositions of a masked antibody as disclosed herein can be presented in a dosage unit form, or can be stored in a form suitable for supplying more than one unit dose. A pharmaceutical composition should be formulated to be compatible with its intended route of administration. Lyophilized formulations are typically reconstituted in solution prior to administration or use, whereas aqueous formulations may be “ready to use,” meaning that they are administered directly, without being first diluted for example, or can be diluted in saline or another solution prior to use.

Examples of routes of administration are intravenous (IV), intradermal, intratumoral, inhalation, transdermal, topical, transmucosal, and rectal administration. The phrases “parenteral administration” and “administered parenterally” as used herein means modes of administration other than enteral and topical administration, usually by injection, and includes, without limitation, intravenous, intramuscular, subcutaneous, intraarterial, intrathecal, intracapsular, intraorbital, intravitreous, intracardiac, intradermal, intraperitoneal, transtracheal, inhaled, subcutaneous, subcuticular, intraarticular, subcapsular, subarachnoid, intraspinal, epidural and intrasternal injection and infusion.

Pharmaceutical compositions are preferably sterile. Sterilization can be accomplished by any suitable method, e.g., filtration through sterile filtration membranes. Where the composition is lyophilized, filter sterilization can be conducted prior to or following lyophilization and reconstitution.

In some embodiments, an aqueous formulation at pH 6-8 comprising a masked antibody with an improved masking domain provided herein exhibits reduced aggregation after at least 1 day, at least 2 days, at least 3 days, or at least 1 week at 25° C. compared to a masked antibody comprising the same antibody and a VelA/VelB masking domain in the same formulation at the same temperature after the same time period. In some embodiments, an aqueous reconstitution at pH 6-8 of a lyophilized formulation comprising a masked antibody with an improved masking domain provided herein exhibits reduced aggregation after at least 1 day, at least 2 days, at least 3 days, or at least 1 week at 25° C. compared to a masked antibody comprising the same antibody and a VelA/VelB masking domain in the same formulation at the same temperature after the same time period.

The present invention also provides a kit, comprising packaging material and at least one vial comprising a composition of masked antibody as described herein. The kit may further comprise instructions for use and/or a diluent solution if the antibody formulation must be diluted prior to use. The present invention also provides a kit, comprising packaging material and at least one vial comprising a lyophilized composition of masked antibody as described herein. The kit may further comprise instructions for use, a reconstitution solution for reconstituting the antibody into solution, and/or a diluent solution if the antibody composition is to be further diluted after reconstitution.

### III. Exemplary Antibodies

Antibodies include non-human, humanized, human, chimeric, and veneered antibodies, nanobodies, dAbs, scFV's, Fabs, and the like. Some such antibodies are immunospecific for a cancer cell antigen, preferably one on the cell surface internalizable within a cell on antibody binding. In some embodiments, the antibody portion of a masked antibody binds a therapeutic antigen. Such therapeutic antigens include antigens that may be targeted for treatment of any disease or disorder, including, but not limited to, cancer, autoimmune disorders, and infections.

Targets to which antibodies can be directed include receptors on cancer cells and their ligands or counter-receptors (i.e., tumor-associated antigens). Such targets include, but are not limited to, CD3, CD19, CD20, CD22, CD30, CD33, CD34, CD40, CD44, CD47, CD52, CD70, CD79a, CD123, Her-2, EphA2, lymphocyte associated antigen 1, VEGF or VEGFR, CTLA-4, LIV-1, nectin-4, CD74, SLTRK-6, EGFR, CD73, PD-L1, CD163, CCR4, CD147, EpCam, Trop-2, CD25, C5aR, Ly6D, alpha v integrin, B7H3, B7H4, Her-3, folate receptor alpha, GD-2, CEACAM5, CEACAM6, c-MET, CD266, MUC1, CD10, MSLN, sialyl Tn, Lewis Y, CD63, CD81, CD98, CD166, tissue factor (CD142), CD55, CD59, CD46, CD164, TGF beta receptor 1 (TGFBR1), TGFBR2, TGFBR3, FasL, MerTk, Axl, Clec 12A, CD352, FAP, CXCR3, and CD5.

In some embodiments, a masked antibody provided herein may be useful for treating an autoimmune disease. Nonlimiting antigens that may be bound by an antibody useful for treating an autoimmune disease include TNF-α, IL-1, IL-2R, IL-6, IL-12, IL-23, IL-17, IL-17R, BLyS, CD20, CD52, α4β7 integrin, and α4-integrin.

Some examples of commercial antibodies and their targets suitable for use in the masked antibodies described herein include, but are not limited to, brentuximab or brentuximab vedotin, CD30, alemtuzumab, CD52, rituximab, CD20, trastuzumab Her/neu, nimotuzumab, cetuximab, EGFR, bevacizumab, VEGF, palivizumab, RSV, abciximab, GpIIb/IIIa, infliximab, adalimumab, certolizumab, golimumab TNF-alpha, baciliximab, daclizumab, IL-2R, omalizumab, IgE, gemtuzumab or vadastuximab, CD33, natalizumab, VLA-4, vedolizumab alpha4beta7, belimumab, BAFF, otelixizumab, teplizumab CD3, ofatumumab, ocrelizumab CD20, epratuzumab CD22, alemtuzumumab CD52, eculizumab C5, canakimumab IL-1beta, mepolizumab IL-5, reslizumab, tocilizumab IL-6R, ustekinumab, briakinumab IL-12, 23, hBU12 (CD19) (US20120294853), humanized 1F6 or 2F12 (CD70) (US20120294863), BR2-14a and BR2-22a (LIV-1) (WO2012078688).

### Glycosylation Variants

Antibodies may be glycosylated at conserved positions in their constant regions (Jefferis and Lund, (1997) Chem. Immunol. 65:111-128; Wright and Morrison, (1997) TibTECH 15:26-32). The oligosaccharide side chains of the immunoglobulins affect the protein's function (Boyd et al., (1996) Mol. Immunol. 32:1311-1318; Wittwe and Howard, (1990) Biochem. 29:4175-4180), and the intramolecular interaction between portions of the glycoprotein which can affect the conformation and presented three-dimensional surface of the glycoprotein (Jefferis and Lund, supra; Wyss and Wagner, (1996) Current Op. Biotech. 7:409-416). Oligosaccharides may also serve to target a given glycoprotein to certain molecules based upon specific recognition structures. For example, it has been reported that in agalactosylated IgG, the oligosaccharide moiety ‘flips’ out of the inter-CH2 space and terminal N-acetylglucosamine residues become available to bind mannose binding protein (Malhotra et al., (1995) Nature Med. 1:237-243). Removal by glycopeptidase of the oligosaccharides from CAMPATH-1H (a recombinant humanized murine monoclonal IgG1 antibody which recognizes the CDw52 antigen of human lymphocytes) produced in Chinese Hamster Ovary (CHO) cells resulted in a complete reduction in complement mediated lysis (CMCL) (Boyd et al., (1996) Mol. Immunol. 32:1311-1318), while selective removal of sialic acid residues using neuraminidase resulted in no loss of DMCL. Glycosylation of antibodies has also been reported to affect antibody-dependent cellular cytotoxicity (ADCC). In particular, CHO cells with tetracycline-regulated expression of α(1,4)-N-acetylglucosaminyltransferase III (GnTIII), a glycosyltransferase catalyzing formation of bisecting GlcNAc, was reported to have improved ADCC activity (Umana et al. (1999) Mature Biotech. 17:176-180).

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Glycosylation variants of antibodies are variants in which the glycosylation pattern of an antibody is altered. By altering is meant deleting one or more carbohydrate moieties found in the antibody, adding one or more carbohydrate moieties to the antibody, changing the composition of glycosylation (glycosylation pattern), the extent of glycosylation, etc.

Addition of glycosylation sites to an antibody can be accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites). Similarly, removal of glycosylation sites can be accomplished by amino acid alteration within the native glycosylation sites of the antibody.

The amino acid sequence is usually altered by altering the underlying nucleic acid sequence. These methods include isolation from a natural source (in the case of naturally-occurring amino acid sequence variants) or preparation by oligonucleotide-mediated (or site-directed) mutagenesis, PCR mutagenesis, and cassette mutagenesis of an earlier prepared variant or a non-variant version of the antibody.

The glycosylation (including glycosylation pattern) of antibodies may also be altered without altering the amino acid sequence or the underlying nucleotide sequence. Glycosylation largely depends on the host cell used to express the antibody. Since the cell type used for expression of recombinant glycoproteins, e.g., antibodies, as potential therapeutics is rarely the native cell, significant variations in the glycosylation pattern of the antibodies can be expected. See, e.g., Hse et al., (1997) J. Biol. Chem. 272:9062-9070. In addition to the choice of host cells, factors which affect glycosylation during recombinant production of antibodies include growth mode, media formulation, culture density, oxygenation, pH, purification schemes and the like. Various methods have been proposed to alter the glycosylation pattern achieved in a particular host organism including introducing or overexpressing certain enzymes involved in oligosaccharide production (U.S. Pat. Nos. 5,047,335; 5,510,261; 5,278,299). Glycosylation, or certain types of glycosylation, can be enzymatically removed from the glycoprotein, for example using endoglycosidase H (Endo H). In addition, the recombinant host cell can be genetically engineered, e.g., make defective in processing certain types of polysaccharides. These and similar techniques are well known in the art.

The glycosylation structure of antibodies can be readily analyzed by conventional techniques of carbohydrate analysis, including lectin chromatography, NMR, Mass spectrometry, HPLC, GPC, monosaccharide compositional analysis, sequential enzymatic digestion, and HPAEC-PAD, which uses high pH anion exchange chromatography to separate oligosaccharides based on charge. Methods for releasing oligosaccharides for analytical purposes are also known, and include, without limitation, enzymatic treatment (commonly performed using peptide-N-glycosidase F/endo-β-galactosidase), elimination using harsh alkaline environment to release mainly O-linked structures, and chemical methods using anhydrous hydrazine to release both N- and O-linked oligosaccharides.

A preferred form of modification of glycosylation of antibodies is reduced core fucosylation. “Core fucosylation” refers to addition of fucose (“fucosylation”) to N-acetylglucosamine (“GlcNAc”) at the reducing terminal of an N-linked glycan.

A “complex N-glycoside-linked sugar chain” is typically bound to asparagine 297 (according to the number of Kabat). As used herein, the complex N-glycoside-linked sugar chain has a biantennary composite sugar chain, mainly having the following structure:

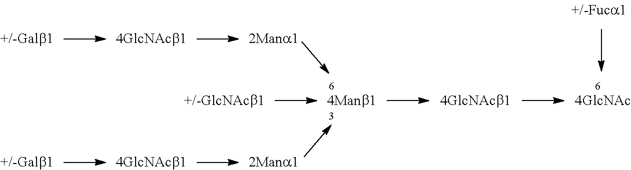

where +/−indicates the sugar molecule can be present or absent, and the numbers indicate the position of linkages between the sugar molecules. In the above structure, the sugar chain terminal which binds to asparagine is called a reducing terminal (at right), and the opposite side is called a non-reducing terminal. Fucose is usually bound to N-acetylglucosamine (“GlcNAc”) of the reducing terminal, typically by an α1,6 bond (the 6-position of GlcNAc is linked to the 1-position of fucose). “Gal” refers to galactose, and “Man” refers to mannose.

A “complex N-glycoside-linked sugar chain” includes 1) a complex type, in which the non-reducing terminal side of the core structure has one or more branches of galactose-N-acetylglucosamine (also referred to as “gal-GlcNAc”) and the non-reducing terminal side of Gal-GlcNAc optionally has a sialic acid, bisecting N-acetylglucosamine or the like; or 2) a hybrid type, in which the non-reducing terminal side of the core structure has both branches of a high mannose N-glycoside-linked sugar chain and complex N-glycoside-linked sugar chain.

In some embodiments, the “complex N-glycoside-linked sugar chain” includes a complex type in which the non-reducing terminal side of the core structure has zero, one or more branches of galactose-N-acetylglucosamine (also referred to as “gal-GlcNAc”) and the non-reducing terminal side of Gal-GlcNAc optionally further has a structure such as a sialic acid, bisecting N-acetylglucosamine or the like.

According to certain methods, only a minor amount of fucose is incorporated into the complex N-glycoside-linked sugar chain(s) of an antibody. For example, in various embodiments, less than about 60%, less than about 50%, less than about 40%, less than about 30%, less than about 20%, less than about 15%, less than about 10%, less than about 5%, or less than about 3% of the molecules of an antibody have core fucosylation by fucose. In some embodiments, about 2% of the molecules of the antibody has core fucosylation by fucose.

In certain embodiments, only a minor amount of a fucose analog (or a metabolite or product of the fucose analog) is incorporated into the complex N-glycoside-linked sugar chain(s). For example, in various embodiments, less than about 60%, less than about 50%, less than about 40%, less than about 30%, less than about 20%, less than about 15%, less than about 10%, less than about 5%, or less than about 3% of the antibodies have core fucosylation by a fucose analog or a metabolite or product of the fucose analog. In some embodiments, about 2% of the antibodies have core fucosylation by a fucose analog or a metabolite or product of the fucose analog.

Methods of making non-fucosylated antibodies (which may be used to make non-fucosylated masked antibodies) by incubating antibody-producing cells with a fucose analogue are described, e.g., in WO2009/135181. Briefly, cells that have been engineered to express the antibody are incubated in the presence of a fucose analogue or an intracellular metabolite or product of the fucose analog. An intracellular metabolite can be, for example, a GDP-modified analog or a fully or partially de-esterified analog. A product can be, for example, a fully or partially de-esterified analog. In some embodiments, a fucose analogue can inhibit an enzyme(s) in the fucose salvage pathway. For example, a fucose analog (or an intracellular metabolite or product of the fucose analog) can inhibit the activity of fucokinase, or GDP-fucose-pyrophosphorylase. In some embodiments, a fucose analog (or an intracellular metabolite or product of the fucose analog) inhibits fucosyltransferase (preferably a 1,6-fucosyltransferase, e.g., the FUT8 protein). In some embodiments, a fucose analog (or an intracellular metabolite or product of the fucose analog) can inhibit the activity of an enzyme in the de novo synthetic pathway for fucose. For example, a fucose analog (or an intracellular metabolite or product of the fucose analog) can inhibit the activity of GDP-mannose 4,6-dehydratase or/or GDP-fucose synthetase. In some embodiments, the fucose analog (or an intracellular metabolite or product of the fucose analog) can inhibit a fucose transporter (e.g., GDP-fucose transporter).

In one embodiment, the fucose analogue is 2-flurofucose. Methods of using fucose analogues in growth medium and other fucose analogues are disclosed, e.g., in WO/2009/135181, which is herein incorporated by reference.

Other methods for engineering cell lines to reduce core fucosylation included gene knock-outs, gene knock-ins and RNA interference (RNAi). In gene knock-outs, the gene encoding FUT8 (alpha 1,6-fucosyltransferase enzyme) is inactivated. FUT8 catalyzes the transfer of a fucosyl residue from GDP-fucose to position 6 of Asn-linked (N-linked) GlcNac of an N-glycan. FUT8 is reported to be the only enzyme responsible for adding fucose to the N-linked biantennary carbohydrate at Asn297. Gene knock-ins add genes encoding enzymes such as GNTIII or a Golgi alpha mannosidase II. An increase in the levels of such enzymes in cells diverts monoclonal antibodies from the fucosylation pathway (leading to decreased core fucosylation), and having increased amount of bisecting N-acetylglucosamines. RNAi typically also targets FUT8 gene expression, leading to decreased mRNA transcript levels or knocking out gene expression entirely. Any of these methods can be used to generate a cell line that would be able to produce a non-fucosylated antibody.

Many methods are available to determine the amount of fucosylation on an antibody. Methods include, e.g., LC-MS via PLRP-S chromatography and electrospray ionization quadrupole TOF MS.

### IV. Linking Coiled Coil Masking Agents to Antibodies

Coiled coil forming peptides are linked to the amino-termini of antibody variable regions via a linker including a protease site. A typical antibody includes a heavy and light chain variable region, in which case a coiled coil forming peptide is linked to the amino-termini of each. A bivalent antibody has two binding sites, which may or may not be the same. In a normal monospecific antibody, the binding sites are the same and the antibody has two identical light and heavy chain pairs. In this case, each heavy chain is linked to the same coiled coil forming peptide and each light chain to the same coiled coil forming peptide (which may or may not be the same as the peptide linked to the heavy chain). In a bispecific antibody, the binding sites are different and formed from two different heavy and light chain pairs. In such a case, the heavy and light chain variable region of one binding site are respectively linked to coiled coil forming peptides as are the heavy and light chain variable regions of the other binding site. Typically both heavy chain variable regions are linked to the same type of coiled coil forming peptide as are both light chain variable regions.

A coiled coil-forming peptide can be linked to an antibody variable region via a linker including a protease site. Typically, the same linker with the same protease cleavage site is used for linking each heavy or light chain variable region of an antibody to a coiled coil peptide. The protease cleavage site should be one amenable to cleavage by a protease present extracellularly in the intended target tissue or pathology, such as a cancer, such that cleavage of the linker releases the antibody from the coiled coil masking its activity allowing the antibody to bind to its intended target, such as a cell-surface antigen or soluble ligand.

As well as the variable regions, a masked antibody typically includes all or part of a constant region, which can include any or all of a light chain constant region, CHI, hinge, CH2 and CH3 regions. As with other antibodies one or more carboxy-terminal residues can be proteolytically processed or derivatized.

Coiled coils can be formed from the same peptide forming a homodimer or two different peptides forming a heterodimer. For formation of a homodimer, light and heavy antibody chains are linked to the same coiled coil forming peptide. For formation of a heterodimer, light and heavy antibody chains are linked to different coiled coils peptides. For some pairs of coiled coil forming peptides, it is preferred that one of the pair be linked to the heavy chain and the other to the light chain of an antibody although the reverse orientation is also possible.

Each antibody chain can be linked to a single coiled coil forming peptide or multiple such peptides in tandem (e.g., two, three, four or five copies of a peptide). If the latter, the peptides in tandem linkage are usually the same. Also if tandem linkage is employed, light and heavy chains are usually linked to the same number of peptides.

Linkage of antibody chains to coiled coil forming peptides can reduce the binding affinity of an antibody by at least about 10-fold, at least about 50-fold, at least about 100-fold, at least about 200-fold, at least about 500-fold, at least about 1000-fold or at least about 1500-fold relative to the same antibody without such linkage or after cleavage of such linkage. In some such antibodies, binding affinity is reduced between about 50-5000-fold, between about 50-1500-fold, between about 100-1500-fold, between about 200-1500-fold, between about 500-1500-fold, between about 500-5000-fold, between about 50-1000-fold, between about 100-1000-fold, between about 200-1000-fold, between about 500-1000-fold, between about 50-500-fold, or between about 100-500-fold. Effector functions of the antibody, such as ADCC, phagocytosis, and CDC or cytotoxicity as a result of linkage to a drug in an antibody drug conjugate can be reduced by the same factors or ranges. Upon proteolytic cleavage that serves to unmask an antibody or otherwise remove the mask from the antibody, the restored antibody typically has an affinity or effect function that is within a factor of 2, 1.5 or preferably unchanged within experimental error compared with an otherwise identical control antibody, which has never been masked.

### V. Antibody-Drug Conjugates

In certain embodiments, a masked antibody may comprise an antibody drug conjugates (ADCs, also referred to herein as an “immunoconjugate”). Particular ADCs may comprise cytotoxic agents (e.g., chemotherapeutic agents), prodrug converting enzymes, radioactive isotopes or compounds, or toxins (these moieties being collectively referred to as a therapeutic agent). For example, an ADC can be conjugated to a cytotoxic agent such as a chemotherapeutic agent, or a toxin (e.g., a cytostatic or cytocidal agent such as, for example, abrin, ricin A, pseudomonas exotoxin, or diphtheria toxin). Examples of useful classes of cytotoxic agents include, for example, DNA minor groove binders, DNA replication inhibitors, DNA alkylating agents, NAMPT inhibitors, and tubulin inhibitors (i.e., antitubulins). Exemplary cytotoxic agents include, for example, auristatins, camptothecins, calicheamicins, duocarmycins, etoposides, enediyine antibiotics, maytansinoids (e.g., DM1, DM2, DM3, DM4), taxanes, benzodiazepines (e.g., pyrrolo[1,4] benzodiazepines, indolinobenzodiazepines, and oxazolidinobenzodiazepines including pyrrolo[1,4] benzodiazepine dimers, indolinobenzodiazepine dimers, and oxazolidinobenzodiazepine dimers), lexitropsins, taxanes, combretastatins, cryptophysins, and vinca alkaloids. Nonlimiting exemplary cytotoxig agents include auristatin E, AFP, AEB, AEVB, MMAF, MMAE, paclitaxel, docetaxel, doxorubicin, morpholino-doxorubicin, cyanomorpholino-doxorubicin, melphalan, methotrexate, mitomycin C, a CC-1065 analogue, CBI, calicheamicin, maytansine, an analog of dolastatin 10, rhizoxin, or palytoxin, epothilone A, epothilone B, nocodazole, colchicine, colcimid, estramustine, cemadotin, discodermolide, eleutherobin, a tubulysin, a plocabulin, and maytansine.

An ADC can be conjugated to a pro-drug converting enzyme. The pro-drug converting enzyme can be recombinantly fused to the antibody or chemically conjugated thereto using known methods. Exemplary pro-drug converting enzymes are carboxypeptidase G2, beta-glucuronidase, penicillin-V-amidase, penicillin-G-amidase, β-lactamase, β-glucosidase, nitroreductase and carboxypeptidase A.

Techniques for conjugating therapeutic agents to proteins, and in particular to antibodies, are well-known. (See, e.g., Alley et al., Current Opinion in Chemical Biology 2010 14:1-9; Senter, Cancer J., 2008, 14 (3): 154-169.) The therapeutic agent can be conjugated in a manner that reduces its activity unless it is cleaved off the antibody (e.g., by hydrolysis, by proteolytic degradation, or by a cleaving agent). In some aspects, the therapeutic agent is attached to the antibody with a cleavable linker that is sensitive to cleavage in the intracellular environment of the antigen-expressing cancer cell but is not substantially sensitive to the extracellular environment, such that the conjugate is cleaved from the antibody when it is internalized by the antigen-expressing cancer cell (e.g., in the endosomal or, for example by virtue of pH sensitivity or protease sensitivity, in the lysosomal environment or in the caveolear environment). In some embodiments, the therapeutic agent can also be attached to the antibody with a non-cleavable linker.

In certain exemplary embodiments, an ADC can include a linker region between a cytotoxic or cytostatic agent and the antibody. As noted supra, typically, the linker can be cleavable under intracellular conditions, such that cleavage of the linker releases the therapeutic agent from the antibody in the intracellular environment (e.g., within a lysosome or endosome or caveolea). The linker can be, e.g., a peptidyl linker that is cleaved by an intracellular peptidase or protease enzyme, including a lysosomal or endosomal protease. Cleaving agents can include cathepsins B and D and plasmin (see, e.g., Dubowchik and Walker, Pharm. Therapeutics 83:67-123, 1999). Most typical are peptidyl linkers that are cleavable by enzymes that are present in antigen-expressing cells. For example, a peptidyl linker that is cleavable by the thiol-dependent protease cathepsin-B, which is highly expressed in cancerous tissue, can be used (e.g., a linker comprising a Phe-Leu or a Val-Cit peptide).

A cleavable linker can be pH-sensitive, i.e., sensitive to hydrolysis at certain pH values. Typically, the pH-sensitive linker is hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. (See, e.g., U.S. Pat. Nos. 5,122,368; 5,824,805; 5,622,929; Dubowchik and Walker, Pharm. Therapeutics 83:67-123, 1999; Neville et al, Biol. Chem. 264:14653-14661, 1989.) Such linkers are relatively stable under neutral pH conditions, such as those in the blood, but are unstable at below pH 5.5 or 5.0, the approximate pH of the lysosome.

Other linkers are cleavable under reducing conditions (e.g., a disulfide linker). Disulfide linkers include those that can be formed using SATA (N-succinimidyl-S-acetylthioacetate), SPDP (N-succinimidyl-3-(2-pyridyldithio) propionate), SPDB (N-succinimidyl-3-(2-pyridyldithio) butyrate) and SMPT (N-succinimidyl-oxycarbonyl-alpha-methyl-alpha-(2-pyridyl-dithio) toluene), SPDB and SMPT. (See, e.g., Thorpe et al., Cancer Res. 47:5924-5931, 1987; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935.)

The linker can also be a malonate linker (Johnson et al, Anticancer Res. 15:1387-93, 1995), a maleimidobenzoyl linker (Lau et al., Bioorg-Med-Chem. 3:1299-1304, 1995), or a 3′-N-amide analog (Lau et al., Bioorg-Med-Chem. 3:1305-12, 1995).

The linker also can be a non-cleavable linker, such as an maleimido-alkylene or maleimide-aryl linker that is directly attached to the therapeutic agent and released by proteolytic degradation of the antibody.

Typically, the linker is not substantially sensitive to the extracellular environment, meaning that no more than about 20%, typically no more than about 15%, more typically no more than about 10%, and even more typically no more than about 5%, no more than about 3%, or no more than about 1% of the linkers in a sample of the ADC is cleaved when the ADC is present in an extracellular environment (e.g., in plasma). Whether a linker is not substantially sensitive to the extracellular environment can be determined, for example, by incubating independently with plasma both (a) the ADC (the “ADC sample”) and (b) an equal molar amount of unconjugated antibody or therapeutic agent (the “control sample”) for a predetermined time period (e.g., 2, 4, 8, 16, or 24 hours) and then comparing the amount of unconjugated antibody or therapeutic agent present in the ADC sample with that present in control sample, as measured, for example, by high performance liquid chromatography.

The linker can also promote cellular internalization. The linker can promote cellular internalization when conjugated to the therapeutic agent (i.e., in the milieu of the linker-therapeutic agent moiety of the ADC or ADC derivate as described herein). Alternatively, the linker can promote cellular internalization when conjugated to both the therapeutic agent and the antibody (i.e., in the milieu of the ADC as described herein).

The antibody can be conjugated to the linker via a heteroatom of the antibody. These heteroatoms can be present on the antibody in its natural state or can be introduced into the antibody. In some aspects, the antibody will be conjugated to the linker via a nitrogen atom of a lysine residue. In other aspects, the antibody will be conjugated to the linker via a sulfur atom of a cysteine residue. Methods of conjugating linker and drug-linkers to antibodies are known in the art.

Exemplary antibody-drug conjugates include auristatin based antibody-drug conjugates meaning that the drug component is an auristatin drug. Auristatins bind tubulin, have been shown to interfere with microtubule dynamics and nuclear and cellular division, and have anticancer activity. Typically the auristatin based antibody-drug conjugate comprises a linker between the auristatin drug and the antibody. The linker can be, for example, a cleavable linker (e.g., a peptidyl linker) or a non-cleavable linker (e.g., linker released by degradation of the antibody). Auristatins include MMAF, and MMAE. The synthesis and structure of exemplary auristatins are described in U.S. Pat. Nos. 7,659,241, 7,498,298, 2009-0111756, 2009-0018086, and 7,968, 687 each of which is incorporated herein by reference in its entirety and for all purposes.

Other exemplary antibody-drug conjugates include maytansinoid antibody-drug conjugates meaning that the drug component is a maytansinoid drug, and benzodiazepine antibody drug conjugates meaning that the drug component is a benzodiazepine (e.g., pyrrolo[1,4] benzodiazepine dimers, indolinobenzodiazepine dimers, and oxazolidinobenzodiazepine dimers).

In certain embodiments, an antibody may be combined with an ADC with binding specificity to a different target. Exemplary ADCs that may be combined with a masked antibody include brentuximab vedotin (anti-CD30 ADC), enfortumab vedotin (anti-nectin-4 ADC), ladiratuzumab vedotin (anti-LIV-1 ADC), denintuzumab mafodotin (anti-CD19 ADC), glembatumumab vedotin (anti-GPNMB ADC), anti-TIM-1 ADC, polatuzumab vedotin (anti-CD79b ADC), anti-MUC16 ADC, depatuxizumab mafodotin, telisotuzumab vedotin, anti-PSMA ADC, anti-C4.4a ADC, anti-BCMA ADC, anti-AXL ADC, tisotuumab vedotin (anti-tissue factor ADC).

### VI. Masked Antibody Expression

Nucleic acids encoding masked antibodies can be expressed in a host cell that contains endogenous DNA encoding a masked antibody used in the present invention. Such methods are well known in the art, e.g., as described in U.S. Pat. Nos. 5,580,734, 5,641,670, 5,733,746, and 5,733,761. Also see, e.g., Sambrook, et al., supra, and Ausubel, et al., supra. Those of ordinary skill in the art are knowledgeable in the numerous expression systems available for expression of a nucleic acid encoding a protein of the present invention. Illustrative of cell cultures useful for the production of the antibodies, masked antibodies, specified portions or variants thereof, are mammalian cells. Mammalian cell systems often will be in the form of monolayers of cells although mammalian cell suspensions or bioreactors can also be used. A number of suitable host cell lines capable of expressing intact glycosylated proteins have been developed in the art, and include the COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, hep G2 cells, P3X63Ag8.653, SP2/0-Ag14, HeLa cells and the like, which are readily available from, for example, American Type Culture Collection, Manassas, VA. Yeast and bacterial host cells may also be used and are well known to those of skill in the art. Other cells useful for production of nucleic acids or proteins of the present invention are known and/or available, for instance, from the American Type Culture Collection Catalogue of Cell Lines and hybridomas or other known or commercial sources.

Expression vectors can include one or more of the following expression control sequences, such as, but not limited to an origin of replication; a promoter (e.g., late or early SV40 promoters, the CMV promoter (U.S. Pat. Nos. 5,168,062; 5,385,839), an HSV tk promoter, a pgk (phosphoglycerate kinase) promoter, an EF-1 alpha promoter (U.S. Pat. No. 5,266,491), at least one human immunoglobulin promoter; an enhancer, and/or processing information sites, such as ribosome binding sites, RNA splice sites, polyadenylation sites (e.g., an SV40 large T Ag poly A addition site), and transcriptional terminator sequences). See, e.g., Ausubel et al., supra; Sambrook, et al., supra.

Expression vectors optionally include at least one selectable marker. Such markers include, e.g., but are not limited to, methotrexate (MTX), dihydrofolate reductase (DHFR, U.S. Pat. Nos. 4,399,216; 4,634,665; 4,656, 134; 4,956,288; 5,149,636; 5,179,017), ampicillin, neomycin (G418), mycophenolic acid, or glutamine synthetase (GS, U.S. Pat. Nos. 5,122,464; 5,770,359; and 5,827,739), resistance for eukaryotic cell culture, and tetracycline or ampicillin resistance genes for culturing in E. coli and other bacteria or prokaryotes. Appropriate culture media and conditions for the above-described host cells are known in the art. Suitable vectors will be readily apparent to the skilled artisan. Introduction of a vector construct into a host cell can be effected by calcium phosphate transfection, DEAE-dextran mediated transfection, cationic lipid-mediated transfection, electroporation, transduction, infection or other known methods. Such methods are described in the art, such as Sambrook, supra; Ausubel, supra.

The nucleic acid insert should be operatively linked to an appropriate promoter. The expression constructs will further contain sites for transcription initiation, termination and, in the transcribed region, a ribosome binding site for translation. The coding portion of the mature transcripts expressed by the constructs will preferably include a translation initiating at the beginning and a termination codon (e.g., UAA, UGA or UAG) appropriately positioned at the end of the mRNA to be translated, with UAA and UAG preferred for mammalian or eukaryotic cell expression.

The nucleic acid insert is optionally in frame with a coiled coil sequence and/or an MMP cleavage sequence, e.g., at the amino-terminus of one or more heavy chain and/or light chain sequences. Alternatively, a coiled coil sequence and/or an MMP cleavage sequence can be post-translationally added to an antibody, e.g., via a disulfide bond or the like.

When eukaryotic host cells are employed, polyadenylation or transcription terminator sequences are typically incorporated into the vector. An example of a terminator sequence is the polyadenylation sequence from the bovine growth hormone gene. Sequences for accurate splicing of the transcript can also be included. An example of a splicing sequence is the VP1 intron from SV40 (Sprague, et al. (1983) J. Virol. 45:773-781). Additionally, gene sequences to control replication in the host cell can be incorporated into the vector, as known in the art.

### VII. Masked Antibody Isolation and Purification

Masked antibodies used in the present formulations can be recovered and purified from recombinant cell cultures by methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (HPLC) can also be employed for purification. See, e.g., Colligan, Current Protocols in Immunology, or Current Protocols in Protein Science, John Wiley & Sons, New York, N.Y., (1997-2001).

In some embodiments, antibodies or masked antibodies described herein can be expressed in a modified form. For instance, a region of additional amino acids, particularly charged amino acids, can be added to the amino-terminus of an antibody to improve stability and persistence in the host cell, during purification, or during subsequent handling and storage. Also, peptide moieties can be added to an antibody or masked antibody to facilitate purification. Such regions can be removed prior to final preparation of an antibody or masked antibody. Such methods are described in many standard laboratory manuals, such as Sambrook, supra; Ausubel, et al., ed., Current Protocols In Molecular Biology, John Wiley & Sons, Inc., NY, N.Y. (1987-2001).

Antibodies and masked antibodies described herein can include purified products, products of chemical synthetic procedures, and products produced by recombinant techniques from a eukaryotic host, including, for example, yeast, higher plant, insect and mammalian cells. Depending upon the host employed in a recombinant production procedure, the antibody or masked antibody of the present invention can be glycosylated or can be non-glycosylated, with glycosylated preferred. Such methods are described in many standard laboratory manuals, such as Sambrook, supra; Ausubel, supra, Colligan, Protein Science, supra.

### VIII. Therapeutic Applications

In some embodiments, masked antibodies herein may be used in methods of therapeutic treatment. Nonlimiting exemplary diseases and disorders that may be treated with the masked antibodies provided herein include cancer, autoimmune disorders, and infections. Generally, any disease or disorder that may be treated with a therapeutic antibody may be treated with a masked antibody provided herein. In some embodiments, a masked antibody results in reduced side-effects compared to the unmasked version of the antibody, for example, because the masked antibody does not bind its antigen until the mask has been removed. By selecting suitable cleavable linkers between the coiled-coil masking domains and the antibody chains, the masked antibody will remain masked until it reaches the vicinity of its target antigen, particularly its target antigen at the site of the disease or disorder. For example, in some instances, by selecting a cleavable linker that is cleaved by a protease that is present at higher concentration near a tumor, the masked antibody may have an improved safety profile because the antibody does not significantly bind its antigen until it reaches the tumor.

In some embodiments, methods of treating cancer are provided.

Positive therapeutic effects in cancer can be measured in a number of ways (See, W. A. Weber, J. Null. Med. 50: 1S-10S (2009); Eisenhauer et al., supra). In some embodiments, response to a masked antibody is assessed using RECIST 1.1 criteria. In some embodiments, the treatment achieved by a therapeutically effective amount is any of a partial response (PR), a complete response (CR), progression free survival (PFS), disease free survival (DFS), objective response (OR) or overall survival (OS). The dosage regimen of a therapy described herein that is effective to treat a primary or a secondary hepatic cancer patient may vary according to factors such as the disease state, age, and weight of the patient, and the ability of the therapy to elicit an anti-cancer response in the subject. While an embodiment of the treatment method, medicaments and uses of the present invention may not be effective in achieving a positive therapeutic effect in every subject, it should do so in a statistically significant number of subjects as determined by any statistical test known in the art such as the Student's t-test, the chi2-test, the U-test according to Mann and Whitney, the Kruskal-Wallis test (H-test), Jonckheere-Terpstra-test and the Wilcoxon-test.

“RECIST 1.1 Response Criteria” as used herein means the definitions set forth in Eisenhauer et al., E. A. et al., Eur. J Cancer 45:228-247 (2009) for target lesions or non-target lesions, as appropriate, based on the context in which response is being measured.

“Tumor” as it applies to a subject diagnosed with, or suspected of having, a primary or a secondary hepatic cancer, refers to a malignant or potentially malignant neoplasm or tissue mass of any size. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms). Nonlimiting exemplary sarcomas include soft tissue sarcoma and osteosarcoma.

“Tumor burden” also referred to as “tumor load,” refers to the total amount of tumor material distributed throughout the body. Tumor burden refers to the total number of cancer cells or the total size of tumor(s) throughout the body, including lymph nodes and bone narrow. Tumor burden can be determined by a variety of methods known in the art, such as, e.g., by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

Nonlimiting exemplary autoimmune diseases that may be treated with a masked antibody include Crohn's disease, ulcerative colitis, rheumatoid arthritis, psoriatic arthritis, ankylosing spondylitis, uveitis, juvenile idiopathic arthritis, multiple sclerosis, psoriasis (including plaque psoriasis), systemic lupus erythematosus, granulomatosis with polyangiitis, microscopic polyangiitis, systemic sclerosis, idiopathic thrombocytopenia purpura, graft-versus-host disease, and autoimmune cytopenias.

As used herein, the term “effective amount” refers to the amount of a compound (e.g., a masked antibody) sufficient to effect beneficial or desired results. An effective amount can be administered in one or more administrations, applications or dosages and is not intended to be limited to a particular formulation or administration route. Generally, a therapeutically effective amount of active component is in the range of 0.01 mg/kg to 100 mg/kg, 0.1 mg/kg to 100 mg/kg, 1 mg/kg to 100 mg/kg, 0.01 mg/kg to 10 mg/kg, 0.1 mg/kg to 10 mg/kg, 1 mg/kg to 10 mg/kg. The dosage administered can vary depending upon known factors, such as the pharmacodynamic characteristics of the particular agent, and its mode and route of administration; the age, health, and weight of the recipient; the type and extent of disease or indication to be treated, the nature and extent of symptoms, kind of concurrent treatment, frequency of treatment, and the effect desired. The initial dosage can be increased beyond the upper level in order to rapidly achieve the desired blood-level or tissue-level. Alternatively, the initial dosage can be smaller than the optimum, and the daily dosage may be progressively increased during the course of treatment. Human dosage can be optimized, e.g., in a conventional Phase I dose escalation study designed to run from 0.5 mg/kg to 20 mg/kg. Dosing frequency can vary, depending on factors such as route of administration, dosage amount, serum half-life of the antibody, and the disease being treated. Exemplary dosing frequencies are once per day, once per week and once every two weeks.

In certain exemplary embodiments, the present invention provides a method for treating cancer in a cell, tissue, organ, animal or patient. In particular embodiments, the present invention provides a method for treating a solid cancer in a human. Examples of cancers include, but are not limited to, solid tumors, soft tissue tumors, hematopoietic tumors that give rise to solid tumors, and metastatic lesions. Examples of hematopoietic tumors that have the potential to give rise to solid tumors include, but are not limited to, diffuse large B-cell lymphomas (DLBCL), follicular lymphoma, myelodysplastic syndrome (MDS), a lymphoma, Hodgkin's disease, a malignant lymphoma, non-Hodgkin's lymphoma, Burkitt's lymphoma, multiple myeloma, Richter's Syndrome (Richter's Transformation) and the like. Examples of solid tumors include, but are not limited to, malignancies, e.g., sarcomas (including soft tissue sarcoma and osteosarcoma), adenocarcinomas, and carcinomas, of the various organ systems, such as those affecting head and neck (including pharynx), thyroid, lung (small cell or non-small cell lung carcinoma (NSCLC)), breast, lymphoid, gastrointestinal tract (e.g., oral, esophageal, stomach, liver, pancreas, small intestine, colon and rectum, anal canal), genitals and genitourinary tract (e.g., renal, urothelial, bladder, ovarian, uterine, cervical, endometrial, prostate, testicular), central nervous system (e.g., neural or glial cells, e.g., neuroblastoma or glioma), skin (e.g., melanoma) and the like. In certain embodiments, the solid tumor is an NMDA receptor positive teratoma. In other embodiments, the cancer is selected from breast cancer, colon cancer, pancreatic cancer (e.g., a pancreatic neuroendocrine tumors (PNET) or a pancreatic ductal adenocarcinoma (PDAC)), stomach cancer, uterine cancer, and ovarian cancer.

In certain embodiments, the cancer is selected from, but not limited to, leukemias such as acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia (T-PLL), large granular lymphocytic leukemia, adult T-cell leukemia, and acute monocytic leukemia (AMOL).

In one embodiment, the cancer is a solid tumor that is associated with ascites. Ascites is a symptom of many types of cancer and can also be caused by a number of conditions, such as advanced liver disease. The types of cancer that are likely to cause ascites include, but are not limited to, cancer of the breast, lung, large bowel (colon), stomach, pancreas, ovary, uterus (endometrium), peritoneum and the like. In some embodiments, the solid tumor associated with ascites is selected from breast cancer, colon cancer, pancreatic cancer, stomach, uterine cancer, and ovarian cancer. In some embodiments, the cancer is associated with pleural effusions, e.g., lung cancer.

Additional hematological cancers that give rise to solid tumors include, but are not limited to, non-Hodgkin lymphoma (e.g., diffuse large B cell lymphoma, mantle cell lymphoma, B lymphoblastic lymphoma, peripheral T cell lymphoma and Burkitt's lymphoma), B-lymphoblastic lymphoma; B-cell chronic lymphocytic leukemia/small lymphocytic lymphoma; lymphoplasmacytic lymphoma; splenic marginal zone B-cell lymphoma (+villous lymphocytes); plasma cell myeloma/plasmacytoma; extranodal marginal zone B-cell lymphoma of the MALT type; nodal marginal zone B-cell lymphoma (+monocytoid B cells); follicular lymphoma; diffuse large B-cell lymphomas; Burkitt's lymphoma; precursor T-lymphoblastic lymphoma; T adult T-cell lymphoma (HTLV 1-positive); extranodal NK/T-cell lymphoma, nasal type; enteropathy-type T-cell lymphoma; hepatosplenic γ-δ T-cell lymphoma; subcutaneous panniculitis-like T-cell lymphoma; mycosis fungoides/sezary syndrome; anaplastic large cell lymphoma, T/null cell, primary cutaneous type; anaplastic large cell lymphoma, T-/null-cell, primary systemic type; peripheral T-cell lymphoma, not otherwise characterized;

angioimmunoblastic T-cell lymphoma, multiple myeloma, polycythemia vera or myelofibrosis, cutaneous T-cell lymphoma, small lymphocytic lymphoma (SLL), marginal zone lymphoma, CNS lymphoma, immunoblastic large cell lymphoma, precursor B-lymphoblastic lymphoma and the like.

Masked antibodies as described herein can also be used to treat disorders associated with cancer, e.g., cancer-induced encephalopathy.

The masked antibodies can be used in methods of treatment in combination with other therapeutic agents and/or modalities. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive (i.e., a synergistic response). The delivery can be such that an effect of the first treatment delivered is still detectable when the second is delivered.

In one embodiment, the methods of the invention include administering to the subject a masked antibody as described herein, e.g., in combination with one or more additional therapies, e.g., surgery or administration of another therapeutic preparation. In one embodiment, in the case of cancer, for example, the additional therapy may include chemotherapy, e.g., a cytotoxic agent. In one embodiment the additional therapy may include a targeted therapy, e.g. a tyrosine kinase inhibitor, a proteasome inhibitor, or a protease inhibitor. In one embodiment, the additional therapy may include an anti-inflammatory, anti-angiogenic, anti-fibrotic, or anti-proliferative compound, e.g., a steroid, a biologic immunomodulatory, such as an inhibitor of an immune checkpoint molecule, a monoclonal antibody, an antibody fragment, an aptamer, an siRNA, an antisense molecule, a fusion protein, a cytokine, a cytokine receptor, a bronchodilator, a statin, an anti-inflammatory agent (e.g. methotrexate), or an NSAID. In another embodiment, the additional therapy could include combining therapeutics of different classes. The antibody or masked antibody preparation and the additional therapy can be administered simultaneously or sequentially.

An “immune checkpoint molecule,” as used herein, refers to a molecule in the immune system that either turns up a signal (a stimulatory molecule) or turns down a signal (an inhibitory molecule). Many cancers evade the immune system by inhibiting T cell signaling. Hence, these molecules may be used in cancer treatments as additional therapeutics. In other cases, a masked antibody may be an immune checkpoint molecule.

Exemplary immune checkpoint molecules include, but are not limited to, programmed cell death protein 1 (PD-1), programmed death-ligand 1 (PD-L1), PD-L2, cytotoxic T lymphocyte-associated protein 4 (CTLA-4), T cell immunoglobulin and mucin domain containing 3 (TIM-3), lymphocyte activation gene 3 (LAG-3), carcinoembryonic antigen related cell adhesion molecule 1 (CEACAM-1), CEACAM-5, V-domain Ig suppressor of T cell activation (VISTA), B and T lymphocyte attenuator (BTLA), T cell immunoreceptor with Ig and ITIM domains (TIGIT), leukocyte-associated immunoglobulin-like receptor 1 (LAIR1), CD160, TGFR, adenosine 2A receptor (A2AR), B7-H3 (also known as CD276), B7-H4 (also called VTCN1), indoleamine 2,3-dioxygenase (IDO), 2B4, killer cell immunoglobulin-like receptor (KIR), and the like.

An “immune checkpoint inhibitor,” as used herein, refers to a molecule (e.g., a small molecule, a monoclonal antibody, an antibody fragment, etc.) that inhibit and/or block one or more inhibitory checkpoint molecules.

Exemplary immune checkpoint inhibitors include, but are not limited to, the following monoclonal antibodies: PD-1 inhibitors such as pembrolizumab (Keytruda, Merck) and nivolumab (Opdivo, Bristol-Myers Squibb); PD-L1 inhibitors such as atezolizumab (Tecentriq, Genentech), avelumab (Bavencio, Pfizer), durvalumab (Imfinzi, AstraZeneca); and CTLA-1 inhibitors such as ipilimumab (Yervoy, Bristol-Myers Squibb).

Exemplary cytotoxic agents include anti-microtubule agents, topoisomerase inhibitors, antimetabolites, protein synthesis and degradation inhibitors, mitotic inhibitors, alkylating agents, platinating agents, inhibitors of nucleic acid synthesis, histone deacetylase inhibitors (HDAC inhibitors, e.g., vorinostat (SAHA, MK0683), entinostat (MS-275), panobinostat (LBH589), trichostatin A (TSA), mocetinostat (MGCD0103), belinostat (PXD101), romidepsin (FK228, depsipeptide)), DNA methyltransferase inhibitors, nitrogen mustards, nitrosoureas, ethylenimines, alkyl sulfonates, triazenes, folate analogs, nucleoside analogs, ribonucleotide reductase inhibitors, vinca alkaloids, taxanes, epothilones, intercalating agents, agents capable of interfering with a signal transduction pathway, agents that promote apoptosis and radiation, or antibody molecule conjugates that bind surface proteins to deliver a toxic agent. In one embodiment, the cytotoxic agent that can be administered with a preparation described herein is a platinum-based agent (such as cisplatin), cyclophosphamide, dacarbazine, methotrexate, fluorouracil, gemcitabine, capecitabine, hydroxyurea, topotecan, irinotecan, azacytidine, vorinostat, ixabepilone, bortezomib, taxanes (e.g., paclitaxel or docetaxel), cytochalasin B, gramicidin D, ethidium bromide, emetine, mitomycin, etoposide, tenoposide, vincristine, vinblastine, vinorelbine, colchicin, anthracyclines (e.g., doxorubicin or epirubicin) daunorubicin, dihydroxy anthracin dione, mitoxantrone, mithramycin, actinomycin D, adriamycin, 1-dehydrotestosterone, glucocorticoids, procaine, tetracaine, lidocaine, propranolol, puromycin, ricin, or maytansinoids.

Assessment of proteases in tissues can be monitored using a variety of techniques, including both those that monitor protease activity as well as those that can detect proteolytic activity. Conventional methods that can detect the presence of proteases in a tissue, which could include both inactive and active forms of the protease, include IHC, RNA sequencing, Western blot, or ELISA-based methods. Additional techniques can be used to detect protease activity in tissues, which includes zymography, in situ zymography by fluorescence microscopy, or the use of fluorescent proteolytic substrates. In addition, the use of fluorescent proteolytic substrates can be combined with immuno-capture of specific proteases. Additionally, antibodies directed against the active site of a protease can be used by a variety of techniques including IHC, fluorescence microscopy, Western blotting, ELISA, or flow cytometry (See, Sela-Passwell et al. Nature Medicine. 18:143-147. 2012; LeBeau et al. Cancer Research. 75:1225-1235. 2015; Sun et al. Biochemistry. 42:892-900. 2003; Shiryaev et al. 2: e80. 2013.)

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing and method steps.

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following examples, which are included for purposes of illustration only and are not intended to be limiting. All patents, patent applications and references described herein are incorporated by reference in their entireties for all purposes.

### EXAMPLES

### Example 1. Generation of Masked Antibodies

Vel masked antibody Ab1, rituximab, trastuzumab, and hB6H12.3 antibodies were generated in a similar manner. These antibodies were linked to Vel-IPV, such that the Vel coiled-coil domain blocks antigen binding. In the presence of a protease, the linker could be cleaved and the mask removed, thus enabling the antibody to bind its antigen. The sequences for VelA and VelB are show in SEQ ID NOs 1 and 2, respectively. The sequences for VelA and VelB, including amino-terminal sequence QGASTT (SEQ ID NO: 138) and QGASTS (SEQ ID NO: 139), respectively, are shown in SEQ ID NOs: 3 and 4, respectively. VelA is linked through a cleavable linker to the amino-terminus of the light chain variable region and VelB is linked through a cleavable linker to the amino-terminus of the heavy chain variable region, unless indicated otherwise.

Antibodies were expressed via transient transfection of Expi HEK or Expi CHO cells or stable transfection of CHO-DG44 and purified using MabSelect SuRe columns (GE Healthcare). Additional preparative size-exclusion chromatography purification using Superdex columns (GE Healthcare) was performed for masked antibodies that were less than 90% monomeric. The identity and purity of each antibody was confirmed using liquid chromatography-mass spectrometry.

### Example 2. Analysis of Antibody Aggregation

The stability of antibodies was analyzed by analytical size-exclusion chromatography (SEC). A first analysis was the degree of antibody aggregation when incubated at room temperature.

The extent of aggregation of masked antibodies was also evaluated across different pH and salt conditions using solubility and stability screens (Hampton Research, Cat #HR2-072). In particular, an antibody was tested for aggregation after incubation with a variety of buffer and salt conditions contained within solubility and stability screen.

Antibody was added to each well of a 96-well polypropylene plate at a concentration of 2.25 mg/mL formulated in PBS buffer. To an antibody solution was added 20% v/v of each component of the Solubility and Stability Screen 2. The plate was sealed and incubated at room temperature for 96 hours.

The extent of aggregation of the conjugates was determined by SEC using an analytical SEC column (Sepax SRT-C 300 7.8 mm ID×30 cm, 5 μm) on a Waters 2695 HPLC system. The injected material was eluted using an isocratic mixture of 92.5% 25 mM sodium phosphate (pH 6.8), 350 mM NaCl, and 7.5% isopropyl alcohol at a flow rate of 1 mL/min

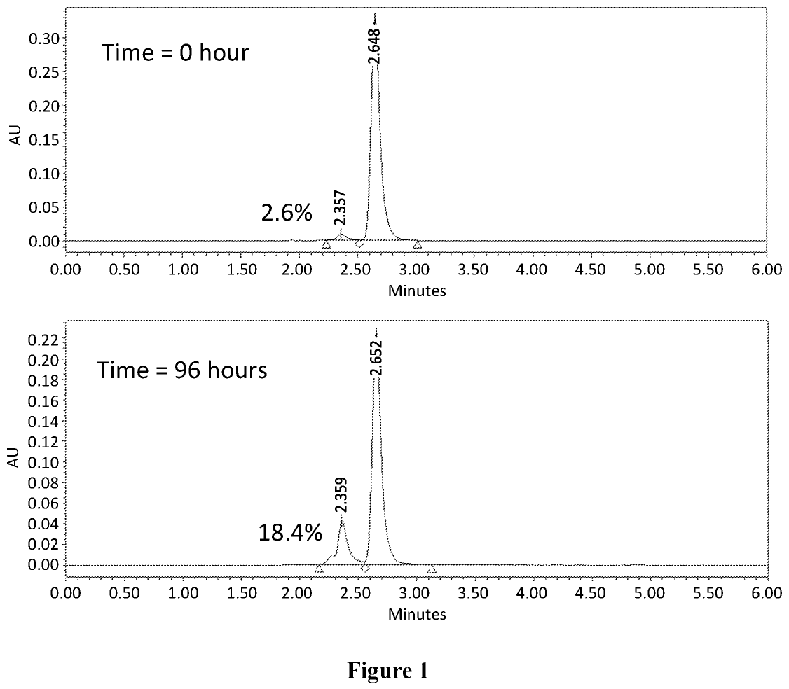

The starting aggregate for the Vel-IPV-Ab1 antibody was 2.6% (FIG. 1). After 96 hours, the control sample (where only distilled water was added) had 18.4% aggregate (i.e., 18.4 percent high molecular weight [HMW] species). Thus, Vel-IPV-Ab1 aggregation increased over time when incubated at room temperature at a concentration of 2.25 mg/mL.

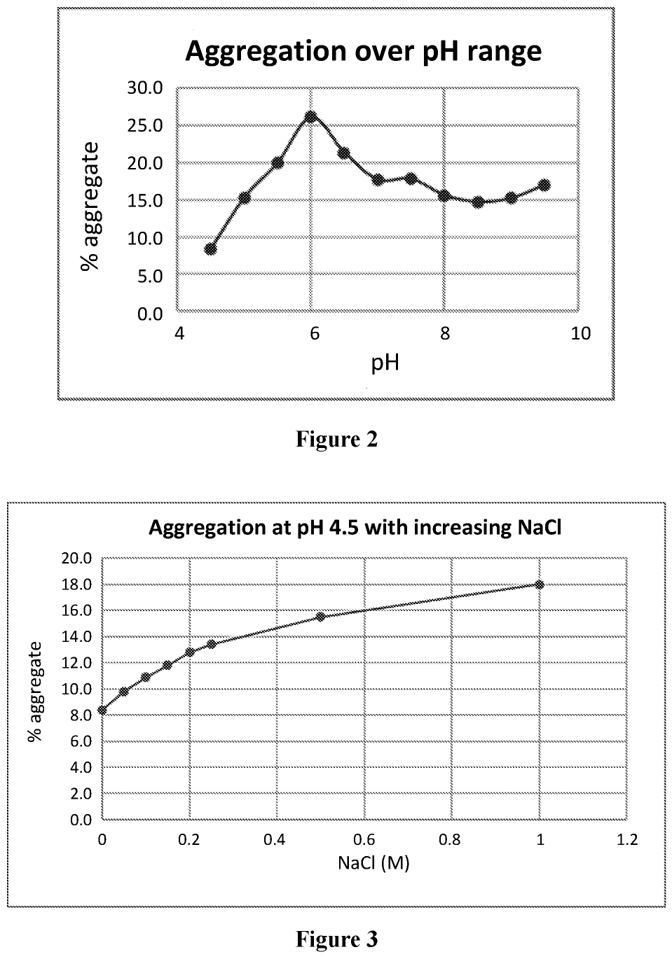

The pH dependence of aggregation was also evaluated using analytical SEC. The Vel-IPV-Ab1 antibody showed pH-dependent aggregation. The antibody aggregated the least at pH 4.5, with increasing aggregation up to pH 6, then slightly decreasing at elevated pH values (FIG. 2).

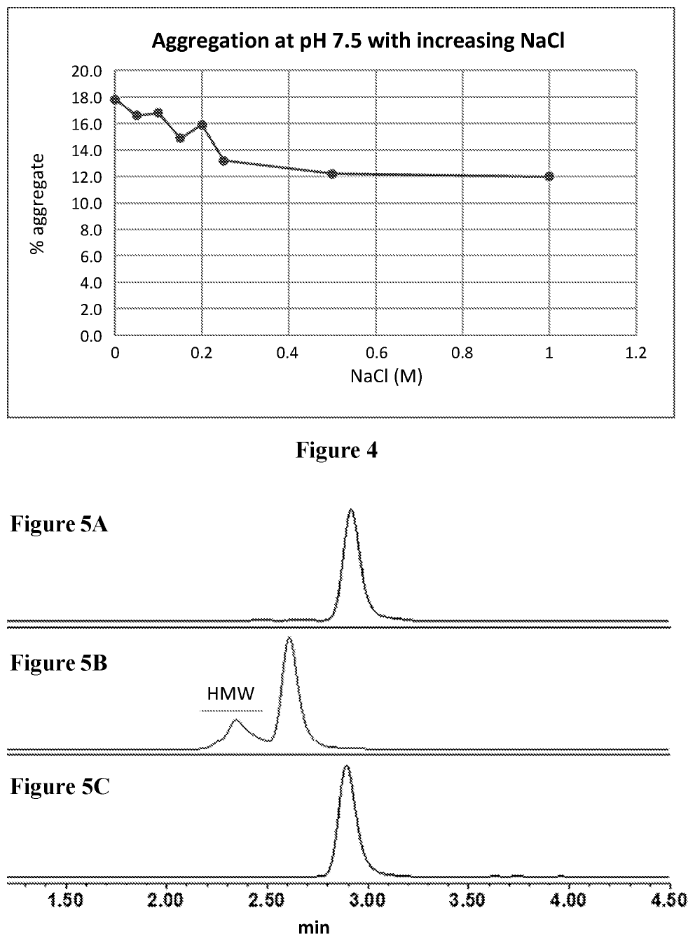

The effect was salt concentration was next determined. Increasing levels of NaCl from 0 to 1 M in sodium acetate buffer at pH 4.5 led to an increase in Vel-IPV-Ab1 aggregation following a 4-day incubation at room temperature (FIG. 3). Conversely, increasing concentrations of NaCl from 0 to 1 M in 0.05 M HEPES at pH 7.5 led to a decrease in antibody aggregation (FIG. 4).

These data suggest that aggregation of a Vel-masked antibody is based on buffer composition, pH, and salt composition.

### Example 3. Analysis of Masking Moiety Role in Aggregation

The effect of demasking of Vel-IPV-Ab1 was evaluated.

Vel-IPV-Ab 1 was treated with recombinant human MMP2 and assessed for aggregation upon mask cleavage. Recombinant MMP2 (60 pmol/min activity) was activated via incubation with 1.25 mM 4-aminophenyl mercuric acetate (APMA) for 1-2 hours at 37° C. Then, 1-2 μg of activated recombinant human (rh)MMP2 was added to 0.25-0.5 mg of masked antibody and incubated for 40 minutes at 37° C.

The demasked Vel-IPV-Ab1 was compared with masked Vel-IPV-Ab1 and with the Abl antibody (lacking any mask) using SEC analysis.

The control unmasked Ab1 antibody did not show high molecular weight (HMW) aggregated species (FIG. 5A). The masked Vel-IPV-Ab1 antibody showed HMW aggregated species without MMP2 treatment (FIG. 5B). The high molecular weight aggregated species are due to the presence of the Vel-IPV mask, as the demasked cleaved product following incubation of Vel-IPV-Ab1 with MMP2 did not have any associated high molecular weight species (FIG. 5C). This analysis demonstrates that the aggregation of Vel-IPV-Ab1 is driven by the Vel-IPV mask, as removal of the mask by MMP cleavage results in a monomeric antibody species without HMW species.

### Example 4. Antibody-Drug Conjugates

The aggregation of antibody drug conjugates (ADCs) was also evaluated.

ADCs were prepared by reduction of antibody interchain disulfides followed by addition of a 25-100% excess maleimide as described previously. (R. P. Lyon, D. L. Meyer, J. R. Setter, P. D. Senter, Methods Enzymol. 2012, 502, 123-138.) Full reduction of 8 thiols per antibody was accomplished by addition of 12 equivalents of tris (2-carboxyethyl)-phosphine (TCEP) to an antibody solution (1-10 mg/mL in PBS, pH 7.4). The extent of antibody reduction was monitored by reverse-phase LC-MS and additional TCEP was added as needed to complete the reaction. TCEP was then removed by ultrafiltration (3-times, 10-fold dilution into PBS, pH 7.4 containing 1 mM EDTA, centrifugation at 4000×g through a 30-kDa MWCO filter). Fully reduced antibodies in PBS-EDTA were conjugated with 10-16 molar equivalents (25-100% excess) of drug-linker or drug-carrier as a 10 mM DMSO stock.

The drug linker used for these experiments was MDpr-PEG12-glucuronide-PAB-MMAE (see Burke et al., Mol. Cancer Ther. 2017; 16 (1): 116-123).

The resulting solution after conjugation was vortexed and left at room temperature for 10-20 minutes. The extent of conjugation was assessed by reverse-phase LC-MS as described below, and additional drug-linker or drug-carrier was added as needed. Once all available Cys thiols were alkylated, the crude ADC solution was purified by buffer exchange into PBS using either a Nap-5 desalting column (GE Healthcare) or through 3-5 rounds of ultrafiltration. The final ADC concentration was determined spectrophotometrically.

LC-MS was performed by reduced antibody reverse-phase LC-MS using a Waters Acquity/Xevo UPLC equipped with a PLRP-MS 3 μm column (Agilent). Samples were reduced with 10 mM dithiothreitol (DTT) for 10 min at 37° C. and then chromatographed over an analytical reversed-phase column (Agilent Technologies, PLRP-S, 300 Å, 2.1 mm ID×50 mm, 3 μm) at 80° C. and eluted with a linear gradient of 0.01% TFA in acetonitrile from 25% to 65% in 0.05% aqueous TFA over 5 minutes, followed by isocratic 65% 0.01% TFA in acetonitrile for 0.5 min at a flow rate of 1.0 mL/min. Mass spectrometry data was acquired in ESI+ mode using a mass range of 500-4000 m/z and were deconvoluted using MaxEnt1 to determine masses of the resulting conjugates. Data was analyzed using UNIFI software (Waters).

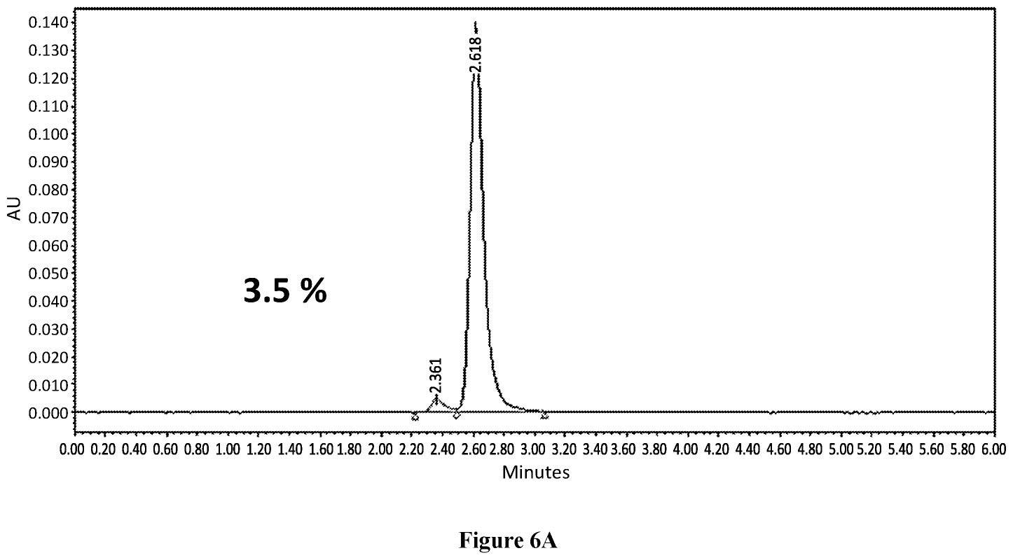

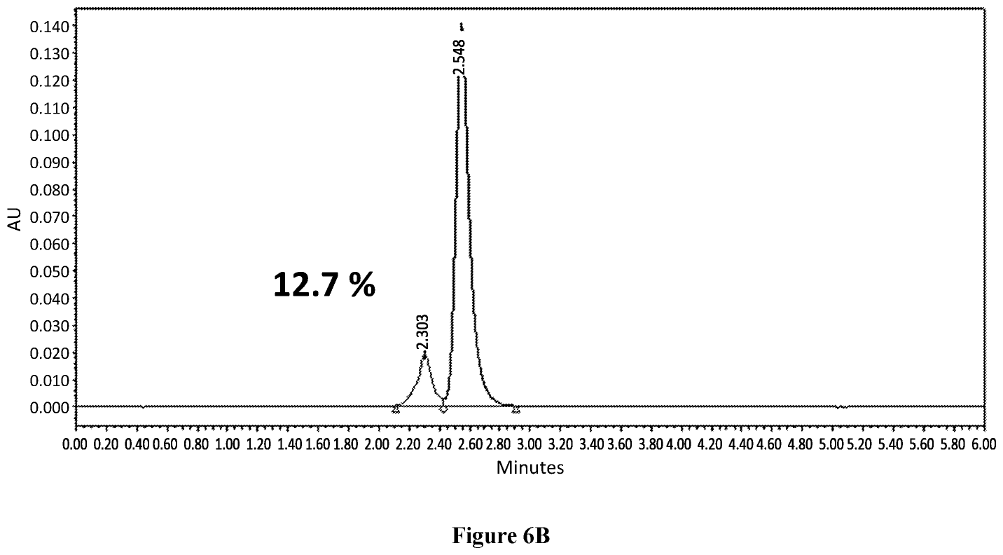

Vel-IPV-Ab1 antibody-drug conjugates bearing MDpr-PEG12-glucuronide-PAB-MMAE drugs, at a loading of 8 drugs per antibody, have increased aggregation compared to naked (i.e., unconjugated) antibody. The drug conjugation procedure described above requires antibody interchain disulfide reduction and drug conjugation at elevated pH, which may result in an ADC with increased aggregation (FIG. 6B) as measured by percentage high HMW species compared to the unconjugated antibody (FIG. 6A). Thus, drug conjugation of SEC-purified Vel-IPV-Ab1 resulted in an increase in aggregation of HMW species of the ADC compared to unconjugated Vel-IPV-Ab1 antibody.

### Example 5. Mutagenesis Analysis to Identify Coiled-Coil Peptides with Improved Stability

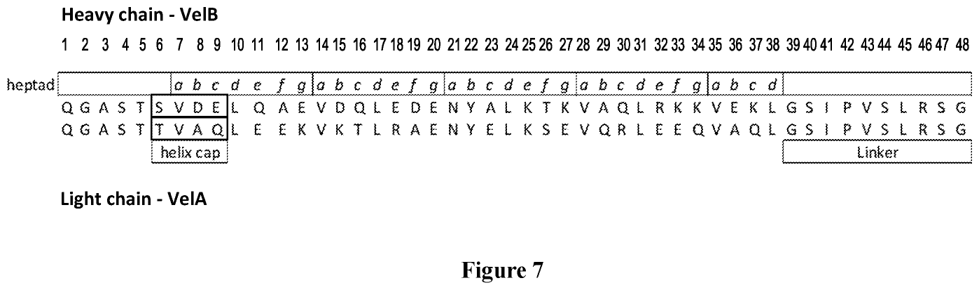

A mutagenesis campaign was conducted to identify Vel coiled-coil masking domains with improved stability and decreased aggregation. Single point mutations in either the VelA (light chain) or VelB (heavy chain) sequence, or paired mutations in both the light chain and heavy chain at opposing sites within the heterodimer, were made in Vel-IPV-Ab1. VelA and VelB sequences are presented in FIG. 7.

The mutants were aimed at modulating the inter-coil affinity to reduce homodimerization of the same peptide on different antibody Fab arms or to better balance the charge and hydrophobicity of the coiled-coil domain. The initial set of antibody variants was produced on a 1 mL culture scale in Expi-293 cells and purified using MabSelect SuRE Protein A resin. Antibodies were eluted from Protein A resin using a buffer containing 20 mM glycine, pH 3, then neutralized with 10% v/v 800 mM potassium phosphate buffer, 500 mM NaCl, 50 mM EDTA, pH 8.0. The extent of aggregation was assessed by analytical SEC.

Table 2 presents a summary of aggregation properties of different Vel-IPV-Ab1 antibody variants. ΔQ1 refers to a variant lacking the first amino acid of VelB or VelA.

TABLE 2
Vel-IPV-Ab1 antibody variants produced

| | | | % HMW |
|---|---|---|---|
| Coil | | | (Protein A |
| No. | Heavy chain | Light chain | elution) |
| 1 | Wild-type (WT) | Wild-type (WT) | 50.0 |
| | VelB (SEQ ID | VelA (SEQ ID | |
| | NO: 2) | NO: 1) | |
| 2 | ΔQ1 | ΔQ1 | 28.8 |
| 3 | V7N | V7N | 46.0 |
| 4 | V14A | V14I | 27.0 |
| 5 | V14I | V14A | 38.7 |
| 6 | V14N | V14N | 40.6 |
| 7 | L17I | L17A | 14.6 |
| 8 | Y22C | Y22C | 38.2 |
| 9 | Y22S | Y22S | 29.5 |
| 10 | L24I | L24A | 10.0 |
| 11 | V28A | V28I | 21.9 |
| 12 | V28I | V28A | 17.5 |
| 13 | V28N | V28N | 17.9 |
| 14 | L31I | L31A | 7.6 |
| 15 | V35A | V35I | 24.9 |
| 16 | V35I | V35A | 19.8 |
| 17 | V35N | V35N | 28.3 |
| 18 | D8Q | WT | 25.9 |
| 19 | D8N | WT | 22.5 |
| 20 | E9N | WT | 35.4 |
| 21 | E9Q | WT | 33.0 |
| 22 | E13R | WT | 34.3 |
| 23 | E13N | WT | 44.1 |
| 24 | E13Q | WT | 27.9 |
| 25 | D15E | WT | 28.2 |
| 26 | D15S | WT | 22.6 |
| 27 | D15N | WT | 22.6 |
| 28 | D19S | WT | 40.6 |
| 29 | D19N | WT | 23.8 |
| 30 | E20K | WT | 46.8 |
| 31 | E20N | WT | 58.2 |
| 32 | E20Q | WT | 46.6 |
| 33 | R32K | WT | 30.9 |
| 34 | K33Q | WT | 41.4 |
| 35 | K33E | WT | 30.6 |
| 36 | K37Q | WT | 20.1 |
| 37 | K37E | WT | 23.7 |
| 38 | D8K | WT | 19.8 |
| 39 | D8H | WT | 22.9 |
| 40 | E9K | WT | 33.0 |
| 41 | E9H | WT | 34.9 |
| 42 | Q11K | WT | 36.6 |
| 43 | Q11H | WT | 51.9 |
| 44 | A12K | WT | 21.3 |
| 45 | A12H | WT | 36.4 |
| 46 | D15K | WT | 20.6 |
| 47 | D15H | WT | 31.5 |
| 48 | D19K | WT | 33.1 |
| 49 | D19H | WT | 26.1 |
| 50 | K25H | WT | 43.4 |
| 51 | K34H | WT | 38.8 |
| 52 | E36H | WT | 11.5 |
| 53 | K25R | WT | 33.3 |
| 54 | WT | E11Q | 32.4 |
| 55 | WT | E11N | 59.1 |
| 56 | WT | E11K | 44.5 |
| 57 | WT | E12Q | 48.7 |
| 58 | WT | E12N | 64.8 |
| 59 | WT | E12K | 54.0 |
| 60 | WT | T16A | 40.2 |
| 61 | WT | WT | 36.7 |
| 62 | ΔQ1 | ΔQ1 | 26.7 |
| 63 | WT | E20Q | 51.3 |
| 64 | WT | E20N | 67.7 |
| 65 | WT | E20K | 61.0 |
| 66 | WT | E23Q | 34.8 |
| 67 | WT | E32Q | 46.4 |
| 68 | WT | E32N | 57.9 |
| 69 | WT | E32K | 40.3 |
| 70 | WT | E33Q | 34.6 |
| 71 | WT | E33N | 37.8 |
| 72 | WT | E33K | 24.1 |
| 73 | WT | A8H | 52.8 |
| 74 | WT | A8K | 46.7 |
| 75 | WT | Q9H | 58.8 |
| 76 | WT | Q9K | 58.1 |
| 77 | WT | E11H | 63.0 |
| 78 | WT | E12H | 57.3 |
| 79 | WT | K15H | 65.1 |
| 80 | WT | K15D | 66.3 |
| 81 | WT | A19H | 55.7 |
| 82 | WT | K25H | 65.0 |
| 83 | WT | E33H | 25.2 |
| 84 | WT | A36H | 43.9 |
| 85 | WT | K25R | NA |

Under these conditions, the wild-type (WT) Vel-IPV-Ab1 was highly aggregated (50, 37.6% HMW), whereas many of the mutants had improved aggregation properties, with some exhibiting less than 10% HMW. Conversely, mutation of many residues, particularly in the light chain, resulted in >50% HMW. Relatively lower aggregation was observed with mutations to both the light and heavy chain (Light+Heavy). Paired mutations at the leucine zipper interface (for example, mutation of Leu-Leu or Val-Val at opposing sites to Ala-Ile) consistently showed improved aggregation properties in this screen.

Next, aggregation properties of different Vel-IPV-Ab1 variants was assessed over room temperature incubations. A set of clones with beneficial mutations were expressed on a 30 mL scale in Expi-293 cells, Protein A purified, buffer exchanged into PBS, pH 7.4, concentration normalized to 2.1 mg/mL, and evaluated for stability in protein A eluate either after PBS exchange (t=0d) or following room temperature incubation for 7 days in PBS (t=7d), as shown in Table 3. Some variants that displayed improved properties in 1 mL high-throughput expression did not show preferential aggregation properties upon more normalized comparisons. Variants with improved profiles include those with mutations to only the heavy chain or with mutations to both the heavy chain and light chain.

TABLE 3
Further stability analysis of Vel-IPV-Ab1 antibody variants

| | | | % HMW | % HMW | % HMW |
|---|---|---|---|---|---|
| Coil | Heavy chain | Light chain | Protein A | PBS | PBS |
| No. | (VelB) | (VelA) | elute | t = 0 d | t = 7 d |
| 1 | Wild-type | Wild-type | 10.5 | 22.3 | 35.2 |
| | (WT) VelB | (WT) VelA | | | |
| 2 | ΔQ1 | ΔQ1 | 8.6 | 21.6 | 39.3 |
| 10 | L24I | L24A | 1.9 | 2.0 | 2.3 |
| 14 | L31I | L31A | 8.9 | 17.8 | 10.6 |
| 16 | V35I | V35A | 2.3 | 11.9 | 23.7 |
| 38 | D8K | WT | 5.2 | 15.6 | 39.1 |
| 52 | E36H | WT | 5.7 | 5.6 | 5.4 |

Experiments were also done to assess combinations of beneficial mutations in the light and heavy chain of Vel-IPV-Ab1. The stability of WT and mutated Vel-IPV-Ab1 antibodies was assessed after 30 mL expression under normalized concentration conditions in PBS, pH 7.4 buffer. In these experiments, beneficial mutations in the light and heavy chains were combined (see Coils 86-99) and their stability compared to Coils 10 and 14.

TABLE 4
Stability analysis of additional Vel-IPV-Ab1 antibody variants,
including combinations of light and heavy mutations

| | | | | % HMW | % HMW |
|---|---|---|---|---|---|
| | Heavy | Light | % HMW | PBS | PBS |
| Coil | chain | chain | Protein A | t = 0 d, | t = 7-8 d, |
| No. | (VelB) | (VelA) | elute | 2.6 mg/mL | 2.6 mg/mL |
| 2 | ΔQ1 | ΔQ1 | 6.8 | 14.6 | 41.7 |
| 10 | L24I | L24A | 1.1 | 1.8 | 2.0 |
| 14 | L31I | L31A | 15.4 | 16.0 | 9.8 |
| 86 | E36H | L24A | 12.4 | 13.2 | 6.4 |
| 87 | E36H | L31A | 10.1 | 20.6 | 14.1 |

TABLE 5
Stability analysis of additional Vel-IPV-Ab1 antibody variants,
including multiple mutations in the heavy and/or light chain

| | | | % HMW | % HMW |
|---|---|---|---|---|
| | | | PBS | PBS |
| Coil | Heavy chain | Light chain | t = 0 d, | t = 7 d, |
| No. | (VelB) | (VelA) | 2.3 mg/mL | 2.3 mg/mL |
| 88 | L24I/L31I | L24A/L31A | 1.5 | 1.2 |
| 89 | L17I/L31I | L17A/L31A | 1.6 | 1.2 |
| 90 | L17I//L24I/L31I | L17A//L24A/L31A | 1.3 | 1.0 |
| 91 | L31I/V35I | L31A/V35A | 11.1 | 12.2 |
| 92 | V28I/L31I | V28A/L31A | 0.0 | 1.9 |
| 93 | V28I/L31I/V35I | V28A/L31A/V35A | 57.9 | 42.8 |
| 94 | L24I/V28I/L31I | L24A/V28A/L31A | 100 | 100 |
| 95 | L31I | L31A/E33K | 1.1 | 0.9 |
| 96 | D8K/E36H | WT | 2.5 | 3.1 |
| 97 | D8K/E36H/L31I | L31A | 2.5 | 0.7 |
| 98 | D8K/D15K/L31I | L31A | 0.5 | 1.0 |
| 99 | D15K/E36H/L31I | L31A | 1.0 | 0.8 |

As shown in Tables 4 and 5, some Vel-IPV-Ab1 antibody variants with combinations of mutations showed dramatically improved stability profiles with less than 5% HMW even after incubation for 7 days at room temperature. However, other Vel-IPV-Ab1 antibody variants with combinations of mutations showed extremely high aggregation levels (e.g., Coil 94 with multiple mutations in the light and heavy chain that had been beneficial as single mutations). Thus, while some combined mutations provided benefits in stability, some instead resulted in highly aggregated antibodies.

Further variants of Vel-IPV-Ab1 were made and expressed in Expi-293 where the mutations to Ile or Ala were transposed to the opposing antibody chain to assess whether the benefits of the mutations at the L24 and L31 mutation are chain-specific. For example, the variant with mutations of light chain L24A and heavy chain L24I variant (Coil 10) was used to design a variant with mutations of light chain L24I and heavy chain L24A modifications (Coil 101). In addition, a variant of Vel-IPV-Ab1 was expressed where the heterodimeric peptides of the coiled-coil domain were switched from light to heavy chain and vice versa (i.e. VelA peptide on the heavy chain and VelB peptide on the light chain, Coil 100). These variants were expressed on 1 mL scale in Expi-293 and assessed for stability at 1 mg/mL in PBS for 9 days. While switching the coiled-coil domains of the light and heavy chains did not impart a significant effect, the mutations in Coils 101 and 102 improved antibody stability, as shown in Table 6.

TABLE 6
Stability characteristics of selected coiled-coil mutations
or switched heterodimeric peptides on Vel-IPV-Ab1

| | | | | % HMW | % HMW |
|---|---|---|---|---|---|
| | | | % HMW | PBS | PBS |
| Coil | Heavy | Light | Protein A | t = 0 d, | t = 9 d, |
| No. | chain | chain | elute | 1.0 mg/mL | 1.0 mg/mL |
| 100 | Switch | Switch | 31.6 | 46.5 | 34.5 |
| | (VelA) | (VelB) | | | |
| 101 | L24A | L24I | 0.8 | 1.1 | 1.4 |
| | (VelB) | (VelA) | | | |
| 102 | L31A | L24I | 5.4 | 9.2 | 6.7 |
| | (VelB) | (VelA) | | | |

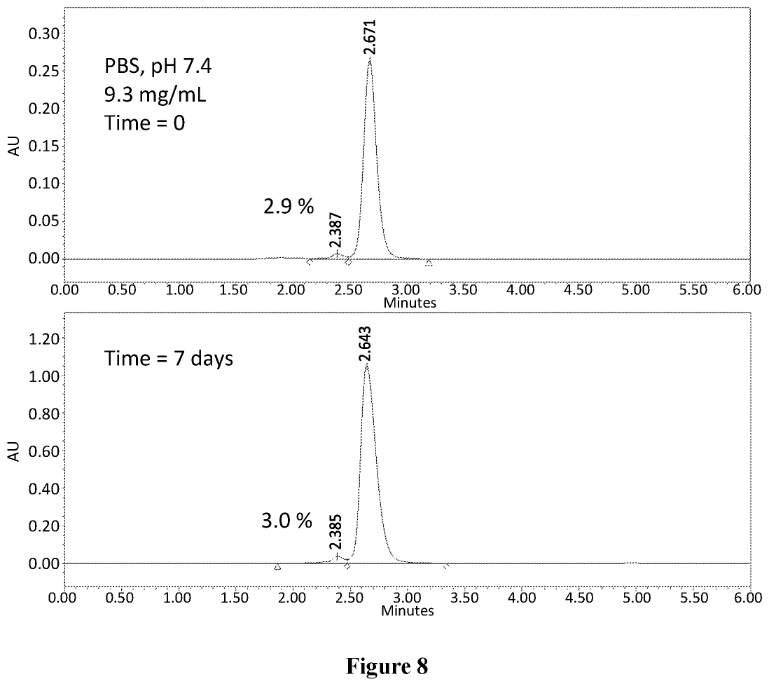

The Vel-IPV-Ab1 variant bearing light chain L24A and heavy chain L24I mutations (Coil 10) was formulated in PBS, pH 7.4 at a concentration of 9.3 mg/mL, and its stability assessed by analytical SEC after incubation at room temperature for 7 days. The high molecular weight species increased slightly upon incubation at room temperature for 7 days, as the initial percentage HMW was 2.9% and the percentage HMW after 7 days was 3.0% (FIG. 8). These data indicate that light chain L24A and heavy chain L24I mutations improve Vel-IPV-Ab1 stability.

Several clones were expressed on 30 mL scale in a separate expression host, Expi-CHO cells and assessed for differences in behavior from expression in Expi-293. Antibodies were purified in the same manner as previous, and formulated to 2.0 mg/mL in PBS. A similar benefit in aggregation stability was observed for the antibodies bearing dual modifications at the L24 or L31 sites, as shown in Table 7.

TABLE 7
Stability characteristics of selected coiled-coil
mutations on Vel-IPV-Ab1 expressed in Expi-CHO.

| | | | % HMW | % HMW |
|---|---|---|---|---|
| Coil | Heavy chain | Light chain | PBS t = 0 d, | PBS t = 7 d, |
| No. | (VelB) | (VelA) | 2.0 mg/mL | 2.0 mg/mL |
| 1 | WT | WT | 38.9 | 36.5 |
| 2 | ΔQ1 | ΔQ1 | 35.3 | 36.4 |
| 10 | L24I | L24A | 5.6 | 5.7 |
| 14 | L31I | L31A | 21.4 | 16.5 |
| 38 | D8K | WT | 43.9 | 39.2 |
| 52 | E36H | WT | 39.9 | 27.5 |

### Example 6. Analysis of Vel Coiled-Coil Mutations in Other Antibodies

A set of the Vel coiled-coil mutations were applied to masking of three additional antibodies: hB6H12.3 targeting CD47, trastuzumab targeting HER2, and rituximab targeting CD20. Antibodies were incubated in PBS up to either 9 days (rituximab and trastuzumab) or 11 days (hB6H12.3). The results are presented in Tables 8-10.

TABLE 8
Stability characteristics of selected coiled-coil
mutations on Vel-IPV-hB6H12.3 expressed in Expi-293

| | | % HMW | % HMW | % HMW | % HMW |
|---|---|---|---|---|---|
| Heavy | Light | PBS t = | PBS t = | PBS t = | PBS t = |
| chain | chain | 0 d, 2.0 | 7 d, 2.0 | 0 d, 12.1 | 11 d, 12.1 |
| (VelB) | (VelA) | mg/mL | mg/mL | mg/mL | mg/mL |
| WT | WT | 5.1 | 9.2 | 7.9 | 39.7 |
| ΔQ1 | ΔQ1 | 4.6 | 10.5 | 9.4 | 40.5 |
| L17I | L17A | 15.5 | 7.1 | 22.0 | 14.7 |
| L24I | L24A | 2.1 | 2.1 | 2.5 | 3.4 |
| L31I | L31A | 2.6 | 2.6 | 3.6 | 4.8 |
| L24I/L31I | L24A/L31A | 1.3 | 1.2 | 1.4 | 2.4 |
| D8K | WT | 4.0 | 8.0 | 6.8 | 21.2 |
| E36H | WT | 6.3 | 22.0 | 14.7 | 67.3 |
| Switch | Switch | 1.3 | 1.2 | 1.4 | 2.4 |
| (VelA) | (VelB) | | | | |

TABLE 9
Stability characteristics of selected coiled-coil mutations
on Vel-IPV-trastuzumab expressed in Expi-293

| | | % HMW PBS | % HMW |
|---|---|---|---|
| Heavy chain | Light chain | t = 0 d, 1.0 | PBS t = 9 d, |
| (VelB) | (VelA) | mg/mL | 1.0 mg/mL |
| WT | WT | 12.3 | 6.9 |
| L17I | L17A | 11.9 | 10.2 |
| L24I | L24A | 6.1 | 5.3 |
| L31I | L31A | 3.6 | 3.3 |
| L24I/L31I | L24A/L31A | 3.3 | 2.7 |
| V35I | V35A | 10.6 | 8.9 |
| D8K | WT | 9.3 | 5.7 |
| E36H | WT | 10.7 | 5.9 |

TABLE 10
Stability characteristics of selected coiled-coil mutations
on Vel-IPV-rituximab expressed in Expi-293

| | | % HMW | % HMW |
|---|---|---|---|
| Heavy chain | Light chain | PBS t = 0 d, | PBS t = 9 d, |
| (VelB) | (VelA) | 1.0 mg/mL | 1.0 mg/mL |
| WT | WT | 16.5 | 8.6 |
| L17I | L17A | 3.1 | 2.6 |
| L24I | L24A | 2.3 | 2.0 |
| L31I | L31A | 2.3 | 2.5 |
| L24I/L31I | L24A/L31A | 3.9 | 2.5 |
| V35I | V35A | 13.1 | 8.9 |
| D8K | WT | 13.9 | 8.6 |
| E36H | WT | 14.5 | 7.5 |

These data show that aggregation of a Vel-IPV-antibody is dependent on a number of factors, including the antibody used and the presence of different mutations in the heavy and light chains of Vel.

### Example 7. Analysis of ADCs with Vel-IPV-Ab1 Variants

Antibody-drug conjugates were prepared for Vel-IPV-Ab1 and selected variants to compare the extent of aggregation induced by the antibody reduction and drug-linker conjugation procedure.

Antibody interchain disulfide reduction was completed using 25 antibody equivalents of 10 mM TCEP for 60 min at 37° C. in PBS buffer at pH 8.0. Upon reduction, excess TCEP was removed by dilution and concentration using a 0.5 mL 30 kDa Amicon spin filter. Antibody was diluted and concentrated three times into PBS containing 2 mM EDTA. At this time, each antibody was incubated with 11 molar equivalents (per antibody) of MDpr-gluc-PEG12-MMAE drug-linker in PBS buffer containing 2 mM EDTA at pH 7.4 and allowed to react for 30 min at room temperature. Excess drug-linker was removed by incubation for 30 min with a charcoal slurry followed by filtration through 0.5 mL 0.22 μm filters. The conjugates were then buffer exchanged into PBS, pH 7.4 using 30 kDa molecular weight cutoff spin filters to an approximate concentration of 1-2 mg/mL.

In comparison to the wild-type Vel-IPV-Ab1 sequence, several of the variants had improved aggregation properties upon conjugation, as shown in Table 11. For example, aggregation of Coil 14 decreased during the conjugation procedure.

TABLE 11
Aggregation properties of selected Vel-
IPV-Ab1 variant antibody-drug conjugates

| | | | | Post- | |
|---|---|---|---|---|---|
| | | | Initial | reduction | Final |
| | Heavy chain | Light chain | Ab % | Ab % | ADC % |
| Coil No. | (VelB) | (VelA) | HMW | HMW | HMW |
| 1 | WT | WT | 3.5 | 6.2 | 12.7 |
| 10 | L24I | L24A | 1.5 | 1.3 | 2.5 |
| 14 | L31I | L31A | 12.3 | 3.8 | 6.8 |
| 92 | V28I/L31I | V28A/L31A | 3.0 | 1.2 | 2.6 |
| 88 | L24I/L31I | L24A/L31A | 1.0 | 0.6 | 2.4 |
| 38 | D8K | WT | 11.6 | 12.0 | 19.0 |
| 52 | E36H | WT | 2.9 | 2.6 | 6.9 |

These data show that Vel-IPV-Ab1 variants can be used to generate ADCs with acceptable stability.

### Example 8. Analysis of ELISA Binding of Vel-IPV-Ab1 Variants

The impact of Vel coiled-coil mutations on binding was assessed by saturation binding to antigen-positive cells and to recombinant antigen by ELISA.

Saturation binding ELISA experiments were performed. Soluble recombinant antigens were diluted to an appropriate concentration in 50 mM carbonate buffer, pH 9.6. To each well of a 96- or 384-well Maxisorb ELISA plate was added 100 μL of soluble antigen. The plate was sealed and stored at 4° C. overnight. The plate was then washed 3-5 times with PBS-T was buffer (PBS, pH 7.4+0.05% Tween-20). The wells were blocked using 300 μL/well of PBS-T buffer containing BSA for 1 hour at room temperature, then washed 3-5 times with PBS-T. Dilutions of antibodies were prepared in blocking buffer and added to each well in a volume of 100 μL. The antibodies were incubated for 1 hour at room temperature, and then washed 3-5 times with PBS-T. HRP-conjugated secondary antibodies (either anti-human Fc or anti-human kappa light chain) were then added and incubated for 1 hour at room temperature. The plate was washed 3-5 times with PBS-T. The ELISA was developed by adding 100 μL of TMB solution and incubating for 3-15 min at room temperature. To stop the reaction, 100 μL of 1 N sulfuric acid was added to each well. The absorbance at 450 nm was determined using a Spectramax 190 plate reader (Molecular Biosciences) and the data plotted using GraphPad Prism 6.

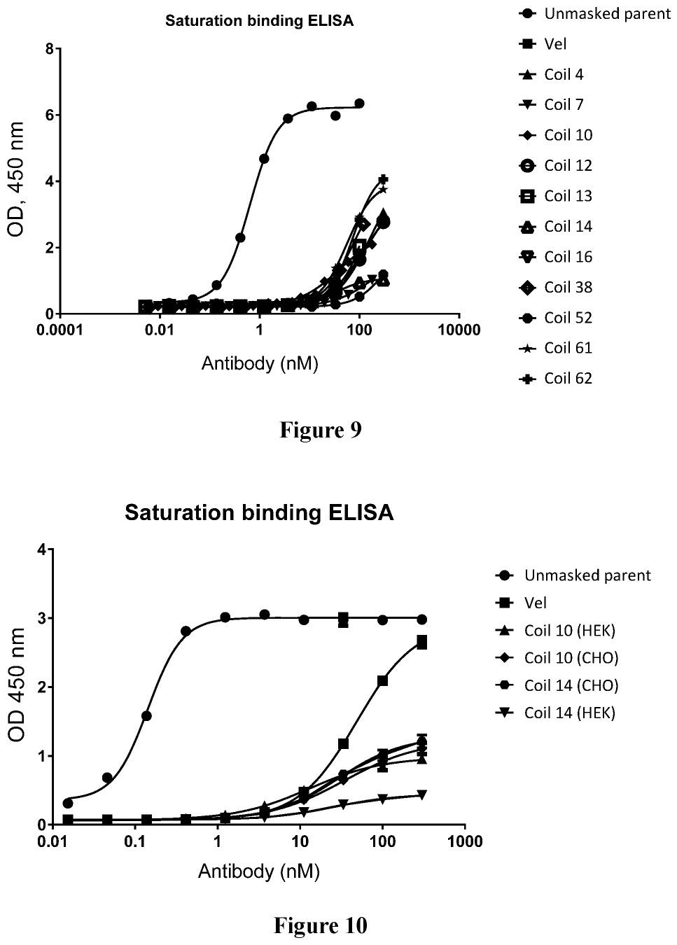

Saturation binding ELISA was performed with Ab1, Vel-IPV-Ab1, and a range of Vel-IPV-Ab1 variants (described in Table 2) against recombinant Ab1 target (FIG. 9). Vel-IPV-Ab1 Coil 10 and Coil 14 block antigen binding to a more significant extent than the wild-type Vel coiled-coil domain when these antibodies were expressed in either HEK or CHO cells (FIG. 10).

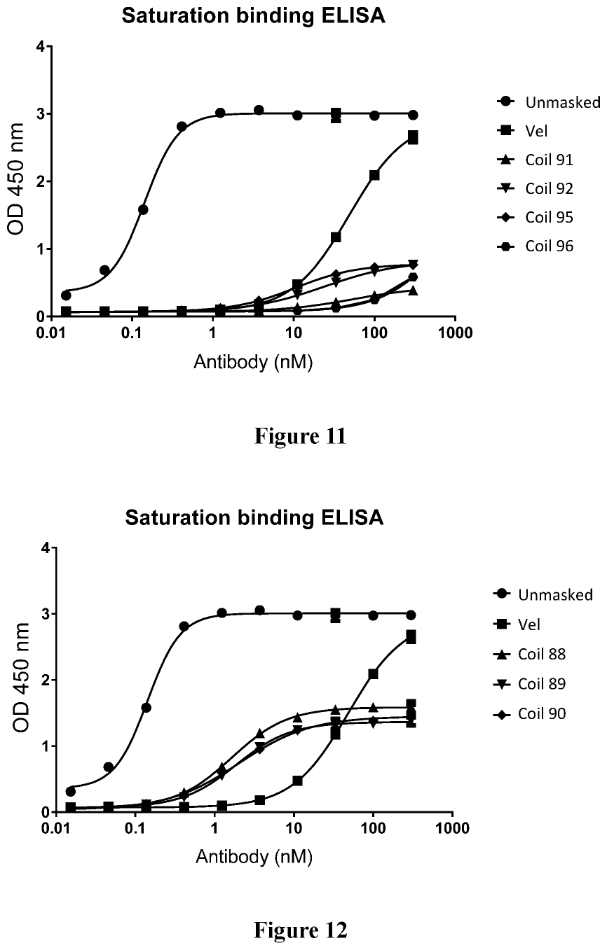

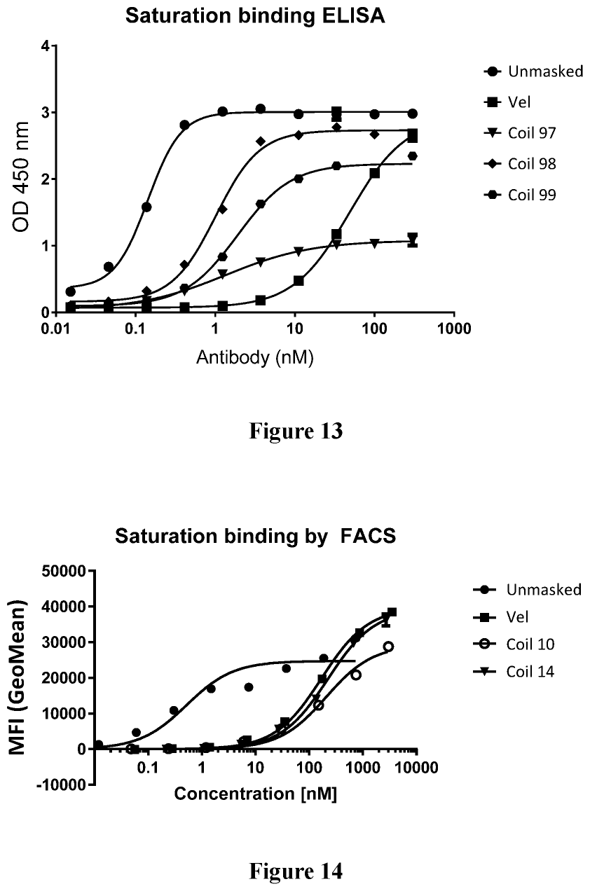

Additional variants of Vel-IPV-Ab1 were also analyzed by saturation binding ELISA. Saturation binding ELISA was performed with Ab1, Vel-IPV-Ab1, and Vel-IPV-Ab1 Coils 91, 92, 95, and 96 antibodies against recombinant Ab1 target. Coils 91, 92, 95, and 96 blocked antigen binding more than the wild-type Vel coiled-coil domain (FIG. 11). In contrast, Vel-IPV-Ab1 Coils 88-90 showed less block of antigen binding against recombinant Ab1 target compared to the wild-type Vel coiled-coil domain (FIG. 12). Vel-IPV-Ab1 Coils 97-99 showed a range of blocking ability, but generally decreased blocking compared to the wild-type Vel coiled-coil domain (FIG. 13).

These data indicate that mutations to specific amino acids in VelA and/or VelB can have a range of effects on a masked antibody.

### Example 9. Analysis of FACS Binding of Vel-IPV-Ab1 Variants

Select Vel-IPV-Ab1 variants were also assessed by FACS analysis.

For cellular binding analysis by FACS, cells expressing the target protein of interest (2×10⁵) were treated with a serial dilution of indicated antibody in staining buffer (PBS, 5% FBS, 0.2% NaN₃). Samples were incubated for 1 hour on ice and washed twice with ice-cold staining buffer. Cells were resuspended with anti-human IgG-AF488 or IgG-AF647 (JacksonImmunoResearch), 1:200 dilution in staining buffer) for 1 hour on ice. Cells were washed twice with ice cold staining buffer and resuspended in staining buffer. Labeled cells were examined by flow cytometry on an Invitrogen Attune N×T flow cytometer gated to exclude nonviable cells, and the data analyzed using FlowJo10 software. When determined, the Kd was calculated using GraphPad Prism 6 using non-linear regression.

Saturation binding on Ab1 target-expressing HEK-293F cells was analyzed with Ab1, Vel-IPV-Ab1, and Vel-IPV-Ab1 Coil 10 and Coil 14 antibodies (FIG. 14). All three Vel-masked antibodies display a similar decrease in binding to Ab1 target, suggesting that mutations in the coiled-coil domains in Coil 10 and Coil 14 did not affect the masking ability compared to wild-type Vel.

### Example 10. Cleavage Assessment of Vel-Masked Ab1 Antibodies

The ability of proteases to cleave different Vel masks was next assessed by measuring the demasking of Vel-IPV-Ab1 variants. Cleavage of Vel-masked Ab1 antibodies was assessed using normalized suboptimal matrix metalloprotease (MMP) activity.

To assess whether the Vel variants could still be efficiently cleaved by relevant proteases, Vel-IPV-Ab1 and Coils 10, 14, 88, 89, 90, 95, 96, 97, 98, and 99 were incubated with active, recombinant MMP-2 (60 pmol/min activity). Recombinant MMP was activated via incubation with 1.25 mM 4-aminophenyl mercuric acetate (APMA) for 1-2 hours at 37° C. The percent cleaved light and heavy chains was assessed after 40 min incubation at 37° C. using LC-MS.

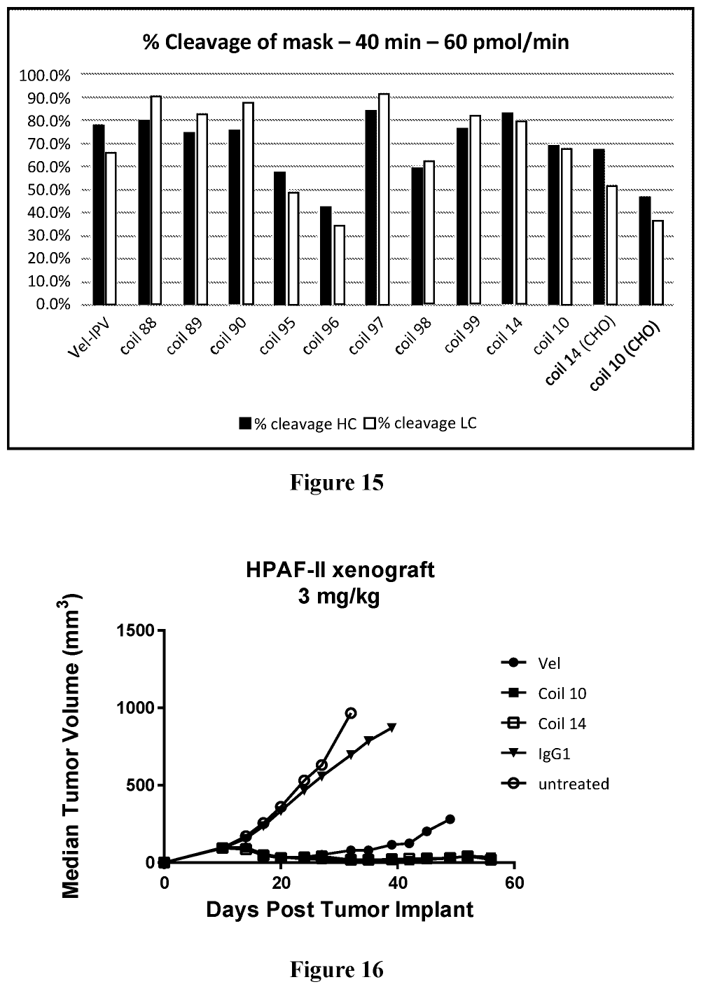

LC-MS analysis indicated that each Vel-IPV-Ab1 variants was cleaved to a similar extent as the parent Vel coiled-coil masked antibody (FIG. 15). Thus, the mutations to VelA and VelB did not seem to affect the ability to demask an antibody with MMP treatment.

### Example 11. In Vivo Evaluation of ADCs Comprising Vel-IPV-Ab1 Variants

The in vivo efficacy of Ab1 ADCs was evaluated in two xenograft models in nude mice (HPAF-II and BxPC3), which express the Ab1 target, at a dose of 3 mg/kg. The ADCs tested were DAR8 conjugates (i.e., drug-to-antibody ration of 8) of MDpr-gluc-PEG12-MMAE, generated as described in Example 4. To conduct the efficacy experiments, 5×10⁶ cells were injected subcutaneously into 5 female nude mice (Harlan) per group. Mice were randomly divided to study groups and dosed with test article via intraperitoneal injection once the tumors reached approximately 100 mm³. Animals were euthanized when tumor volumes reach 800-1000 mm³. Tumor volume was calculated with the formula (volume=½×length×width×width). Mice showing durable regressions were terminated around day 60.

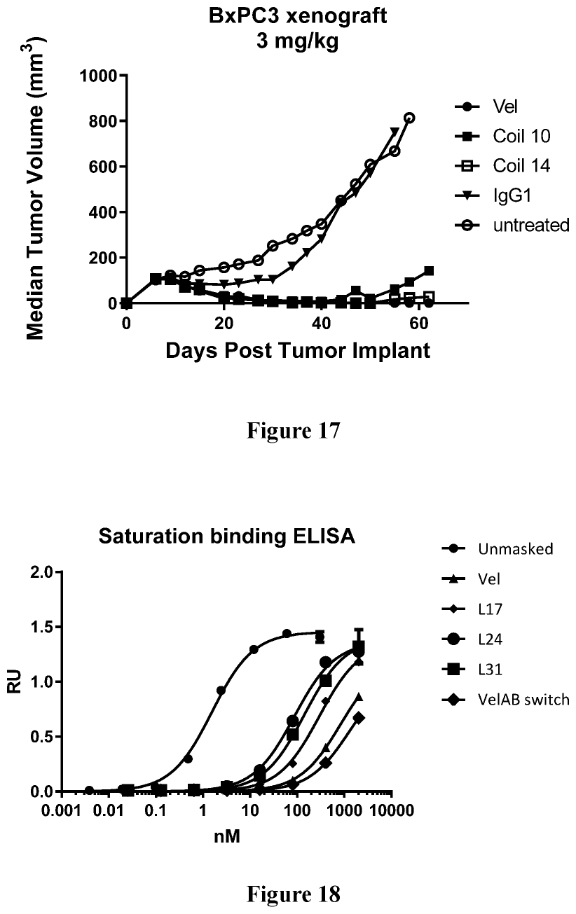

Coil 10 and Coil 14 ADCs had comparable activity to wild-type Vel-IPV-Ab1 antibody in the HPAF-II model (FIG. 16) and BxPC3 model (FIG. 17), as tumor volume was substantially decreased compared to untreated animals or animals treated with IgG1. These data indicate that Vel-IPV-Ab1 ADCs can be successfully demasked in vivo by proteases and have anti-tumor activity.

### Example 12. Analysis of Saturation Binding of Other Vel-Masked Antibodies

The effects of Vel masking of other antibodies was also assessed.

Several variants of Vel masked hB6H12.3 antibodies were evaluated. ELISA assays were run as described in Example 8 using recombinant human CD47. Variants selected for these ELISAs included L17I/L17A (L17), L24I/L24A (L24), L31I/L31A (L31), and VelA/VelB switch as described in Table 8. ELISA results show successful masking of hB6H12.3 antibodies with these different Vel masks (FIG. 18). The hB6H12.3 antibody was also successfully masked with Vel mask variants with heavy chain mutations of L24I/L31I and light chain mutations of L24A/L31A or with heavy chain mutations of D8K and E36H (FIG. 19).

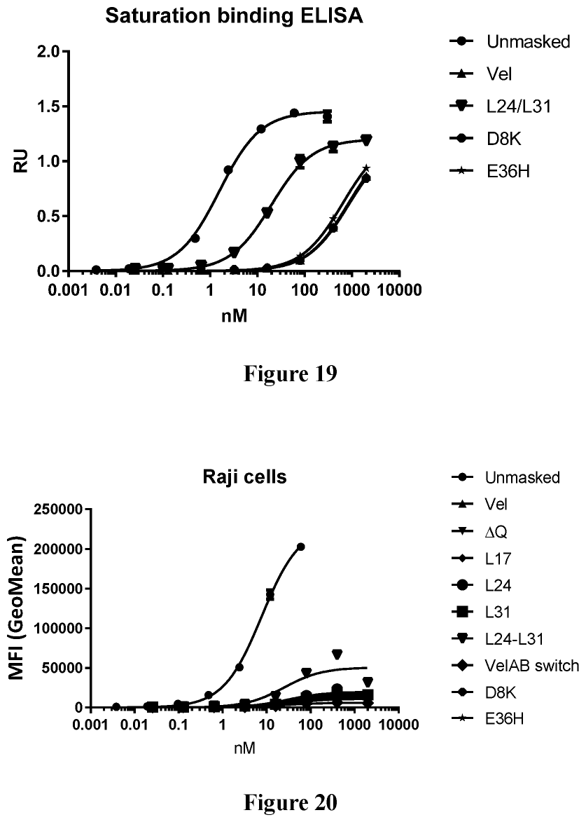

Several variants of Vel masked hB6H12.3 antibodies were evaluated for saturation binding using FACS analysis as described in Example 9 using CD47 (+) Raji cells. All of the Vel masked hB6H12.3 antibodies showed decreased binding compared to the unmasked antibody using FACS analysis (FIG. 20).

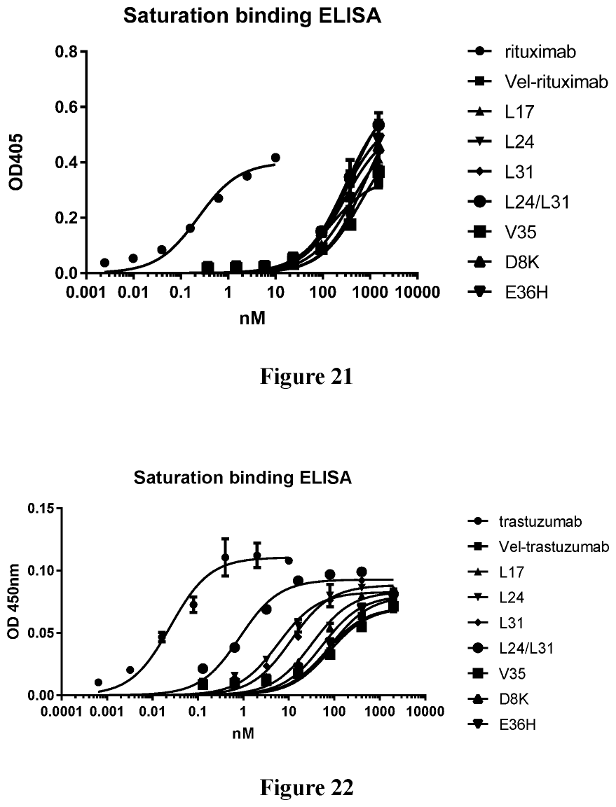

Several variants of Vel masked rituximab antibodies (variants described in Table 10) were also evaluated for saturation binding using ELISA analysis as described in Example 8 using recombinant human CD20. All of the Vel masked rituximab antibodies showed decreased binding compared to the unmasked antibody (FIG. 21).

In addition, several variants of Vel masked trastuzumab antibodies (variants described in Table 9) were evaluated for saturation binding using ELISA analysis as described in Example 8 using recombinant human HER2. All of the Vel masked trastuzumab antibodies showed decreased binding compared to the unmasked antibody (FIG. 22).

These data show that multiple antibodies can be successfully masked with a range of Vel variants.

### Example 13. In Vivo Evaluation of Vel-Masked Anti-CD47 Antibodies

The in vivo activity of masked anti-CD47 antibodies was evaluated in a CD47-positive Detroit562 xenograft model in nude mice. Antibodies were dosed at 1 mg/kg. To conduct the efficacy experiments, 5×10⁶ cells were injected subcutaneously into 5 female nude mice (Harlan) per group. Mice were randomly divided to study groups and dosed with test article via intraperitoneal injection once the tumors reached approximately 100 mm³. Animals were euthanized when tumor volumes reach 800 mm³. Tumor volume was calculated with the formula (volume=½×length×width×width).

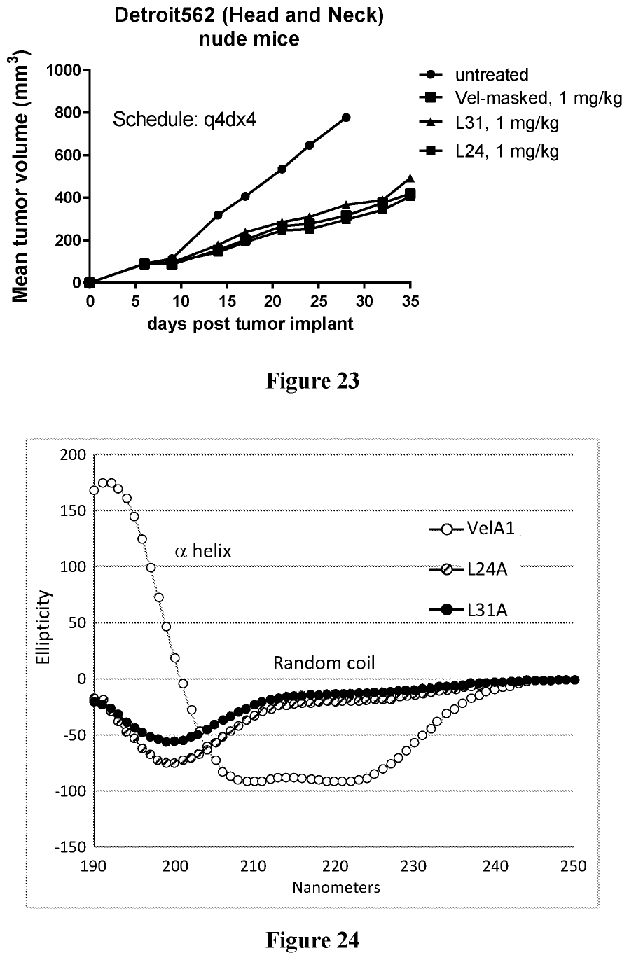

All of the masked anti-CD47 antibodies tested showed in vivo efficacy, as measured by a decrease in tumor growth over time compared to the untreated group (FIG. 23). These data indicate that Vel-masked anti-CD47 antibodies can be demasked in vivo by proteases and have anti-tumor activity.

### Example 14. Circular Dichroism Evaluation of Homodimeric and Heterodimeric Affinity of VelA Variants

Circular dichroism experiments were performed to evaluate homodimerization and heterodimerization of VelA and various mutants.

Circular dichroism experiments were conducted at pH 7.4 to determine the relative affinities of the VelA and VelB in solution. Circular dichroism studies conducted at neutral pH demonstrated that while the VelA (light chain) peptide is alpha-helical under these conditions, the L24A and L31A modifications are not structured, indicating that the VelA wild-type peptide is prone to forming an ordered homodimeric structure at neutral pH (FIG. 24).

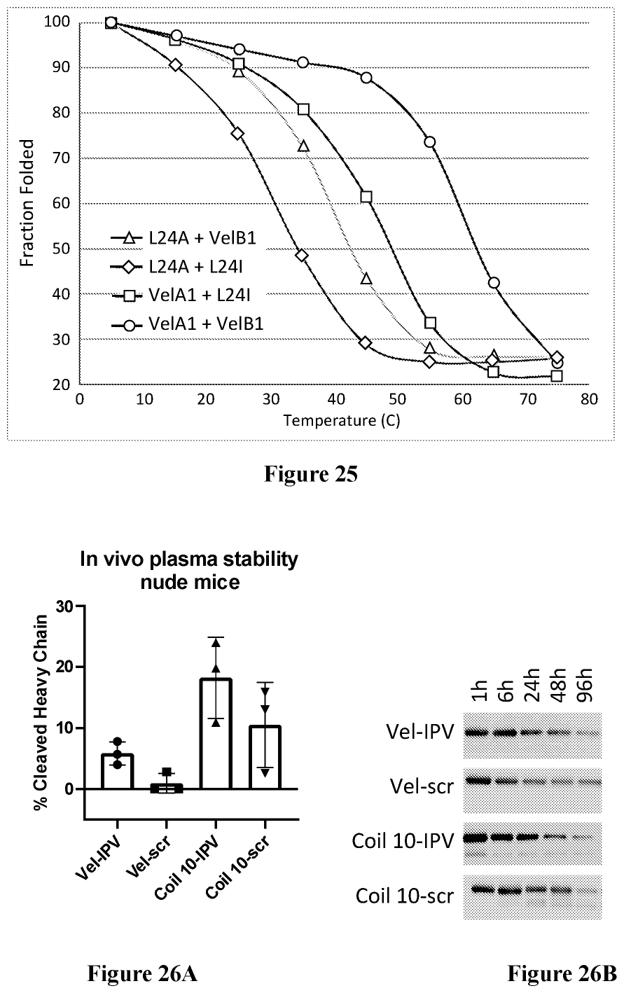

Circular dichroism studies were also performed to determine the relative heterodimeric affinities of the VelA and VelB peptides. Wildtype VelA or VelB is noted as “VelA1” or “VelB1.” The L24A is a VelA variant while the L24I is a VelB variant. The L24A+L24I conditions refer to a L24A VelA variant and a L24I VelB variant. The L24 set of modifications decreased heterodimeric affinity compared to the wild-type VelA+VelB peptides, as demonstrated by a lower melting temperature (FIG. 25). While the affinity is decreased, the heterodimer is still able to form a coiled-coil structure, which results in effective antibody blockade.

Thus, circular dichroism experiments demonstrated that 1) the VelA L24A and L31A mutations decrease VelA homodimerization, as judged by helical content and that 2) the VelA and VelB L24 dual modifications result in moderately decreased heterodimeric affinity.

### Example 15. Cleavage and Stability Analysis of Certain Variants

The effects of MMP-cleavable and MMP-non-cleavable sequences were examined.

In vivo plasma stability was assessed for masked anti-Antigen 1 antibodies using Vel-IPV-Ab1, Vel-IPV-Ab1 variant having mutations L24I/L24A (Coil 10), as well as Vel-scr-Ab1 and the Vel-scr-Ab1 having the Coil 10 mutations to examine the impact of MMP-cleavable (IPV) and MMP-non-cleavable (scr) cleavage sequences. Mask stability was assessed using Western blot after Protein A immunocapture from nude mice dosed with 3 mg/kg of the respective antibody. The extent of cleavage of the heavy chain peptide is shown at 48 hours post-dose (FIG. 26A-B). Cleavage of the Coil 10 mask was observed irrespective of cleavage sequence used. This was hypothesized to be non-specific proteolysis because the proteolysis of the Coil 10 mask increased for both MMP-cleavable and non-cleavable sequences. The increased non-specific proteolysis was hypothesized to be due to the decrease in coil heterodimer affinity that was described in Example 14.

The effects of Vel masking of other antibodies were also assessed by saturation binding experiments.

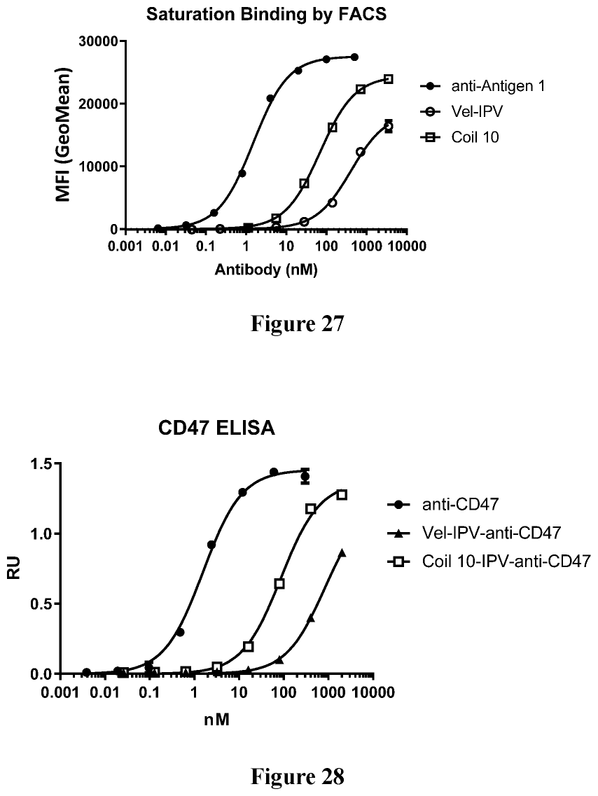

Saturation binding on Ab1 target-expressing HEK293 cells was analyzed for Ab1, Vel-IPV-Ab1, and Vel-IPV-Ab1 Coil 10 antibodies (FIG. 27).

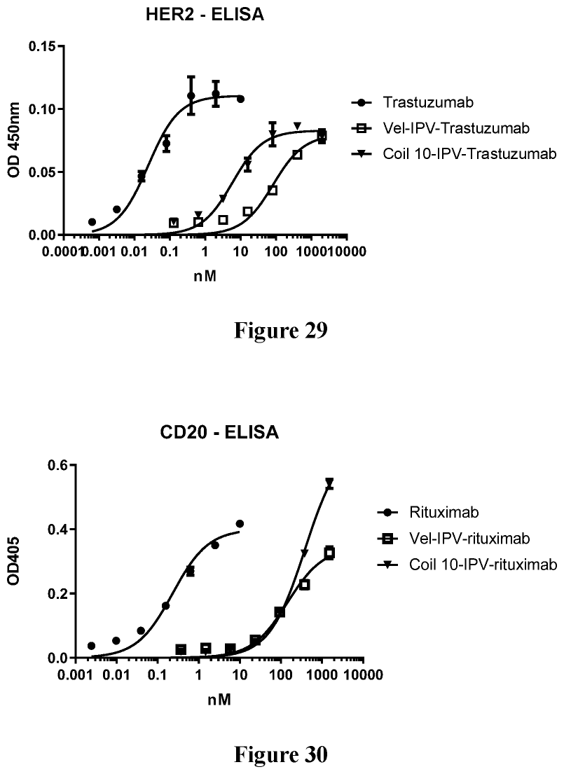

ELISA assays were run as described in Example 8 using recombinant human CD47 to analyze hB6H12.3, Vel-IPV-hB6H12.3, and Vel variant Coil 10 antibodies against recombinant human CD47 (FIG. 28). ELISA assays were also run to analyze trastuzumab, Vel-IPV-trastuzumab, and Vel variant Coil 10 antibodies against recombinant HER2 (FIG. 29). ELISA assays were run to analyze rituximab, Vel-IPV-rituximab, and Vel variant Coil 10 against recombinant human CD20 (FIG. 30).

The Coil 10 antibodies worked well to improve aggregation, but in some cases did not provide as much blockage as the Vel coil.

### Example 16. Additional Mutagenesis Analysis to Identify Coiled-Coil Peptides with Improved Stability

Additional variants were generated to identify Vel coiled-coil masking domains with good aggregation properties and proteolytic stability characteristics.

Single point mutations in either the VelA (light chain) or VelB (heavy chain) sequences were made in Vel-IPV-Ab1.

A series of mutations were explored at the L24 positions of the light and heavy chain coiled-coils on the Ab1 antibody. Antibodies were expressed on a 30 mL scale in Expi-293 cells, Protein A purified, neutralized, and assessed for initial aggregation by analytical size-exclusion chromatography, as described generally in Example 5. To further evaluate the stability of aggregate formation, antibodies were buffer exchanged into PBS, pH 7.4 and concentrated to 10 mg/mL. The extent of aggregation was assessed by analytical SEC immediately thereafter, as well as upon incubation at room temperature for 7 days.

Table 12 presents a summary of aggregation properties by different Vel-IPV-Ab1 antibody variants at position 24.

TABLE 12
Stability characteristics of additional Vel-IPV-Ab1 antibody variants

| | | | % HMW | | |
|---|---|---|---|---|---|
| | | | neutralized | | |
| | | | (Protein A | % HMW | % HMW |
| | | | elution | PBS t = 0 d, | PBS t = 7 d, |
| Coil No. | Heavy chain | Light chain | buffer) | 10.0 mg/mL | 10.0 mg/mL |
| 1 | WT VelB | WT VelA | 15.5 | 19.3 | 65.8 |
| 103 | L24G | L24Y | 1.0 | 1.1 | 1.2 |
| 104 | L24A | L24Y | 0.8 | 0.9 | 1.3 |
| 105 | L24Y | L24W | 10.4 | 19.7 | 21.4 |
| 106 | L24Y | L24F | 3.5 | 4.7 | 5.2 |
| 107 | L24S | L24F | 1.0 | 1.0 | 1.0 |
| 108 | L24Y | L24Y | 8.4 | 19.6 | 20.1 |
| 109 | L24S | L24S | 1.7 | 1.6 | 1.7 |
| 110 | L24G | L24W | 1.5 | 1.5 | 2.2 |
| 111 | L24G | L24F | 2.1 | 1.8 | 2.0 |
| 112 | L24F | L24Y | 4.2 | 7.4 | 8.9 |
| 113 | L24A | L24W | 1.2 | 1.2 | 1.6 |
| 114 | L24W | L24F | 5.3 | 8.9 | n.d. |
| 115 | L24W | L24Y | 12.6 | 25.0 | 26.5 |
| 116 | L24A | L24F | 1.3 | 1.1 | 1.2 |
| 117 | L24F | L24W | 4.5 | 6.7 | 7.9 |
| 118 | L24F | L24S | 1.5 | 1.4 | 2.0 |
| 119 | L24F | L24G | 1.1 | 1.2 | 1.7 |
| 120 | L24F | L24A | 0.6 | 0.7 | 0.7 |
| 121 | L24W | L24A | 0.9 | 1.0 | 1.5 |
| 122 | L24Y | L24A | 0.6 | 0.5 | n.d. |
| 123 | L24W | L24S | 1.3 | 1.1 | 1.9 |
| 124 | L24S | L24W | 0.8 | 1.5 | 1.3 |
| 125 | L24W | L24G | 1.3 | 1.3 | 1.9 |
| 126 | L24Y | L24S | 0.9 | 1.0 | 1.4 |
| 127 | L24Y | L24G | 1.0 | 0.9 | 1.0 |
| 128 | L24F | L24F | 1.8 | 2.1 | 3.4 |
| 129 | L24S | L24Y | 0.8 | 0.9 | 1.3 |
| 10 (L24) | L24I | L24A | 1.2 | 1.4 | n.d. |
| 130 | WT | L24I | 27.7 | n.d. | n.d. |
| 131 | L24I | WT | 36.0 | n.d. | n.d. |
| 132 | L24I | L24V | 1.7 | 1.4 | 1.5 |
| 133 | L24V | L24I | 1.9 | 1.5 | 2.1 |
| 134 | L24K | L24I | 0.9 | 0.3 | 0.3 |
| 135 | L24I | L24K | 1.6 | 1.4 | 9.0 |
| 136 | WT | L24V | 15.7 | 35.8 | 26.5 |
| 137 | L24V | WT | 16.4 | n.d. | n.d. |

(n.d. = not determined)

An additional series of mutations were explored at the V28 positions of the light and heavy chain coiled-coils on the Ab1 antibody to identify other amino acids at this position that could improve stability and reinforce the heterodimer affinity. Antibodies were expressed on a 30 mL scale in Expi-293 cells, Protein A purified, neutralized, and assessed for initial aggregation by analytical size-exclusion chromatography, as described generally above, and further evaluated for stability by buffer exchanging into PBS, pH 7.4. and concentrated to 10 mg/mL. The extent of aggregation was assessed by analytical SEC immediately thereafter and upon incubation at room temperature for 7 days.

Table 13 presents a summary of aggregation properties by different Vel-IPV-Ab1 antibody variants at position 28.

TABLE 13
Stability characteristics of additional Vel-IPV-Ab1 antibody variants

| | | | % HMW | | |
|---|---|---|---|---|---|
| | | | neutralized | % HMW | % HMW |
| | | | ProA elution | PBS t = 0 d, | PBS t = 7 d, |
| Coil No. | Heavy chain | Light chain | buffer, | 10.0 mg/mL | 10.0 mg/mL |
| 138 | V28L | V28I | 47.0 | n.d. | n.d. |
| 139 | V28I | V28L | 61.9 | n.d. | n.d. |
| 140 | V28I | WT | 48.1 | n.d. | n.d. |
| 141 | WT | V28I | 41.7 | n.d. | n.d. |
| 142 | V28K | V28I | 21.4 | n.d. | n.d. |
| 143 | V28I | V28K | 20.5 | n.d. | n.d. |

(n.d. = not determined)

A series of mutations were explored at both the L24 and V28 positions of the light and heavy chain coiled-coils on the Ab1 antibody. Antibodies were expressed on a 30 mL scale in Expi-293 cells, Protein A purified, neutralized, and assessed for initial aggregation by analytical size-exclusion chromatography, as described generally above, and further evaluated for stability by buffer exchanging into PBS, pH 7.4. and concentrated to 10 mg/mL. The extent of aggregation was assessed by analytical SEC immediately thereafter and upon incubation at room temperature for 7 days.

Table 14 presents a summary of aggregation properties by different Vel-IPV-Ab1 antibody variants at position 24 and at position 28.

TABLE 14
Stability characteristics of additional Vel-IPV-Ab1 antibody variants

| | | | % HMW | | |
|---|---|---|---|---|---|
| | | | neutralized | % HMW PBS | % HMW |
| | | | ProA elution | t = 0 d, | PBS t = 7 d, |
| Coil No. | Heavy chain | Light chain | buffer, | 10.0 mg/mL | 10.0 mg/mL |
| 144 | L24I/V28I | L24A | 8.9 | 29.0 | n.d. |
| 145 | V28I | L24A | 34.4 | n.d. | n.d. |
| 146 | L24V/V28I | L24A | 1.5 | 0.8 | 1.3 |
| 147 | L24I | L24A/V28I | 1.7 | 1.5 | 1.3 |
| 148 | L24I/V28L | L24A/V28L | 2.2 | 3.9 | 4.8 |
| 149 | L24I/V28I | L24A | 6.0 | 2.2 | 1.1 |
| 150 | L24I/V28I | L24A/V28I | 4.8 | 9.7 | 8.3 |
| 151 | V28L | V28L | 51.5 | n.d. | n.d. |

(n.d. = not determined)

Under these conditions, certain mutations at position 24 in both the light and heavy chain showed improved aggregation properties.

### Example 17. Analysis of ELISA Binding of Additional Vel-IPV-Ab1 Variants

The impact of Vel coiled-coil mutations on binding was assessed by saturation binding to recombinant antigen by ELISA, as described in Example 8.

Table 15 presents saturation binding (EC50 in nM and Bmax in OD at 450 nm) and calculates the EC50-fold change from unmasked Ab1 for each of the variants.

TABLE 15
ELISA saturation binding of variants against recombinant Antigen 1

| | | | | Binding | EC50 fold |
|---|---|---|---|---|---|
| | | | Binding | Bmax | change from |
| Coil No. | Heavy chain | Light chain | EC50 (nM) | (OD450) | Ab1 |
| | Unmasked Ab1 | Unmasked Ab1 | 0.29 | 1.8 | NA |
| 1 | WT VelB | WT VelA | 138.9 | 1.3 | 479 |
| 103 | L24G | L24Y | 75.3 | 0.6 | 260 |
| 104 | L24A | L24Y | 121.5 | 0.5 | 419 |
| 105 | L24Y | L24W | 199.5 | 1.1 | 688 |
| 106 | L24Y | L24F | 190.5 | 0.8 | 657 |
| 107 | L24S | L24F | 132.7 | 0.6 | 458 |
| 108 | L24Y | L24Y | 145.8 | 1.0 | 503 |
| 109 | L24S | L24S | 229.8 | 0.8 | 792 |
| 110 | L24G | L24W | 548.5 | 1.0 | 1891 |
| 111 | L24G | L24F | 619.2 | 1.3 | 2135 |
| 112 | L24F | L24Y | 217.7 | 0.8 | 751 |
| 113 | L24A | L24W | 198.7 | 0.6 | 685 |
| 114 | L24W | L24F | 278.1 | 1.1 | 959 |
| 115 | L24W | L24Y | 183.7 | 1.0 | 633 |
| 116 | L24A | L24F | 368.1 | 0.7 | 1269 |
| 117 | L24F | L24W | 322.5 | 1.1 | 1112 |
| | Unmasked Ab1 | Unmasked Ab1 | 0.25 | 2.0 | NA |
| 118 | L24F | L24S | 45.2 | 0.8 | 181 |
| 119 | L24F | L24G | 70.0 | 0.9 | 280 |
| 120 | L24F | L24A | 102.1 | 0.7 | 408 |
| 121 | L24W | L24A | 140.4 | 0.9 | 562 |
| 122 | L24Y | L24A | 85.9 | 0.6 | 344 |
| 123 | L24W | L24S | 275.2 | 0.9 | 1101 |
| 124 | L24S | L24W | 129.3 | 0.6 | 517 |
| 125 | L24W | L24G | 183.8 | 1.1 | 735 |
| 126 | L24Y | L24S | 110.9 | 0.7 | 444 |
| 127 | L24Y | L24G | 161.5 | 0.7 | 646 |
| 128 | L24F | L24F | 122.2 | 0.9 | 489 |
| 129 | L24S | L24Y | 76.6 | 0.6 | 306 |
| | Unmasked Ab1 | Unmasked Ab1 | 0.22 | 2.2 | NA |
| 1 | WT VelB | WT VelA | 98.1 | 1.9 | 446 |
| 10 (L24) | L24I | L24A | 28.0 | 1.0 | 127 |
| 130 | WT | L24I | 144.6 | 1.4 | 657 |
| 131 | L24I | WT | 211.2 | 1.3 | 960 |
| 132 | L24I | L24V | 48.2 | 0.8 | 219 |
| 133 | L24V | L24I | 83.5 | 0.8 | 380 |
| 134 | L24K | L24I | 14.7 | 0.7 | 67 |
| 135 | L24I | L24K | 9.4 | 1.0 | 43 |
| 136 | L24L | L24V | 75.9 | 1.1 | 345 |
| 137 | L24V | WT | 128.4 | 1.2 | 584 |
| 138 | V28L | V28I | 117.1 | 1.8 | 532 |
| 139 | V28I | V28L | 113.5 | 1.7 | 516 |
| 140 | V28I | WT | 79.1 | 1.7 | 360 |
| 141 | WT | V28I | 113.1 | 1.8 | 514 |
| 142 | V28K | V28I | 101.3 | 1.8 | 461 |
| | Unmasked Ab1 | Unmasked Ab1 | 0.24 | 2.0 | NA |
| 143 | V28I | V28K | 96.6 | 1.5 | 403 |
| 144 | L24I/V28I | L24A | 14.3 | 1.2 | 60 |
| 145 | V28I | L24A | 69.6 | 1.2 | 290 |
| 146 | L24V/V28I | L24A | 30.7 | 1.0 | 128 |
| 147 | L24I | L24A/V28I | 89.3 | 1.0 | 372 |
| 148 | L24I/V28L | L24A/V28L | 82.2 | 1.1 | 343 |
| 149 | L24I/V28I | L24A | 43.6 | 1.1 | 182 |
| 150 | L24I/V28I | L24A/V28I | 35.9 | 1.2 | 150 |
| 151 | V28L | V28L | 198.4 | 1.5 | 827 |

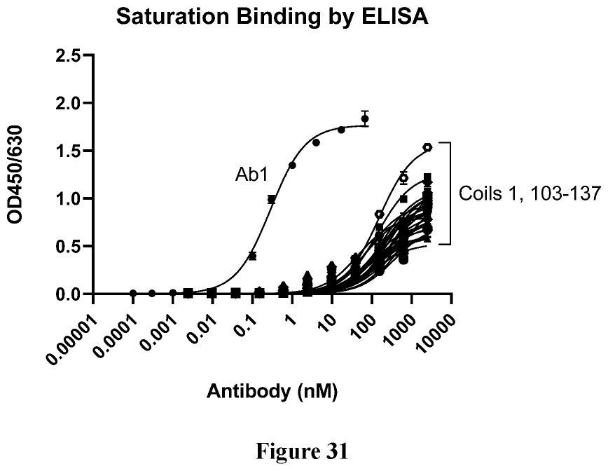

This is also shown in FIG. 31, which depicts saturation binding ELISA for Ab1, Vel-IPV-Ab1, and Vel-IPV-Ab1 variants (Coils 103-137).

### Example 18. Analysis of FACS Binding of Additional Vel-IPV-Ab1 Variants

Select Vel-IPV-Ab1 variants were also assessed by FACS analysis, as described generally in Example 9.

Table 16 presents saturation binding determined by flow cytometry (EC50 in nM) for Ab1, Vel-IPV-Ab1, and selected Vel-IPV-Ab1 variants, and calculates the EC50-fold change from unmasked Ab1 for each of the variants.

TABLE 16
FACS saturation binding of variants against
Antigen 1-expressing HEK293 cells antigen

| | | | Binding | EC50 fold |
|---|---|---|---|---|
| | | | EC50 | change from |
| Coil No. | Heavy chain | Light chain | (nM) | Ab1 |
| | Unmasked Ab1 | Unmasked Ab1 | 0.14 | NA |
| 1 | WT VelB | WT VelA | 93.0 | 703 |
| 103 | L24G | L24Y | 2.1 | 15 |
| 104 | L24A | L24Y | 9.6 | 69 |
| 105 | L24Y | L24W | 73.4 | 524 |
| 106 | L24Y | L24F | 38.4 | 274 |
| 107 | L24S | L24F | 2.8 | 20 |
| 108 | L24Y | L24Y | 77.5 | 554 |
| 109 | L24S | L24S | 4.7 | 34 |
| 110 | L24G | L24W | 42.9 | 306 |
| 111 | L24G | L24F | 9.1 | 65 |
| 112 | L24F | L24Y | 107.0 | 750 |
| 113 | L24A | L24W | 35.0 | 250 |
| 114 | L24W | L24F | 59.3 | 423 |
| 115 | L24W | L24Y | 152.3 | 1088 |
| 116 | L24A | L24F | 7.5 | 54 |
| 117 | L24F | L24W | 144.3 | 1031 |
| 118 | L24F | L24S | 16.8 | 120 |
| 119 | L24F | L24G | 139.2 | 994 |
| 120 | L24F | L24A | 49.3 | 352 |
| 121 | L24W | L24A | 132.0 | 943 |
| 122 | L24Y | L24A | 14.5 | 104 |
| 123 | L24W | L24S | 10.0 | 71 |
| 124 | L24S | L24W | 14.1 | 10 |
| 125 | L24W | L24G | 14.0 | 100 |
| 126 | L24Y | L24S | 9.5 | 68 |
| 127 | L24Y | L24G | 7.8 | 56 |
| 128 | L24F | L24F | 45.4 | 324 |
| 129 | L24S | L24Y | 2.6 | 19 |
| 10 (L24) | L24I | L24A | 51.1 | 365 |
| 132 | L24I | L24V | 29.0 | 207 |
| 133 | L24V | L24I | 33.7 | 241 |
| 136 | L24L | L24V | 83.3 | 595 |
| 147 | L24I | L24A/V28I | 37.3 | 266 |
| 148 | L24I/V28L | L24A/V28L | 70.1 | 501 |

### Example 19. In Vivo Stability Screen of Vel-IPV-Ab1 Variants

The in vivo plasma stability of selected Vel-IPV-Ab1 variants was assessed using Western blot analysis. A subset of variants, with mutations at the L24 and V28 sites in light and/heavy chain peptides, were dosed intravenously to nude mice. At 24- and 48-hours post-dose, plasma samples were collected, human antibodies were purified using Protein A resin and separated using SDS-PAGE, and the integrity of the masked Abs was assessed by Western blotting using an anti-human Fc-HRP antibody. Data from two mice per group was used at each timepoint.

Table 17 presents stability of Vel-IPV-Ab1 and selected Vel-IPV-Ab1 variants in vivo, as assessed by Western blot analysis of cleaved heavy chain after 24 or 48 hours.

TABLE 17
Stability of variants.

| | | | % Cleaved Heavy Chain | | | |
|---|---|---|---|---|---|---|
| Antibody Variant | | | 24 hours | 24 hours | 72 hours | 72 hours |
| Coil | Heavy | Light | Mean | Std Dev | Mean | Std Dev |
| No. | chain | chain | (%) | (%) | (%) | (%) |
| 1 | WT VelB | WT VelA | 6.6 | 4.8 | 8.8 | 6.8 |
| 10 (L24) | L24I | L24A | 22.6 | 10.3 | 30.1 | 2.6 |
| 106 | L24Y | L24F | 9.6 | 1.4 | 16.9 | 3.9 |
| 110 | L24G | L24W | 16.8 | 1.3 | 29.9 | 2.8 |
| 112 | L24F | L24Y | 8.7 | 1.3 | 12.2 | 0.6 |
| 113 | L24A | L24W | 16.6 | 3.8 | 22.2 | 4.7 |
| 114 | L24W | L24F | 11.7 | 2.2 | 15.7 | 4.7 |
| 116 | L24A | L24F | 10.5 | 2.3 | 18.6 | 5.2 |
| 117 | L24F | L24W | 3.7 | 0.6 | 4.5 | 1.6 |
| 118 | L24F | L24S | 18.1 | 10.2 | 41.6 | 29.1 |
| 120 | L24F | L24A | 11.0 | 5.7 | 12.3 | 3.6 |
| 121 | L24W | L24A | 10.5 | 0.3 | 12.3 | 3.6 |
| 122 | L24Y | L24A | 22.9 | 9.9 | 38.1 | 3.2 |
| 123 | L24W | L24S | 16.8 | 1.2 | 33.1 | 18.6 |
| 124 | L24S | L24W | 15.7 | 11.9 | 17.7 | 8.7 |
| 125 | L24W | L24G | 6.5 | 1.6 | 13.9 | 0.8 |
| 126 | L24Y | L24S | 24.5 | 14.6 | 31.0 | 37.4 |
| 127 | L24Y | L24G | 26.5 | 18.6 | 36.9 | 27.8 |
| 128 | L24F | L24F | 14.2 | 3.7 | 22.3 | 9.9 |
| 130 | WT | L24I | 7.0 | 7.8 | 6.5 | 4.9 |
| 131 | L24I | WT | 3.6 | 1.2 | 3.3 | 0.6 |
| 132 | L24I | L24V | 6.0 | 0.4 | 14.5 | 9.5 |
| 133 | L24V | L24I | 7.7 | 7.8 | 15.2 | 13.9 |
| 146 | L24V/V28I | L24A | 4.2 | 1.3 | 5.2 | 0.0 |
| 147 | L24I | L24A/V28I | 9.6 | 8.6 | 10.2 | 7.7 |
| 148 | L24I/V28L | L24A/V28L | 5.0 | 0.6 | 8.3 | 1.2 |
| 149 | L24I/V28I | L24A | 2.0 | 0.6 | 3.6 | 0.4 |
| 150 | L24I/V28I | L24A/V28I | 2.6 | 3.6 | 15.1 | 8.0 |

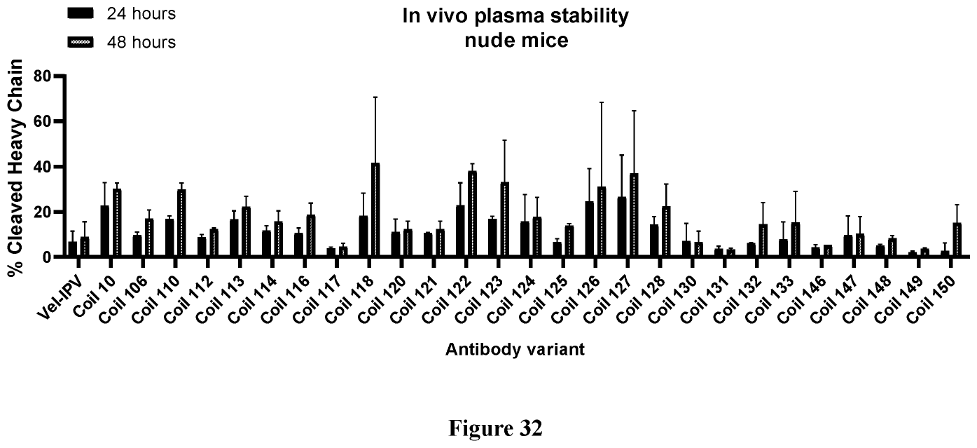

This is also shown in FIG. 32, which graphs selected variants versus % cleaved heavy chain at 24- and 48-hours.

These data show that the Coil 10 mutations (L24I/L24A), combined with mutations at position 28 designed to reinforce the heterodimer affinity, resulted in variants, such as Coils 147 and 148, showing improved stability compared to Coil 10.

### Example 20. Analysis of Stability and Saturation Binding of Other Vel-Masked Antibodies

The effects of Vel masking by selected variants of other antibodies was also assessed.

Selected Vel-masked variants were applied to masking of anti-CD47 antibody hB6H12.3 and trastuzumab targeting HER2. Antibodies were expressed on a 30 mL scale in Expi-293 cells, Protein A purified, neutralized, and assessed for initial aggregation by analytical size-exclusion chromatography. To evaluate the stability of aggregate formation, antibodies were buffer exchanged into PBS, pH 7.4. and concentrated to 10 mg/mL. The extent of aggregation was assessed by analytical SEC immediately thereafter and upon incubation at room temperature for 7 days. These results are presented in Tables 18-19.

TABLE 18
Stability characteristics of selected coiled-coil mutations
on Vel-IPV-hB6H12.3 expressed in Expi-293.

| | | | % HMW | % HMW PBS | % HMW |
|---|---|---|---|---|---|
| | | | neutralized ProA | t = 0 d, | PBS t = 7 d, |
| Coil No. | Heavy chain | Light chain | elution buffer, | 10.0 mg/mL | 10.0 mg/mL |
| 146 | L24V/V28I | L24A | 7.2 | 7.5 | 7.0 |
| 147 | L24I | L24A/V28I | 3.1 | 4.1 | 4.8 |
| 148 | L24I/V28L | L24A/V28L | 7.7 | 8.7 | 8.7 |
| 149 | L24I/V28I | L24A | 10.9 | 22.6 | 22.6 |
| 117 | L24F | L24W | 6.8 | 13.6 | 15.4 |

(n.d. = not determined)

TABLE 19
Stability characteristics of selected coiled-coil mutations
on Vel-IPV-trastuzumab expressed in Expi-293.

| | | | % HMW | % HMW PBS | % HMW |
|---|---|---|---|---|---|
| | | | neutralized ProA | t = 0 d, | PBS t = 7 d, |
| Coil No. | Heavy chain | Light chain | elution buffer, | 10.0 mg/mL | 10.0 mg/mL |
| 146 | L24V/V28I | L24A | 2.6 | 3.1 | 2.8 |
| 147 | L24I | L24A/V28I | 2.6 | 3.2 | 2.8 |
| 148 | L24I/V28L | L24A/V28L | 2.8 | 3.2 | 2.9 |
| 149 | L24I/V28I | L24A | 4.5 | 5.3 | 5.6 |
| 117 | L24F | L24W | 3.3 | 4.0 | 3.9 |

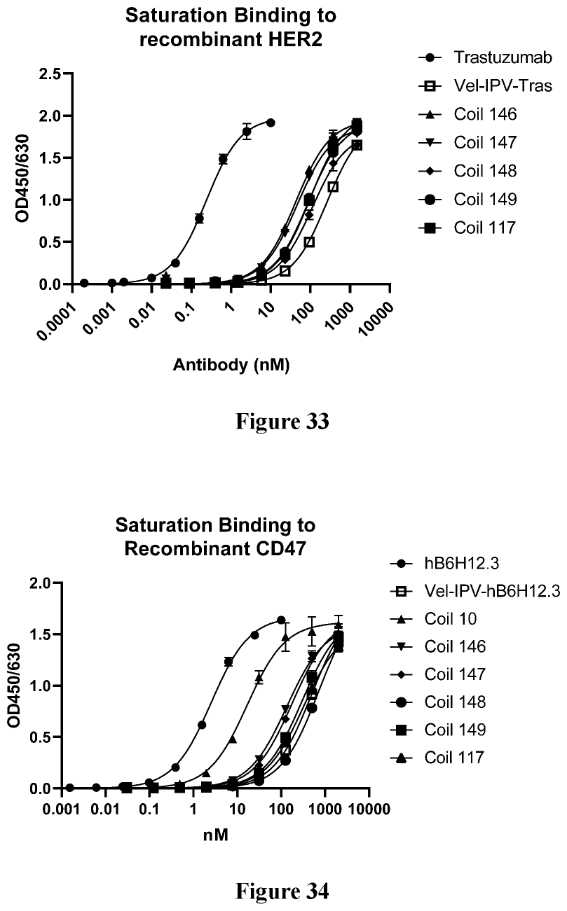

Selected Vel-masked trastuzumab antibody variants (variants of Table 12 and 14) were also evaluated for saturation binding using ELISA analysis as described in Example 8 and 11 using recombinant human HER2. These results are presented in Table 20, as well as in FIG. 33.

TABLE 20
ELISA saturation binding for trastuzumab, Vel-IPV-trastuzumab and
Vel-IPV-trastuzumab variants against recombinant HER2 antigen.

| | | | | Binding | EC50 fold |
|---|---|---|---|---|---|
| | | | Binding | Bmax | change from |
| Coil No. | Heavy chain | Light chain | EC50 (nM) | (OD450) | Trastuzumab |
| | Unmasked | Unmasked | 0.24 | 2.0 | NA |
| | trastuzumab | trastuzumab | | | |
| 1 | WT VelB | WT VelA | 261.9 | 1.9 | 1091 |
| 146 | L24V/V28I | L24A | 42.9 | 2.0 | 179 |
| 147 | L24I | L24A/V28I | 46.4 | 1.9 | 193 |
| 148 | L24I/V28L | L24A/V28L | 110.2 | 1.8 | 459 |
| 149 | L24I/V28I | L24A | 88.0 | 1.9 | 367 |
| 117 | L24F | L24W | 98.8 | 2.0 | 412 |

Vel-masked hB6H12.3 antibody variants were also evaluated for saturation binding. ELISA assays were run as described in Examples 8 and 12 using recombinant human CD47. These results are presented in Table 21 (FIG. 34).

TABLE 21
ELISA saturation binding for hB6H12.3, Vel-IPV-hB6H12.3 and
Vel-IPV-hB6H12.3 variants against recombinant CD47 antigen.

| | | | Binding | Binding | EC50 fold |
|---|---|---|---|---|---|
| Coil | Heavy | Light | EC50 | Bmax | change from |
| No. | chain | chain | (nM) | (OD450) | hB6H12.3 |
| | Unmasked | Unmasked | 2.55 | 1.7 | NA |
| | hB6H12.3 | hB6H12.3 | | | |
| 1 | WT VelB | WT VelA | 494.2 | 1.8 | 194 |
| 10 | L24I | L24A | 16.6 | 1.6 | 6.5 |
| 146 | L24V/V28I | L24A | 133.9 | 1.6 | 53 |
| 147 | L24I | L24A/V28I | 178.3 | 1.7 | 70 |
| 148 | L24I/V28L | L24A/V28L | 719.8 | 1.9 | 282 |
| 149 | L24I/V28I | L24A | 298.0 | 1.7 | 117 |
| 117 | L24F | L24W | 324.6 | 1.6 | 127 |

These data show that multiple antibodies can be successfully masked with a range of Vel variants.

### Example 21. In Vivo Stability Screen of Vel-IPV-hB6H12.3 and Vel-IPV-Trastuzumab Variants

The in vivo plasma stability of selected Vel-IPV-hB6H12.3 and Vel-IPV-trastuzumab variants was assessed using Western blot analysis. A subset of variants, as described in Table 20 (trastuzumab) and Table 21 (hB6H12.3), were dosed intravenously to nude mice, as described above in Example 19. At 24- and 48-hours post-dose, plasma samples were collected.

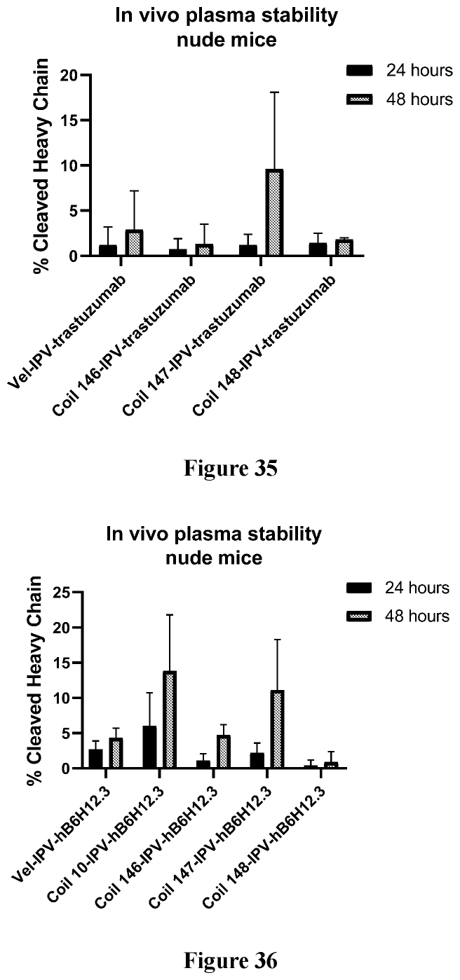

Graphs of selected variants versus % cleaved heavy chain at 24- and 48-hours is shown for trastuzumab (FIG. 35) and hB6H12.3 (FIG. 36).

### Example 22. Additional Mutagenesis Analysis to Identify Coiled-Coil Peptides with Improved Stability

Additional variants were generated to identify Vel coiled-coil masking domains with good aggregation properties and proteolytic stability characteristics.

Single and double point mutations in either the VelA (light chain) or VelB (heavy chain) sequences were made in Vel-IPV-Ab1.

Antibodies were expressed in Expi-293 cells, Protein A purified, neutralized, and assessed for initial aggregation by analytical size-exclusion chromatography, as described generally in Example 5. To further evaluate the stability of aggregate formation, antibodies were buffer exchanged into PBS, pH 7.4 and concentrated to 10 mg/mL. The extent of aggregation was assessed by analytical SEC immediately thereafter, as well as upon incubation at room temperature for 7 days.

Table 22 presents a summary of aggregation properties by different Vel-IPV-Ab1 antibody variants.

TABLE 22
Stability characteristics of additional Vel-IPV-Ab1 antibody variants

| | | | % HMW | % HMW PBS | % HMW |
|---|---|---|---|---|---|
| Coil | Heavy | Light | neutralized ProA | t = 0 d, | PBS t = 7 d, |
| No. | chain | chain | elution buffer, | 10.0 mg/mL | 10.0 mg/mL |
| 164 | L24S/L31Q | L24R/L31V | 1.2 | 1.6 | 1.2 |
| 152 | L24H | V28A/L31E | 2.1 | 2.4 | 2.4 |
| 153 | WT | L24R | 8.2 | 27.1 | 28.8 |
| 154 | V28L | L24K/L31V | 2.6 | 3.6 | 4.7 |
| 155 | V28S/L31W | V28S | 2.1 | 2.7 | 2.7 |
| 156 | L31P | L31T | 2.3 | 2.2 | 1.8 |

These data indicate that a variety of mutations to specific amino acids in VelA and/or VelB can provide beneficial stability characteristics.

### Example 23. Analysis of Stability and Saturation Binding of Other Vel-Masked Antibodies

The effects of Vel masking by selected variants on other antibodies was also assessed.

Selected Vel variants were applied to masking of anti-CD47 antibody hB6H12.3. Antibodies were expressed generally as described above. To evaluate the stability of aggregate formation, antibodies were buffer exchanged into PBS, pH 7.4 and concentrated to 10 mg/mL. The extent of aggregation was assessed by analytical SEC immediately thereafter and upon incubation at room temperature for 7 days. These results are presented in Table 23.

TABLE 23
Stability characteristics of selected coiled-coil mutations
on Vel-IPV-hB6H12.3 expressed in Expi-293.

| | | | % HMW | % HMW PBS | % HMW |
|---|---|---|---|---|---|
| Coil | Heavy | Light | neutralized ProA | t = 0d, | PBS t = 7 d, |
| No. | chain | chain | elution buffer, | 10.0 mg/mL | 10.0 mg/mL |
| 164 | L24S/L31Q | L24R/L31V | 4.2 | 4.9 | 4.7 |
| 152 | L24H | V28A/L31E | 5.5 | 5.9 | 5.5 |
| 153 | WT | L24R | 11.5 | 25.9 | 28.0 |
| 154 | V28L | L24K/L31V | 8.0 | 8.0 | 8.9 |
| 155 | V28S/L31W | V28S | 2.1 | 3.6 | 3.4 |
| 156 | L31P | L31T | 4.0 | 4.7 | 4.4 |

(n.d. = not determined)

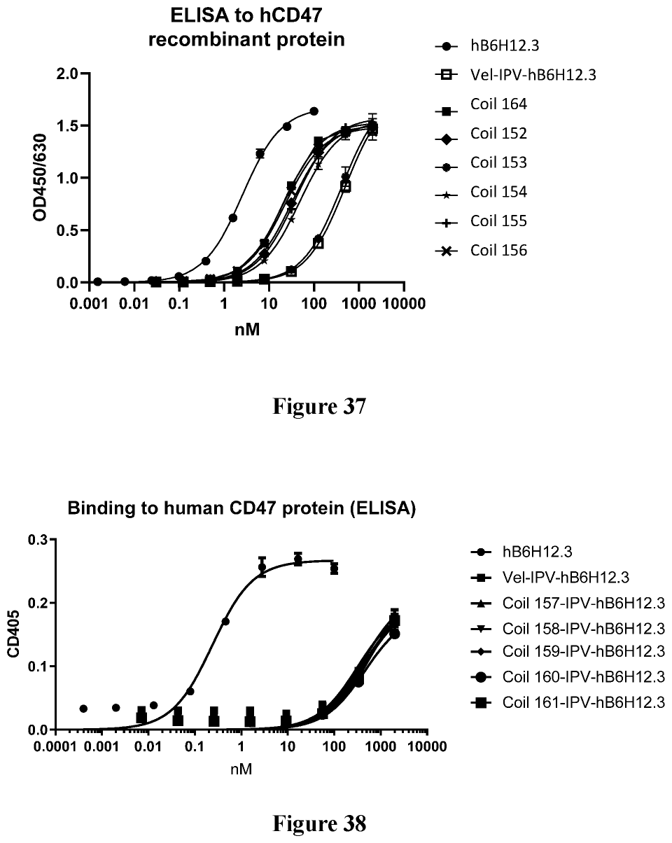

Selected Vel-masked hB6H12.3 antibody variants were also evaluated for saturation binding. ELISA assays were run as described in Examples 8 and 12 using recombinant human CD47. These results are presented in Table 24 (FIG. 37).

TABLE 24
ELISA saturation binding for hB6H12.3, Vel-IPV-hB6H12.3 and
Vel-IPV-hB6H12.3 variants against recombinant CD47 antigen.

| | | | Binding | Binding | EC50 fold |
|---|---|---|---|---|---|
| Coil | Heavy | Light | EC50 | Bmax | change from |
| No. | chain | chain | (nM) | (OD450) | hB6H12.3 |
| | Unmasked | Unmasked | 2.55 | 1.7 | NA |
| | hB6H12.3 | hB6H12.3 | | | |
| 164 | L24S/L31Q | L24R/L31V | 21.5 | 1.5 | 8 |
| 152 | L24H | V28A/L31E | 31.7 | 1.5 | 12 |
| 153 | WT | L24R | 410.0 | 1.8 | 161 |
| 154 | V28L | L24K/L31V | 47.9 | 1.5 | 19 |
| 155 | V28S/L31W | V28S | 37.8 | 1.6 | 15 |
| 156 | L31P | L31T | 22.4 | 1.5 | 9 |

These data show that multiple antibodies can be successfully masked with a range of Vel variants.

### Example 24. Mutagenesis to Impact Glycation of Vel-Masked Antibodies

The Vel mask was found in some instances to have complex O-linked glycation on the light chain.

Additional mutations in the VelA (light chain) were explored to prevent complex this O-linked glycation. Vel masked hB6H12.3 antibody variants were expressed as described above.

Table 25 presents O-linked glycan status for Vel-IPV-hB6H12.3 antibody variants expressed in Expi-293 cells. Mutation or removal of the -TT-dipeptide motif was demonstrated to mitigate O-linked glycation of the light chain coil peptide.

TABLE 25
Core O-glycation analysis of Vel-IPV-
hB6H12.3 antibody variants

| | | N terminal | |
|---|---|---|---|
| | | residues of | |
| | | light chain | |
| | | sequence | |
| | | (VA correspond | |
| | | to positions | |
| | | 1-2 of SEQ | |
| | | ID NQ: 1/ | |
| | | positions 7- | |
| Coil | Mask | 8 of SEQ | Core O- |
| No. | name | ID NQ: 3) | glycosylation |
| 1 | Vel | QGASTTVA | Yes |
| | | (SEQ ID NQ: 192) | |
| 157 | T5A | QGASATVA | No |
| | | (SEQ ID NQ: 193) | |
| 158 | DT5/DT6 | QGASVA | No |
| | | (SEQ ID NQ: 194) | |
| 159 | T6A | QGASTAVA | No |
| | | (SEQ ID NQ: 195) | |
| 160 | S4A | QGAATTVA | Yes |
| | | (SEQ ID NQ: 196) | |
| 161 | T6S | QGASTSVA | No |
| | | (SEQ ID NQ: 197) | |

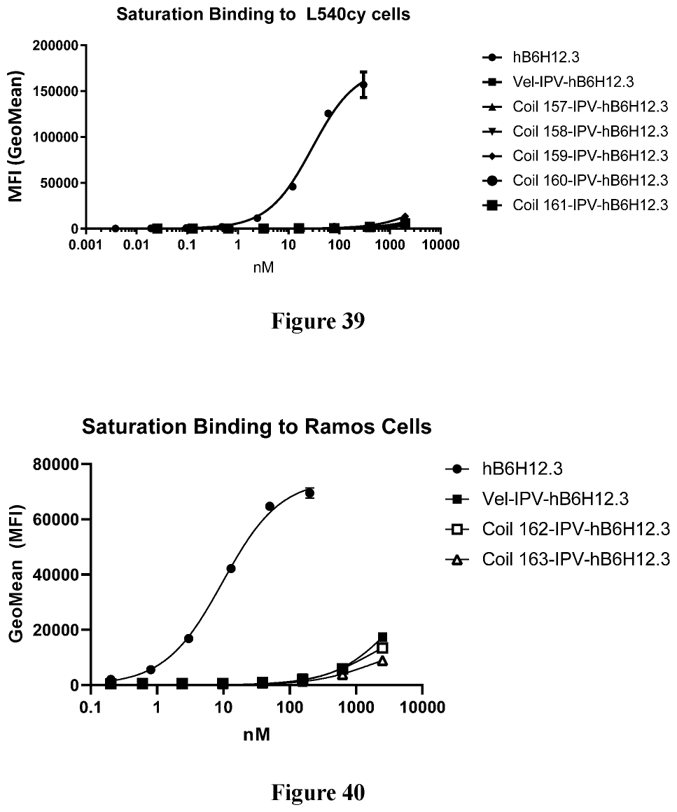

Vel masked hB6H12.3 antibody variants were also evaluated for saturation binding. ELISA assays were run as described in Examples 8 and 12 using recombinant human CD47 (FIG. 38). Saturation binding flow cytometry analysis of these O-glycosylation variants was also assessed for hB6H12.3, Vel-IPV-hB6H12.3 and the above Vel variants against L540cy cells (Cancer Res, Jul. 1, 2002 (62) (13)3736-3742) that express human CD47 antigen (FIG. 39).

The O-glycation variants were further combined with single and double point mutations in either the VelA (light chain) or VelB (heavy chain) sequences in Vel-IPV-hB6H12.3 expressed in CHO-DG44 cells as described in Example 1.

Table 26 presents a summary of aggregation properties by different Vel-IPV-hB6H12.3 antibody variants.

TABLE 26
Stability characteristics of additional Vel-IPV-hB6H12.3 variants

| Coil | Heavy | Light | % HMW neutralized |
|---|---|---|---|
| No. | chain | chain | ProA elution buffer, |
| 1 | WT VelB | WT VelA | 24.3 |
| 146 | L24V/V28I | L24A | 15.9 |
| 148 | L24I/V28L | L24A/V28L | 5.5 |
| 162 | L24V/V28I | T6S/L24A | 5.0 |
| 163 | L24I/V28L | T6S/L24A/V28L | 4.6 |

Vel masked hB6H12.3 antibody variants were also evaluated for saturation binding. Saturation binding flow cytometry analysis for hB6H12.3, Vel-IPV-hB6H12.3 and Vel-IPV-hB6H12.3 variants was assessed against Ramos cells that express human CD47 antigen (FIG. 40) obtained from ATCC. These data demonstrate the three Vel-masked antibodies display a similar profile, suggesting that the mutations in the coiled-coil domains of Coil 162 and Coil 163 does not affect the masking ability compared to wild-type Vel.

### Example 25. Intravenous Single Dose Studies

Variants were assessed in intravenous single dose studies in cynomolgus macaques to evaluate their ability to improve the tolerability of hB6H12.3 in a similar fashion as the Vel.

IV single dose studies were conducted in cynomolgus macaques. The anti-CD47 IgG1 antibodies were cross-reactive with human and cyno CD47 that is highly conserved across these species in expression and sequence. Cynomolgus macaques also have highly similar FcgR interactions with IgG1 antibodies and are considered toxicologically predictive of effector-function related effects of human IgG1 antibodies, making them a suitable model for evaluating the effects of IgG1 anti-CD47 antibodies.

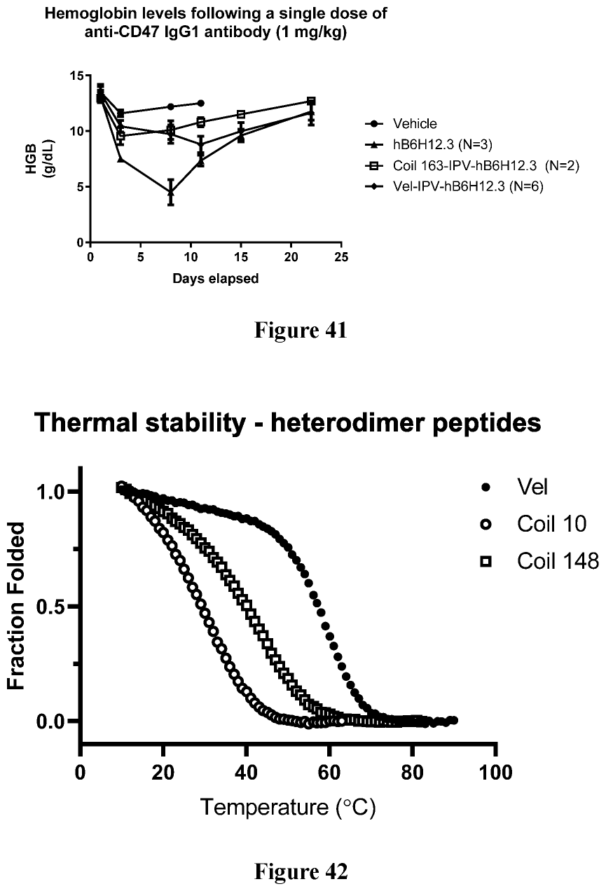

Antibodies were dosed intravenously with 1 mg/kg of each antibody. Treatment with the unmasked hB6H12.3 antibody results in a pronounced decrease in red cell mass by hematology analysis, as demonstrated by a drop in hemoglobin (HGB). Both the Vel- and Coil 163-masked antibodies displayed a similar, marked reduction in the HGB decrease compared to unmasked antibody (FIG. 41).

### Example 26. Analysis of Aggregation of Other Vel-Masked Antibodies

The effects of masking by selected Vel variants on other antibodies was also assessed.

Antibodies previously demonstrated to have challenging aggregation profiles with the Vel mask were produced in Expi-293 cells and the extent of aggregation with various masks was determined post-Protein A purification.

Coil 162 and 163 antibodies were demonstrated to have significant improvements in aggregation compared to the Vel coiled coil. This data is presented in Table 27, which shows the impact of mutations on several antibodies expressed in Expi-293.

TABLE 27
Stability characteristics of Vel-IPV-masked antibodies

| | | | % HMW neutralized ProA elution buffer | | | | |
|---|---|---|---|---|---|---|---|
| Coil | Heavy | Light | Antibody | Antibody | Antibody | Antibody | Antibody |
| No. | chain | chain | 1 | 2 | 3 | 4 | 5 |
| 1 | WT VelB | WT VelA | 63.5 | 70.0 | 48.0 | 23.7 | 20.0 |
| 162 | L24I | T6S/L24A/ | <1 | <1 | <1 | <1 | <1 |
| | | V28I | | | | | |
| 163 | L24I/V28L | T6S/L24A/ | <1 | <1 | <1 | <1 | <1 |
| | | V28L | | | | | |

### Example 27. Circular Dichroism Evaluation of Homodimeric or Heterodimeric Affinity of Vel Variants

Circular dichroism experiments were performed to evaluate homodimerization or heterodimerization affinities of Vel and various mutants.

CD studies were performed with a Jasco J-810 spectropolarimeter. Spectra were measured using a total peptide concentration of 150 μM (PBS, pH 7.4) in a 1 mm cuvette. Thermal denaturation curves were measured at 222 nm from 10° C. to 90° C. in steps of 1° C. The fraction folded (F_{f}) was determined using the equation F_{f}=1−Fᵤ where Fᵤ is the fraction unfolded. Fᵤ was determined experimentally using the following: Fᵤ=(Fₒ−F)/(U_{F}−F), where Fo is the ellipticity at 222 nm at a given temperature, F is the ellipticity where the peptides are folded, and U_{F} is the ellipticity where the peptides are unfolded.

CD studies were conducted to determine the relative heterodimeric affinities of the Vel, Coil 10, and Coil 148 light chain peptides (FIG. 42). Coil 10 (Tₘ=28.6° C.) has significantly decreased heterodimeric affinity compared to Vel (Tₘ=57.6° C.), as demonstrated by a lower melting temperature. The addition of V28L mutations in Coil 148 provide higher heterodimeric affinity compared to Coil 10 (Tₘ=39.7° C.).

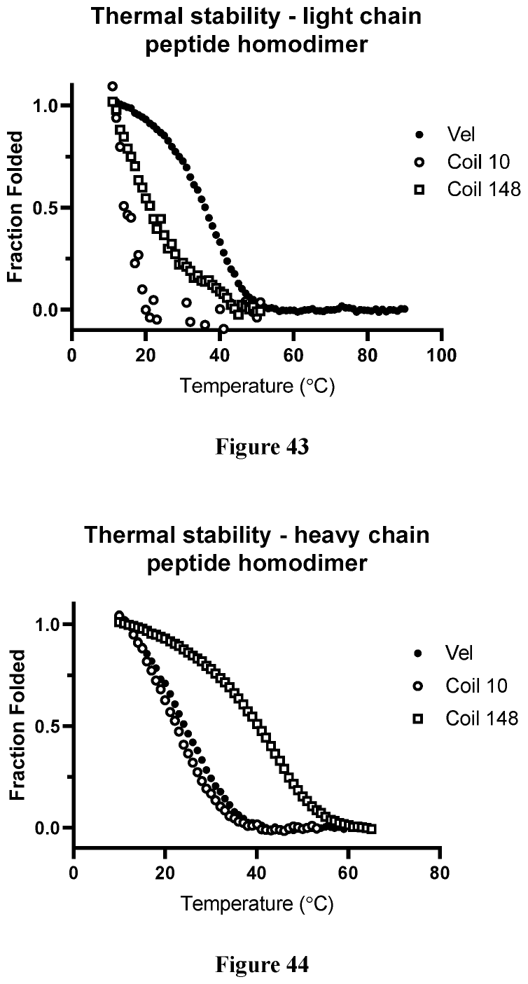

CD studies were also conducted to determine the relative homodimeric affinities of the Vel, Coil 10, and Coil 148 light chain peptides (FIG. 43). Both Coil 10 and Coil 148 (Tₘ=<20° C.) have significantly decreased light chain homodimer affinity compared to Vel (Tₘ=35.7° C.).

CD studies were conducted to determine the relative homodimeric affinities of the Vel, Coil 10, and Coil 148 heavy chain peptides (FIG. 44). Coil 148 (Tₘ=40.8° C.) has higher affinity than either the Vel (Tₘ=23.3° C.) or Coil 10 (Tₘ=21.0° C.) homodimer affinity.

Table of Certain Sequences

SEQ ID

| NQ | Description | Sequence |
|---|---|---|
| 1 | VelA | VAQLEEK VKTLRAE NYELKSE VQRLEEQ VAQL |
| 2 | VelB | VDELQAE VDQLEDE NYALKTK VAQLRKK VEKL |
| 3 | VelA with amino-terminal | QGASTTVAQLEEKVKTLRAENYELKSEVQRLEEQVAQL |
| | QGASTT | |
| 4 | VelB with amino-terminal | QGASTSVDELQAEVDQLEDENYALKTKVAQLRKKVEKL |
| | QGASTS | |
| 5 | VelA V7N | NAQLEEK VKTLRAE NYELKSE VQRLEEQ VAQL |
| 6 | VelA V14I | VAQLEEK IKTLRAE NYELKSE VQRLEEQ VAQL |
| 7 | VelA V14A | VAQLEEK AKTLRAE NYELKSE VQRLEEQ VAQL |
| 8 | VelA L17A | VAQLEEK VKTARAE NYELKSE VQRLEEQ VAQL |
| 9 | VelA Y22C | VAQLEEK VKTLRAE NCELKSE VQRLEEQ VAQL |
| 10 | VelA Y22S | VAQLEEK VKTLRAE NSELKSE VQRLEEQ VAQL |
| 11 | VelA L24A | VAQLEEK VKTLRAE NYEAKSE VQRLEEQ VAQL |
| 12 | VelA V28I | VAQLEEK VKTLRAE NYELKSE IQRLEEQ VAQL |
| 13 | VelA V28A | VAQLEEK VKTLRAE NYELKSE AQRLEEQ VAQL |
| 14 | VelA V28N | VAQLEEK VKTLRAE NYELKSE NQRLEEQ VAQL |
| 15 | VelA L31A | VAQLEEK VKTLRAE NYELKSE VQRAEEQ VAQL |
| 16 | VelA V35I | VAQLEEK VKTLRAE NYELKSE VQRLEEQ IAQL |
| 17 | VelA V35A | VAQLEEK VKTLRAE NYELKSE VQRLEEQ AAQL |
| 18 | VelA V35N | VAQLEEK VKTLRAE NYELKSE VQRLEEQ NAQL |
| 19 | VelA E11Q | VAQLQEK VKTLRAE NYELKSE VQRLEEQ VAQL |
| 20 | VelA E1IN | VAQLNEK VKTLRAE NYELKSE VQRLEEQ VAQL |
| 21 | VelA E11K | VAQLKEK VKTLRAE NYELKSE VQRLEEQ VAQL |
| 22 | VelA E12Q | VAQLEQK VKTLRAE NYELKSE VQRLEEQ VAQL |
| 23 | VelA E12N | VAQLENK VKTLRAE NYELKSE VQRLEEQ VAQL |
| 24 | VelA E12K | VAQLEKK VKTLRAE NYELKSE VQRLEEQ VAQL |
| 25 | VelA T16A | VAQLEEK VKALRAE NYELKSE VQRLEEQ VAQL |
| 26 | VelA E20Q | VAQLEEK VKTLRAQ NYELKSE VQRLEEQ VAQL |
| 27 | VelA E20N | VAQLEEK VKTLRAN NYELKSE VQRLEEQ VAQL |
| 28 | VelA E20K | VAQLEEK VKTLRAK NYELKSE VQRLEEQ VAQL |
| 29 | VelA E23Q | VAQLEEK VKTLRAE NYQLKSE VQRLEEQ VAQL |
| 30 | VelA E32Q | VAQLEEK VKTLRAE NYELKSE VQRLQEQ VAQL |
| 31 | VelA E32N | VAQLEEK VKTLRAE NYELKSE VQRLNEQ VAQL |
| 32 | VelA E32K | VAQLEEK VKTLRAE NYELKSE VQRLKEQ VAQL |
| 33 | VelA E33Q | VAQLEEK VKTLRAE NYELKSE VQRLEQQ VAQL |
| 34 | VelA E33N | VAQLEEK VKTLRAE NYELKSE VQRLENQ VAQL |
| 35 | VelA E33K | VAQLEEK VKTLRAE NYELKSE VQRLEKQ VAQL |
| 36 | VelA A8H | VHQLEEK VKTLRAE NYELKSE VQRLEEQ VAQL |
| 37 | VelA A8K | VKQLEEK VKTLRAE NYELKSE VQRLEEQ VAQL |
| 38 | VelA Q9H | VAHLEEK VKTLRAE NYELKSE VQRLEEQ VAQL |
| 39 | VelA Q9K | VAKLEEK VKTLRAE NYELKSE VQRLEEQ VAQL |
| 40 | VelA E11H | VAQLHEK VKTLRAE NYELKSE VQRLEEQ VAQL |
| 41 | VelA E12H | VAQLEHK VKTLRAE NYELKSE VQRLEEQ VAQL |
| 42 | VelA K15H | VAQLEEK VHTLRAE NYELKSE VQRLEEQ VAQL |
| 43 | VelA K15D | VAQLEEK VDTLRAE NYELKSE VQRLEEQ VAQL |
| 44 | VelA A19H | VAQLEEK VKTLRHE NYELKSE VQRLEEQ VAQL |
| 45 | VelA K25H | VAQLEEK VKTLRAE NYELHSE VQRLEEQ VAQL |
| 46 | VelA E33H | VAQLEEK VKTLRAE NYELKSE VQRLEHQ VAQL |
| 47 | VelA A36H | VAQLEEK VKTLRAE NYELKSE VQRLEEQ VHQL |
| 48 | VelA K25R | VAQLEEK VKTLRAE NYELRSE VQRLEEQ VAQL |
| 49 | VelA L24A/L31A | VAQLEEK VKTLRAE NYEAKSE VQRAEEQ VAQL |
| 50 | VelA L17A/L31A | VAQLEEK VKTARAE NYELKSE VQRAEEQ VAQL |
| 51 | VelA L17A/L24A/L31A | VAQLEEK VKTARAE NYEAKSE VQRAEEQ VAQL |
| 52 | VelA L31A/V35A | VKTARAE VKTLRAE NYELKSE VQRAEEQ AAQL |
| 53 | VelA V28A/L31A | VAQLEEK VKTLRAE NYELKSE AQRAEEQ VAQL |
| 54 | VelA V28A/L31A/V35A | VAQLEEK VKTLRAE NYELKSE AQRAEEQ AAQL |
| 55 | VelA L24A/V28A/L31A | VAQLEEK VKTLRAE NYEAKSE AQRAEEQ VAQL |
| 56 | VelA L31A/E33K | VAQLEEK VKTLRAE NYELKSE VQRAEKQ VAQL |
| 57 | VelA L24I | VAQLEEK VKTLRAE NYEIKSE VQRLEEQ VAQL |
| 58 | VelA L31I | VAQLEEK VKTLRAE NYELKSE VQRIEEQ VAQL |
| 59 | VelB V7N | NDELQAE VDQLEDE NYALKTK VAQLRKK VEKL |
| 60 | VelB V14A | VDELQAE ADQLEDE NYALKTK VAQLRKK VEKL |
| 61 | VelB V14I | VDELQAE IDQLEDE NYALKTK VAQLRKK VEKL |
| 62 | VelB V14N | VDELQAE NDQLEDE NYALKTK VAQLRKK VEKL |
| 63 | VelB L17I | VDELQAE VDQIEDE NYALKTK VAQLRKK VEKL |
| 64 | VelB Y22C | VDELQAE VDQLEDE NCALKTK VAQLRKK VEKL |
| 65 | VelB Y22S | VDELQAE VDQLEDE NSALKTK VAQLRKK VEKL |
| 66 | VelB L241 | VDELQAE VDQLEDE NYAIKTK VAQLRKK VEKL |
| 67 | VelB V28A | VDELQAE VDQLEDE NYALKTK AAQLRKK VEKL |
| 68 | VelB V28I | VDELQAE VDQLEDE NYALKTK IAQLRKK VEKL |
| 69 | VelB V28N | VDELQAE VDQLEDE NYALKTK NAQLRKK VEKL |
| 70 | VelB L31I | VDELQAE VDQLEDE NYALKTK VAQIRKK VEKL |
| 71 | VelB V35A | VDELQAE VDQLEDE NYALKTK VAQLRKK AEKL |
| 72 | VelB V35I | VDELQAE VDQLEDE NYALKTK VAQLRKK IEKL |
| 73 | VelB V35N | VDELQAE VDQLEDE NYALKTK VAQLRKK NEKL |
| 74 | VelB D8Q | VQELQAE VDQLEDE NYALKTK VAQLRKK VEKL |
| 75 | VelB D8N | VNELQAE VDQLEDE NYALKTK VAQLRKK VEKL |
| 76 | VelB E9N | VDNLQAE VDQLEDE NYALKTK VAQLRKK VEKL |
| 77 | VelB E9Q | VDQLQAE VDQLEDE NYALKTK VAQLRKK VEKL |
| 78 | VelB E13R | VDELQAR VDQLEDE NYALKTK VAQLRKK VEKL |
| 79 | VelB E13N | VDELQAN VDQLEDE NYALKTK VAQLRKK VEKL |
| 80 | VelB E13Q | VDELQAQ VDQLEDE NYALKTK VAQLRKK VEKL |
| 81 | VelB D15E | VDELQAE VEQLEDE NYALKTK VAQLRKK VEKL |
| 82 | VelB D15S | VDELQAE VSQLEDE NYALKTK VAQLRKK VEKL |
| 83 | VelB D15N | VDELQAE VNQLEDE NYALKTK VAQLRKK VEKL |
| 84 | VelB D19S | VDELQAE VDQLESE NYALKTK VAQLRKK VEKL |
| 85 | VelB D19N | VDELQAE VDQLENE NYALKTK VAQLRKK VEKL |
| 86 | VelB E20K | VDELQAE VDQLEDK NYALKTK VAQLRKK VEKL |
| 87 | VelB E20N | VDELQAE VDQLEDN NYALKTK VAQLRKK VEKL |
| 88 | VelB E20Q | VDELQAE VDQLEDQ NYALKTK VAQLRKK VEKL |
| 89 | VelB R32K | VDELQAE VDQLEDE NYALKTK VAQLKKK VEKL |
| 90 | VelB K33Q | VDELQAE VDQLEDE NYALKTK VAQLRQK VEKL |
| 91 | VelB K33E | VDELQAE VDQLEDE NYALKTK VAQLREK VEKL |
| 92 | VelB K37Q | VDELQAE VDQLEDE NYALKTK VAQLRKK VEQL |
| 93 | VelB K37E | VDELQAE VDQLEDE NYALKTK VAQLRKK VEEL |
| 94 | VelB D8K | VKELQAE VDQLEDE NYALKTK VAQLRKK VEKL |
| 95 | VelB D8H | VHELQAE VDQLEDE NYALKTK VAQLRKK VEKL |
| 96 | VelB E9K | VDKLQAE VDQLEDE NYALKTK VAQLRKK VEKL |
| 97 | VelB E9H | VDHLQAE VDQLEDE NYALKTK VAQLRKK VEKL |
| 98 | VelB Q11K | VDELKAE VDQLEDE NYALKTK VAQLRKK VEKL |
| 99 | VelB Q11H | VDELHAE VDQLEDE NYALKTK VAQLRKK VEKL |
| 100 | VelB A12K | VDELQKE VDQLEDE NYALKTK VAQLRKK VEKL |
| 101 | VelB A12H | VDELQHE VDQLEDE NYALKTK VAQLRKK VEKL |
| 102 | VelB D15K | VDELQAE VKQLEDE NYALKTK VAQLRKK VEKL |
| 103 | VelB D15H | VDELQAE VHQLEDE NYALKTK VAQLRKK VEKL |
| 104 | VelB D19K | VDELQAE VDQLEKE NYALKTK VAQLRKK |
| 105 | VelB D19H | VDELQAE VDQLEHE NYALKTK VAQLRKK VEKL |
| 106 | VelB K25H | VDELQAE VDQLEDE NYALHTK VAQLRKK VEKL |
| 107 | VelB K34H | VDELQAE VDQLEDE NYALKTK VAQLRKH VEKL |
| 108 | VelB E36H | VDELQAE VDQLEDE NYALKTK VAQLRKK VHKL |
| 109 | VelB K25R | VDELQAE VDQLEDE NYALRTK VAQLRKK VEKL |
| 110 | VelB L24I/L31I | VDELQAE VDQLEDE NYAIKTK VAQIRKK VEKL |
| 111 | VelB L17I/L31I | VDELQAE VDQIEDE NYALKTK VAQIRKK VEKL |
| 112 | VelB L17I/L24I/L31I | VDELQAE VDQIEDE NYAIKTK VAQIRKK VEKL |
| 113 | VelB L31I/V35I | VDELQAE VDQLEDE NYALKTK VAQIRKK IEKL |
| 114 | VelB V28I/L31I | VDELQAE VDQLEDE NYALKTK IAQIRKK VEKL |
| 115 | VelB V28I/L31I/V35I | VDELQAE VDQLEDE NYALKTK IAQIRKK IEKL |
| 116 | VelB D8K/E36H | VKELQAE VDQLEDE NYALKTK VAQLRKK VHKL |
| 117 | VelB D8K/E36H/L31I | VKELQAE VDQLEDE NYALKTK VAQIRKK VHKL |
| 118 | VelB D8K/D15K/L31I | VKELQAE VKQLEDE NYALKTK VAQIRKK VEKL |
| 119 | VelB D15K/E36H/L31I | VDELQAE VKQLEDE NYALKTK VAQIRKK VHKL |
| 120 | VelB L24A | VDELQAE VDQLEDE NYAAKTK VAQLRKK VEKL |
| 121 | VelB L31A | VDELQAE VDQLEDE NYALKTK VAQARKK VEKL |
| 141 | VelB L24I/V28I/L31I | VDELQAE VDQLEDE NYAIKTK IAQIRKK VEKL |
| 122 | IPV cleavage site | IPVSLRSG |
| 123 | M2 cleavage site | GPLGVR |
| 124 | MMP cleavage site 1 | LSGRSDNY |
| 125 | MMP cleavage site 2 | PLGVR |
| 126 | Linker 1 | GSGGS |
| 127 | Linker 2 | GGGS |
| 128 | Linker 3 | SGGGGGGGGGGS |
| 129 | Linker 4 | GGAA |
| 130 | Linker 5 | GGGGS |
| 131 | Linker 6 | LAAAA |
| 132 | Linker 7 | GGSG |
| 133 | Linker 8 | GGSGG |
| 134 | Linker 9 | GSGSG |
| 135 | Linker 10 | GSGGG |
| 136 | Linker 11 | GGGSG |
| 137 | Linker 12 | GSSSG |
| 138 | N-terminal sequence 1 | QGASTT |
| 139 | N-terminal sequence 2 | QGASTS |
| 140 | Linker-IPV cleavage site | GSIPVSLRSG |
| 142 | VelA L24Y | VAQLEEK VKTLRAE NYEYKSE VQRLEEQ VAQL |
| 143 | VelA L24W | VAQLEEK VKTLRAE NYEWKSE VQRLEEQ VAQL |
| 144 | VelA L24F | VAQLEEK VKTLRAE NYEFKSE VQRLEEQ VAQL |
| 145 | VelA L24S | VAQLEEK VKTLRAE NYESKSE VQRLEEQ VAQL |
| 146 | VelA L24G | VAQLEEK VKTLRAE NYEGKSE VQRLEEQ VAQL |
| 147 | VelA L24V | VAQLEEK VKTLRAE NYEVKSE VQRLEEQ VAQL |
| 148 | VelA L24I | VAQLEEK VKTLRAE NYEIKSE VQRLEEQ VAQL |
| 149 | VelA L24K | VAQLEEK VKTLRAE NYEKKSE VQRLEEQ VAQL |
| 150 | VelA L24V | VAQLEEK VKTLRAE NYEVKSE VQRLEEQ VAQL |
| 151 | VelA V28I | VAQLEEK VKTLRAE NYELKSE IQRLEEQ VAQL |
| 152 | VelA V28L | VAQLEEK VKTLRAE NYELKSE LQRLEEQ VAQL |
| 153 | VelA V28K | VAQLEEK VKTLRAE NYELKSE KQRLEEQ VAQL |
| 154 | VelA L24A/V28I | VAQLEEK VKTLRAE NYEAKSE IQRLEEQ VAQL |
| 155 | VelA L24A/V28L | VAQLEEK VKTLRAE NYEAKSE LQRLEEQ VAQL |
| 156 | VelA L24R/L31V | VAQLEEK VKTLRAE NYERKSE VQRVEEQ VAQL |
| 157 | VelA V28A/L31E | VAQLEEK VKTLRAE NYELKSE AQREEEQ VAQL |
| 158 | VelA L24R | VAQLEEK VKTLRAE NYERKSE VQRLEEQ VAQL |
| 159 | VelA L24K/L31V | VAQLEEK VKTLRAE NYEKKSE VQRVEEQ VAQL |
| 160 | VelA V28S | VAQLEEK VKTLRAE NYELKSE SQRLEEQ VAQL |
| 161 | VelA L31T | VAQLEEK VKTLRAE NYELKSE VQRTEEQ VAQL |
| 162 | VelA with amino-terminal | QGASAT VAQLEEK VKTLRAE NYELKSE VQRLEEQ VAQL |
| | QGASTT T5A | |
| 163 | VelA with amino-terminal | QGAS VAQLEEK VKTLRAE NYELKSE VQRLEEQ VAQL |
| | QGASTT ΔT5/ΔT6 | |
| 164 | VelA with amino-terminal | QGASTA VAQLEEK VKTLRAE NYELKSE VQRLEEQ VAQL |
| | QGASTT T6A | |
| 165 | VelA with amino-terminal | QGAATT VAQLEEK VKTLRAE NYELKSE VQRLEEQ VAQL |
| | QGASTT S4A | |
| 166 | VelA with amino-terminal | QGASTS VAQLEEK VKTLRAE NYELKSE VQRLEEQ VAQL |
| | QGASTT T6S | |
| 167 | VelA with amino-terminal | QGASTS VAQLEEK VKTLRAE NYEAKSE IQRLEEQ VAQL |
| | QGASTT T6S/L24A | |
| 168 | VelA with amino-terminal | QGASTS VAQLEEK VKTLRAE NYEAKSE LQRLEEQ VAQL |
| | QGASTT T6S/L24A/V28L | |
| 169 | VelB L24G | VDELQAE VDQLEDE NYAGKTK VAQLRKK VEKL |
| 170 | VelB L24A | VDELQAE VDQLEDE NYAAKTK VAQLRKK VEKL |
| 171 | VelB L24Y | VDELQAE VDQLEDE NYAYKTK VAQLRKK VEKL |
| 172 | VelB L24S | VDELQAE VDQLEDE NYASKTK VAQLRKK VEKL |
| 173 | VelB L24F | VDELQAE VDQLEDE NYAFKTK VAQLRKK VEKL |
| 174 | VelB L24W | VDELQAE VDQLEDE NYAWKTK VAQLRKK VEKL |
| 175 | VelB L24V | VDELQAE VDQLEDE NYAVKTK VAQLRKK VEKL |
| 176 | VelB L24K | VDELQAE VDQLEDE NYAKKTK VAQLRKK VEKL |
| 177 | VelB V28L | VDELQAE VDQLEDE NYALKTK LAQLRKK VEKL |
| 178 | VelB V28K | VDELQAE VDQLEDE NYALKTK KAQLRKK VEKL |
| 179 | VelB L24I/V28I | VDELQAE VDQLEDE NYAIKTK IAQLRKK VEKL |
| 180 | VelB L24V/V28I | VDELQAE VDQLEDE NYAVKTK IAQLRKK VEKL |
| 181 | VelB L24I/V28L | VDELQAE VDQLEDE NYAIKTK LAQLRKK VEKL |
| 182 | VelB L24S/L31Q | VDELQAE VDQLEDE NYASKTK VAQQRKK VEKL |
| 183 | VelB L24H | VDELQAE VDQLEDE NYAHKTK VAQLRKK VEKL |
| 184 | VelB V28S/L31W | VDELQAE VDQLEDE NYALKTK SAQWRKK VEKL |
| 185 | VelB L31P | VDELQAE VDQLEDE NYALKTK VAQPRKK VEKL |
| 186 | Vel light chain for CD | QGASTTVAQLEEKVKTLRAENYELKSEVQRLEEQVAQLGSIPVS |
| | studies | |
| 187 | Vel heavy chain for CD | QGASTSVDELQAEVDQLEDENYALKTKVAQLRKKVEKLGSIPVS |
| | studies | |
| 188 | Coil 10 light chain for CD | QGASTTVAQLEEKVKTLRAENYEAKSEVQRLEEQVAQLGSIPVS |
| | studies | |
| 189 | Coil 10 heavy chain for CD | QGASTSVDELQAEVDQLEDENYAIKTKVAQLRKKVEKLGSIPVS |
| | studies | |
| 190 | Coil 148 light chain for CD | QGASTTVAQLEEKVKTLRAENYEAKSELQRLEEQVAQLGSIPVS |
| | studies | |
| 191 | Coil 148 heavy chain for CD | QGASTSVDELQAEVDQLEDENYAIKTKLAQLRKKVEKLGSIPVS |
| | studies | |
| 192 | N-terminal residues of VelA | QGASTTVA |
| | with amino-terminal | |
| | QGASTT | |
| 193 | N-terminal modification 1 | QGASATVA |
| 194 | N-terminal modification 2 | QGASVA |
| 195 | N-terminal modification 3 | QGASTAVA |
| 196 | N-terminal modification 4 | QGAATTVA |
| 197 | N-terminal modification 5 | QGASTSVA |
| 198 | VelA masking domain with | QGASTTVAQLEEKVKTLRAENYELKSEVQRLEEQVAQL |
| | amino-terminal QGASTT | GSIPVSLRSG |
| | and linker IPV cleavage site | |
| 199 | VelB masking domain with | QGASTSVDELQAEVDQLEDENYALKTKVAQLRKKVEKL |
| | amino-terminal QGASTS | GSIPVSLRSG |
| | and linker IPV cleavage site | |

We claim:

1. A masked antibody comprising a first masking domain comprising a first coiled-coil domain and a second masking domain comprising a second coiled-coil domain, wherein the first masking domain or second masking domain is linked to a heavy chain of an antibody and the other of the first masking domain or second masking domain is linked to a light chain of the antibody, wherein the first coiled-coil domain comprises the sequence V₇D₈E₉L₁₀Q₁₁A₁₂E₁₃V₁₄D₁₅Q₁₆L₁₇E₁₈D₁₉E₂₀N₂₁Y₂₂A₂₃L₂₄K₂₅T₂₆K₂₇V₂₈A₂₉Q₃₀L₃₁R₃₂K₃₃K₃₄V₃₅ E₃₆K₃₇L₃₈ (SEQ ID NO: 2), and the second coiled-coil domain comprises the sequence V₇A₈Q₉L₁₀E₁₁E₁₂K₁₃V₁₄K₁₅T₁₆L₁₇R₁₈A₁₉E₂₀N₂₁Y₂₂E₂₃L₂₄K₂₅S₂₆E₂₇V₂₈Q₂₉R₃₀L₃₁E₃₂E₃₃Q₃₄V₃₅ A₃₆Q₃₇L₃₈ (SEQ ID NO: 1); wherein the first coiled-coil domain and/or the second coiled-coil domain comprises at least one amino acid substitution in SEQ ID NO: 2 or SEQ ID NO: 1 that reduces aggregation of the masked antibody in an aqueous formulation compared to the masked antibody without the at least one amino acid substitution in the same aqueous formulation.

2. The masked antibody of claim 1, wherein the at least one amino acid substitution reduces aggregation of the masked antibody in an aqueous formulation at pH6-8.5, pH 6.5, 6.6, 6.7, 6.8, 6.9, 7, 7.1, 7.2, 7.3, 7.4, 7.5, 7.6, 7.7, and/or 7.8.

3. The masked antibody of claim 1 or claim 2, wherein at least one amino acid substitution replaces an acidic amino acid with a non-acidic amino acid.

4. The masked antibody of claim 3, wherein at least one acidic amino acid is aspartic acid or glutamic acid.

5. The masked antibody of claim 3 or claim 4, wherein the non-acidic amino acid is any amino acid other than aspartic acid or glutamic acid.

6. The masked antibody of claim 5, wherein at least one acidic amino acid is replaced with a non-acidic amino acid selected from asparagine, glutamine, lysine, histidine, arginine, serine, phenylalanine, tyrosine, tryptophan, threonine, leucine, isoleucine, and methionine.

7. The masked antibody of claim 6, wherein at least one acidic amino acid is replaced with a non-acidic amino acid selected from asparagine, glutamine, lysine, histidine, arginine, and serine.

8. The masked antibody of any one of claims 3-7, wherein one, two, three, or four acidic amino acids in the first coiled-coil domain and/or one, two, three, or four acidic amino acids in the second coiled-coil domain are replaced with non-acidic amino acids.

9. The masked antibody of any one of claims 3-8, wherein at least one acidic amino acid selected from D8, E9, E13, D15, D19, and E36 in the first coiled-coil domain is replaced with a non-acidic amino acid.

10. The masked antibody of any one of claims 3-9, wherein at least one acidic amino acid selected from E11, E12, E20, E23, E32, and E33 in the second coiled-coil domain is replaced with a non-acidic amino acid.

11. The masked antibody of any one of claims 1-10, wherein the first coiled-coil domain comprises amino acid substitutions at D8 and E36.

12. The masked antibody of claim 11, wherein the first coiled-coil domain comprises amino acid substitutions D8K and E36H.

13. The masked antibody of any one of claims 1-12, wherein the first coiled-coil domain comprises a first substitution of a first hydrophobic amino acid with a less bulky hydrophobic amino acid or a more bulky hydrophobic amino acid, and the second coiled-coil domain comprises a second substitution of a second hydrophobic amino acid with a less bulky hydrophobic amino acid or a more bulky hydrophobic amino acid, wherein one coiled-coil domain comprises the less bulky hydrophobic amino acid substitution and one coiled-coil domain comprises the more bulky hydrophobic amino acid substitution, wherein the first hydrophobic amino acid and the second hydrophobic amino acid are at the same amino acid position in SEQ ID NOs: 2 and 1, respectively.

14. The masked antibody of claim 13, wherein the first hydrophobic amino acid and the second hydrophobic amino acid are both valine or leucine.

15. The masked antibody of claim 13 or claim 14, wherein the less bulky hydrophobic amino acid is alanine, glycine, or serine.

16. The masked antibody of any one of claims 13-15, wherein the more bulky hydrophobic amino acid is isoleucine, phenylalanine, tyrosine, tryptophan, or methionine.

17. The masked antibody of any one of claims 13-16, comprising wherein the first coiled-coil and the second coiled-coil comprise a substitution at position 24 and/or at position 28.

18. The masked antibody of any one of claims 13-17, wherein one, two, three, or four pairs of hydrophobic amino acids are substituted.

19. The masked antibody of any one of claims 13-18, wherein the first coiled-coil domain and the second coiled-coil domain comprise at least one pair of substitutions at at least one pair of positions selected from V14/V14, L17/L17, L24/L24, V28/V28, L31/L31, and V35/V35, wherein the first position is the position in the first coiled-coil domain and the second position is the position in the second coiled-coil domain.

20. The masked antibody of claim 19, wherein the first coiled-coil domain and the second coiled-coil domain comprise at least one pair of substitutions selected from V14A/V14I, V14I/V14A, L17A/L17I, L17I/L17A, L24A/L24I, L24I/L24A, L24G/L24Y, L24Y/L24G, L24A/L24Y, L24Y/L24A, L24Y/L24W, L24W/L24Y, L24Y/L24F, L24F/L24Y, L24S/L24F, L24F/L24S, L24Y/L24Y, L24S/L24S, L24G/L24W, L24W/L24G, L24G/L24F, L24F/L24G, L24A/L24W, L24W/L24A, L24W/L24F, L24F/L24W, L24A/L24F, L24F/L24A, L24W/L24S, L24S/L24W, L24Y/L24S, L24S/L24Y, L24F/L24F, L24I/L24V, L24V/L24I, L24K/L24I, L24I/L24K, V28A/V28I, V28I/V28A, V28N/V28N, V28L/V28I, V28I/V28L, V28K/V28I, V28I/V28K, V28L/V28L, L31A/L31I, L31I/L31A, V35A/V35I, V35I/V35A, and V35N/V35N.

21. The masked antibody of claim 20, wherein the first coiled-coil domain and the second coiled-coil domain comprise one pair of substitutions selected from: L24A/L24I, L24I/L24A, L24V/L24A, L24A/L24V, V28A/V28I, V28I/V28A, V28L/V28L, L31A/L31I, or L31I/L31A.

22. The masked antibody of claim 20, wherein the first coiled-coil domain and the second coiled-coil domain comprise two pairs of substitutions,
wherein the first pair of substitutions is selected from L24A/L24I and L24I/L24A, and the second pair of substitutions is selected from L31A/L311 and L311/L31A;
or wherein the first pair of substitutions is selected from L17A/L171 and L171/L17A, and the second pair of substitutions is selected from L31A/L311 and L31I/L31A;
or wherein the first pair of substitutions is selected from L17A/L171 and L17I/L17A, and the second pair of substitutions is selected from L24A/L24I and L24I/L24A;
or wherein the first pair of substitutions is selected from V28A/V281 and V28I/V28A, and the second pair of substitutions is selected from L31A/L311 and L31I/L31A;
or wherein the first pair of substitutions is selected from L24V/L24A and L24A/L24V, and the second pair of substitutions is V28L/V28L;
or wherein the first pair of substitutions is selected from L24I/L24A and L24A/L24I, and the second pair of substitutions is V28L/V28L.

23. The masked antibody of claim 20, wherein the first coiled-coil domain and the second coiled-coil domain comprise three pairs of substitutions, wherein the first pair of substitutions is selected from L17A/L17I and L17I/L17A, the second pair of substitutions is selected from L24A/L24I and L24I/L24A, and the third pair of substitutions is selected from L31A/L311 and L31I/L31A.

24. The masked antibody of any one of claims 1-23, wherein:
a) the first coiled-coil domain comprises substitutions L24A and L31A, and the second coiled-coil domain comprises substitutions L241 and L311; or
b) the first coiled-coil domain comprises substitutions L24I and L31I, and the second coiled-coil domain comprises substitutions L24A and L31A; or
c) the first coiled-coil domain comprises substitutions L17I and L31I, and the second coiled-coil domain comprises substitutions L17A and L31A; or
d) the first coiled-coil domain comprises substitutions L17I, L24I, and L31I, and the second coiled-coil domain comprises substitutions L17A, L24A, and L31A; or
e) the first coiled-coil domain comprises substitutions V281 and L31I, and the second coiled-coil domain comprises substitutions V28A and L31A; or
f) the first coiled-coil domain comprises substitution L31I, and the second coiled-coil domain comprises substitutions L31A and E33K; or
g) the first coiled-coil domain comprises substitutions D8K and E36H, and the second coiled-coil domain comprises no substitutions; or
h) the first coiled-coil domain comprises substitutions D8K, E36H, and L31I, and the second coiled-coil domain comprises substitution L31A; or
i) the first coiled-coil domain comprises substitutions D8K, D15K, and L31I, and the second coiled-coil domain comprises substitution L31A; or
j) the first coiled-coil domain comprises substitutions D15K, E36H, and L31I, and the second coiled-coil domain comprises substitution L31A.

25. The masked antibody of any one of claims 1-23, wherein:
a) the first coiled-coil domain comprises a substitution L24V, and the second coiled-coil domain comprises a substitution L24A; or
b) the first coiled-coil domain comprises a substitution L24A, and the second coiled-coil domain comprises a substitution L24V; or
c) the first coiled-coil domain comprises substitutions L24V and V28I, and the second coiled-coil domain comprises a substitution L24A; or
d) the first coiled-coil domain comprises a substitution L24A, and the second coiled-coil domain comprises substitutions L24V and V28I; or
e) the first coiled-coil domain comprises substitutions L241 and V28L, and the second coiled-coil domain comprises substitutions L24A and V28L; or
f) the first coiled-coil domain comprises substitutions L24A and V28L, and the second coiled-coil domain comprises substitutions L241 and V28L; or
g) the first coiled-coil domain comprises substitutions L24V and V281, and the second coiled-coil domain comprises substitutions T6S and L24A; or
h) the first coiled-coil domain comprises substitutions T6S and L24A, and the second coiled-coil domain comprises substitutions L24V and V28I; or
i) the first coiled-coil domain comprises substitutions L241 and V28L, and the second coiled-coil domain comprises substitutions T6S, L24A and V28L; or
j) the first coiled-coil domain comprises substitutions T6S, L24A and V28L, and the second coiled-coil domain comprises substitutions L241 and V28L.

26. The masked antibody of any one of claims 1-25, wherein the first coiled-coil domain comprises a sequence selected from SEQ ID NOs: 2, 59-121, 141, and 169-185.

27. The masked antibody of any one of claims 1-26, wherein the second coiled-coil domain comprises a sequence selected from SEQ ID NOs: 1, 5-58, 142-168.

28. The masked antibody of any one of claims 1-27, wherein the first coiled-coil domain and second coiled-coil domain comprise the sequences of SEQ ID NOs: 66 and 11, respectively; or SEQ ID NOs: 70 and 15, respectively; or SEQ ID NOs: 72 and 17, respectively; or SEQ ID NOs: 94 and 1, respectively; or SEQ ID NOs: 108 and 1, respectively; or SEQ ID NOs: 108 and 11, respectively; or SEQ ID NOs: 110 and 49, respectively; or SEQ ID NOs: 111 and 50, respectively; or SEQ ID NOs: 112 and 51, respectively; or SEQ ID NOs: 113 and 52, respectively; or SEQ ID NOs: 114 and 53, respectively; or SEQ ID NOs: 70 and 56, respectively; or SEQ ID NOs: 116 and 1, respectively; or SEQ ID NOs: 117 and 15, respectively; or SEQ ID NOs: 118 and 15, respectively; or SEQ ID NOs: 119 and 15, respectively; or SEQ ID NOs: 120 and 57, respectively; or SEQ ID NOs: 121 and 57, respectively; or SEQ ID NOs: 63 and 8, respectively.

29. The masked antibody of any one of claims 1-27, wherein the first coiled-coil domain and second coiled-coil domain comprise the sequences of SEQ ID NOs: 181 and 168, respectively; or SEQ ID NOs: 180 and 167, respectively; or SEQ ID NOs: 181 and 155, respectively; or SEQ ID NOs: 180 and 11, respectively.

30. The masked antibody of any one of claims 1-29, wherein each masking domain comprises an amino-terminal sequence selected from SEQ ID NOs: 138 and 139.

31. The masked antibody of claim 30, wherein the first masking domain comprises the amino-terminal sequence of SEQ ID NO: 139 and the second masking domain comprises the amino-terminal sequence of SEQ ID NO: 138.

32. The masked antibody of any one of claims 1-30, wherein each masking domain comprises an amino-terminal sequence of SEQ ID NO: 139.

33. A masked antibody comprising a first masking domain comprising a first coiled-coil domain and a second masking domain comprising a second coiled-coil domain, wherein the first masking domain or second masking domain is linked to a heavy chain of an antibody and the other of the first masking domain or second masking domain is linked to a light chain of the antibody, wherein the first coiled-coil domain and/or the second coiled-coil domain comprises at least one amino acid substitution that reduces aggregation of the masked antibody in an aqueous formulation compared to the masked antibody without the at least one amino acid substitution in the same aqueous formulation.

34. The masked antibody of claim 33, wherein the at least one amino acid substitution reduces aggregation of the masked antibody in an aqueous formulation at pH6-8.5, pH 6.5, 6.6, 6.7, 6.8, 6.9, 7, 7.1, 7.2, 7.3, 7.4, 7.5, 7.6, 7.7, and/or 7.8.

35. The masked antibody of claim 33 or claim 34, wherein at least one amino acid substitution reduces homodimerization of the first masking domain and/or the second masking domain.

36. The masking domain of any one of claims 33-35, wherein at least one amino acid substitution increases heterodimerization of the first masking domain and the second masking domain.

37. The masked antibody of any one of claims 33-36, wherein at least one amino acid substitution replaces an acidic amino acid in the first coiled coil domain or the second coiled coil domain with a non-acidic amino acid.

38. The masked antibody of claim 37, wherein at least one acidic amino acid is aspartic acid or glutamic acid.

39. The masked antibody of claim 37 or claim 38, wherein the non-acidic amino acid is any amino acid other than aspartic acid or glutamic acid.

40. The masked antibody of claim 39, wherein at least one acidic amino acid is replaced with a non-acidic amino acid selected from asparagine, glutamine, lysine, histidine, arginine, serine, phenylalanine, tyrosine, tryptophan, threonine, leucine, isoleucine, and methionine.

41. The masked antibody of claim 40, wherein at least one acidic amino acid is replaced with a non-acidic amino acid selected from asparagine, glutamine, lysine, histidine, arginine, and serine.

42. The masked antibody of any one of claims 37-41, wherein one, two, three, or four acidic amino acids in the first coiled-coil domain and/or one, two, three, or four acidic amino acids in the second coiled-coil domain are replaced with non-acidic amino acids.

43. The masked antibody of any one of claims 33-42, wherein the first coiled-coil domain comprises a first substitution of a first hydrophobic amino acid with a less bulky hydrophobic amino acid or a more bulky hydrophobic amino acid, and the second coiled-coil domain comprises a second substitution of a second hydrophobic amino acid with a less bulky hydrophobic amino acid or a more bulky hydrophobic amino acid, wherein one coiled-coil domain comprises the less bulky hydrophobic amino acid substitution and one coiled-coil domain comprises the more bulky hydrophobic amino acid substitution, wherein the first hydrophobic amino acid and the second hydrophobic amino acid are at corresponding amino acid positions in the first coiled-coil domain and second coiled-coil domain, respectively.

44. The masked antibody of claim 43, wherein the first hydrophobic amino acid and the second hydrophobic amino acid are both valine or leucine.

45. The masked antibody of claim 43 or claim 44, wherein the less bulky hydrophobic amino acid is alanine, glycine, or serine.

46. The masked antibody of any one of claims 43-45, wherein the more bulky hydrophobic amino acid is isoleucine, phenylalanine, tyrosine, tryptophan, or methionine.

47. The masked antibody of any one of claims 43-46, wherein one, two, three, or four pairs of hydrophobic amino acids are substituted.

48. The masked antibody of any one of claims 1-47, wherein each masking domain comprises a protease-cleavable linker and is linked to the heavy chain or light chain via the protease-cleavable linker.

49. The masked antibody of claim 48, wherein the protease-cleavable linker comprises a matrix metalloprotease (MMP) cleavage site, a urokinase plasminogen activator cleavage site, a matriptase cleavage site, a legumain cleavage site, a Disintegrin and Metalloprotease (ADAM) cleavage site, or a caspase cleavage site.

50. The masked antibody of claim 49, wherein the protease-cleavable linker comprises a MMP cleavage site.

51. The masked antibody of claim 50, wherein the MMP cleavage site is selected from an MMP2 cleavage site, an MMP7 cleavage site, an MMP9 cleavage site and an MMP13 cleavage site.

52. The masked antibody of claim 50 or claim 51, wherein the MMP cleavage site comprises the sequence IPVSLRSG (SEQ ID NO: 122) or GPLGVR (SEQ ID NO: 123).

53. The masked antibody of any one of claims 50-52, wherein each masking domain comprises a protease-cleavable linker of SEQ ID NO: 140.

54. The masked antibody of any one of claim 1-53, wherein the first masking domain is linked to the amino-terminus of the heavy chain and the second masking domain is linked to the amino-terminus of the light chain.

55. The masked antibody of any one of claims 1-53, wherein the first masking domain is linked to the amino-terminus of the light chain and the second masking domain is linked to the amino-terminus of the heavy chain.

56. The masked antibody of any one of claims 1-55, wherein the antibody binds a therapeutic antigen.

57. The masked antibody of claim 56, wherein the antibody is useful for treating cancer, an autoimmune disorder, or an infection.

58. The masked antibody of any one of claims 1-56, wherein the antibody binds a tumor-associated antigen.

59. The masked antibody of claim 58, wherein the tumor-associated antigen is selected from CD47, CD3, CD19, CD20, CD22, CD30, CD33, CD34, CD40, CD44, CD52, CD70, CD79a, CD123, Her-2, EphA2, lymphocyte associated antigen 1, VEGF or VEGFR, CTLA-4, LIV-1, nectin-4, CD74, SLTRK-6, EGFR, CD73, PD-L1, CD163, CCR4, CD147, EpCam, Trop-2, CD25, C5aR, Ly6D, alpha v integrin, B7H3, B7H4, Her-3, folate receptor alpha, GD-2, CEACAM5, CEACAM6, c-MET, CD266, MUC1, CD10, MSLN, sialyl Tn, Lewis Y, CD63, CD81, CD98, CD166, tissue factor (CD142), CD55, CD59, CD46, CD164, TGF beta receptor 1 (TGFBR1), TGFBR2, TGFBR3, FasL, MerTk, Axl, Clec12A, CD352, FAP, CXCR3, and CD5.

60. The masked antibody of any one of claims 1-59, wherein the masked antibody has reduced binding affinity for the antibody's target antigen compared to the binding affinity of the antibody for its target antigen without the first masking domain and second masking domain.

61. The masked antibody of claim 60, wherein the binding affinity of the masked antibody is reduced at least about 100-fold compared to the binding affinity of the antibody without the first masking domain and second masking domain.

62. The masked antibody of claim 61, wherein the binding affinity of the masked antibody is reduced between about 200-fold and about 1500-fold compared to the binding affinity of the antibody without the first masking domain and second masking domain.

63. The masked antibody of any one of claims 1-62, wherein the aqueous formulation comprises 10-100 mM of a buffering agent and 10-100 mM salt.

64. The masked antibody of claim 63, wherein the buffering agent comprises potassium phosphate and/or sodium phosphate and the salt comprises NaCl and/or KCl.

65. The masked antibody of claim 64, wherein the aqueous formulation is PBS.

66. The masked antibody of any one of claims 1-65, wherein the aqueous formulation comprises 1-20 mg/mL, 1-15 mg/mL, 1-10 mg/mL, 1-5 mg/mL, or 1-3 mg/mL masked antibody.

67. An immunoconjugate comprising the masked antibody of any one of claims 1-66 and a cytotoxic agent.

68. The immunoconjugate of claim 67, wherein the cytotoxic agent is an antitubulin agent, a DNA minor groove binding agent, a DNA replication inhibitor, a DNA alkylator, a topoisomerase inhibitor, a NAMPT inhibitor, or a chemotherapy sensitizer.

69. The immunoconjugate of claim 67 or claim 68, wherein the cytotoxic agent is an anthracycline, an auristatin, a camptothecin, a duocarmycin, an etoposide, an enediyine antibiotic, a lexitropsin, a taxane, a maytansinoid, a pyrrolobenzodiazepine, a combretastatin, a cryptophysin, or a vinca alkaloid.

70. The immunoconjugate of any one of claims 67-69, wherein the cytotoxic agent is auristatin E, AFP, AEB, AEVB, MMAF, MMAE, paclitaxel, docetaxel, doxorubicin, morpholino-doxorubicin, cyanomorpholino-doxorubicin, melphalan, methotrexate, mitomycin C, a CC-1065 analogue, CBI, calicheamicin, maytansine, an analog of dolastatin 10, rhizoxin, or palytoxin, epothilone A, epothilone B, nocodazole, colchicine, colcimid, estramustine, cemadotin, discodermolide, eleutherobin, a tubulysin, a plocabulin, or maytansine.

71. The immunoconjugate of claim 70, wherein the cytotoxic agent is an auristatin.

72. The immunoconjugate of claim 71, wherein the cytotoxic agent is MMAE or MMAF.

73. A nucleic acid sequence encoding the masked antibody of any one of claims 1-66.

74. An expression vector comprising the nucleic acid of claim 73.

75. A host cell comprising the nucleic acid of claim 73 or the expression vector of claim 70.

76. A host cell that expresses the masked antibody of any one of claims 1-66.

77. A method of producing the masked antibody of any one of claims 1-66 comprising culturing the host cell of claim 75 or claim 76 under conditions suitable for expressing the masked antibody.

78. The method of claim 77, further comprising isolating the masked antibody.

79. A pharmaceutical composition comprising the masked antibody of any one of claims 1-66 or the immunoconjugate of any one of claims 67-72 and a pharmaceutically acceptable carrier.

80. The masked antibody of any one of claims 1-66, the immunoconjugate of any one of claims 67-72, or the pharmaceutical composition of claim 79 for use in therapy.

81. The masked antibody of any one of claims 1-66, the immunoconjugate of any one of claims 67-72, or the pharmaceutical composition of claim 79 for use in treating cancer, an autoimmune disease, or an infection.

82. Use of the masked antibody of any one of claims 1-66, the immunoconjugate of any one of claims 67-72, or the pharmaceutical composition of claim 79 for the preparation of a medicament for use in therapy.

83. Use of the masked antibody of any one of claims 1-66, the immunoconjugate of any one of claims 67-72, or the pharmaceutical composition of claim 79 for the preparation of a medicament for use in treating cancer, an autoimmune disease, or an infection.

84. A method comprising administering to a subject in need thereof the masked antibody of any one of claims 1-66, the immunoconjugate of any one of claims 67-72, or the pharmaceutical composition of claim 79.

85. A method of treating cancer, an autoimmune disease, or an infection comprising administering to a subject in need thereof the masked antibody of any one of claims 1-66, the immunoconjugate of any one of claims 67-72, or the pharmaceutical composition of claim 79.

86. The method of claim 85, which is a method of treating cancer.

87. The method of claim 86, wherein the cancer is a solid cancer, a soft tissue cancer, or a hematopoietic cancer.

88. The method of claim 86 or claim 87, wherein the cancer is selected from lung cancer, pancreatic cancer, breast cancer, liver cancer, ovarian cancer, testicular cancer, kidney cancer, bladder cancer, spinal cancer, brain cancer, cervical cancer, endometrial cancer, colorectal cancer, anal cancer, endometrial cancer, esophageal cancer, gallbladder cancer, gastrointestinal cancer, gastric cancer, sarcoma, head and neck cancer, melanoma, skin cancer, prostate cancer, pituitary cancer, stomach cancer, uterine cancer, vaginal cancer, thyroid cancer, a sarcoma, soft tissue sarcoma, osteosarcoma, a lymphoma, diffuse large B-cell lymphomas (DLBCL), follicular lymphoma, myelodysplastic syndrome (MDS), Hodgkin's disease, a malignant lymphoma, non-Hodgkin's lymphoma, Burkitt's lymphoma, multiple myeloma, Richter's Syndrome, a leukemia, acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia (T-PLL), large granular lymphocytic leukemia, adult T-cell leukemia, and acute monocytic leukemia (AMOL).
